SEQUENCE LISTING (138 sequences)

SEQ ID NO: 1 - DNA, 2204 bp, Synechococcus PCC7942
agactacaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaag

SEQ ID NO: 2 - protein, 709 aa, Synechococcus PCC7942
MAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 3 - DNA, 2163 bp, Synechocystis PCC6803
atgagctattcatcaaaatacattttactaattagtgtccatggtttaattcggggagaaaaccttgagttgggcagagatgccgacaccggcgggcaaaccaaatatgtgctggaactggcccgggccttggtaaaaaatccccaggtggccagggtggatttgctgacccgtttaattaaagatcccaaagtagatgcagattatgcccagcctagagaacttattggcgatcgggcccagattgttcgcattgagtgcggcccggaggaatatattgccaaggaaatgctctgggactatttggataattttgctgaccatgccctggactatctcaaagaacagcccgaactgcccgatgtcatccatagccattacgccgatgcgggttacgtgggcaccagactttctcaccaattgggtattcctttggtgcacaccggacattccctgggtcgtagtaagcgcacccgtctcctgctcagtgggattaaagccgacgaaattgaaagccgttacaatatggcccgccggattaacgcggaggaagaaaccctaggatcagcggcgagggtgattaccagtacccatcaggaaatcgcagaacagtacgcccaatacgactattaccagccagaccagatgttggttattccccccggcactgatttagaaaagttttatccccccaaagggaacgagtgggaaacgcccattgttcaagagttgcaacgatttctacggcatccccgtaagcctattatcctcgctttgtcccgaccggatccccgcaaaaatatccataaattaattgcagcctatggccagtccccgcagttacaggcccaggccaatttggtcattgtggcgggcaatcgggatgacatcacggatctagaccaggggccgagggaagtactgacggatttactgttgaccattgaccgttacgatctctacggcaaagtggcttaccccaaacagaatcaggcggaggatgtgtatgctttgtttcgcctcactgctttatcccagggagtatttatcaatccggctttgacggaaccctttggtttaactttgattgaagcggcggcctgtggtgtgcccattgtggccacggaggatgggggcccggtggatattatcaaaaattgtcagaatggctatctaattaatcccctcgatgaagtggatattgcggataaattgctcaaagtactaaacgacaaacaacaatggcaattcctttctgaaagtggtctagagggagttaagcgccattattcttggccttcccacgttgaaagttatttagaagccatcaacgctctgacccaacagacttcagtgctgaaacgtagtgatttaaagcggcggcggactttgtactataacggtgccctggttactagtttggaccaaaatttactgggggcattacaggggggattaccgggcgatcgccagacgttggacgaattactggaagtgctgtatcaacatcgaaaaaatgtcggcttttgcattgccactgggagaagattggattcggtgctgaaaattttgcgggagtatcgcattccccaaccggatatgttgatcaccagcatgggcacggaaatttattcttccccggatttgatccccgaccagagttggcgcaatcacattgattatttgtggaaccgtaacgccattgtgcgtattttgggggaattacccggtttagccctccaacccaaggaagaactgagcgcctataaaattagctatttctacgatgcggcgatcgcccctaacctagaagaaattcggcaactgttgcataaaggggaacaaaccgtaaataccatcatttcctttggtcaatttttggatattctgcccatccgagcttccaaaggctatgctgtgcgttggttgagccaacagtggaatattcccctggagcacgttttcaccgccggaggatcgggagccgacgaagatatgatgcggggtaacaccctttccgtcgtcgtggctaaccgtcaccatgaggaactttctaatctaggggagatcgaaccgatttatttttccgaaaaacgttacgccgccggtattctggacggtctggcccattaccgcttctttgagttgttagaccccgtttaa

SEQ ID NO: 4 - protein, 720 aa, Synechocystis PCC6803
MSYSSKYILLISVHGLIRGENLELGRDADTGGQTKYVLELARALVKNPQVARVDLLTRLIKDPKVDADYAQPRELIGDRAQIVRIECGPEEYIAKEMLWDYLDNFADHALDYLKEQPELPDVIHSHYADAGYVGTRLSHQLGIPLVHTGHSLGRSKRTRLLLSGIKADEIESRYNMARRINAEEETLGSAARVITSTHQEIAEQYAQYDYYQPDQMLVIPPGTDLEKFYPPKGNEWETPIVQELQRFLRHPRKPIILALSRPDPRKNIHKLIAAYGQSPQLQAQANLVIVAGNRDDITDLDQGPREVLTDLLLTIDRYDLYGKVAYPKQNQAEDVYALFRLTALSQGVFINPALTEPFGLTLIEAAACGVPIVATEDGGPVDIIKNCQNGYLINPLDEVDIADKLLKVLNDKQQWQFLSESGLEGVKRHYSWPSHVESYLEAINALTQQTSVLKRSDLKRRRTLYYNGALVTSLDQNLLGALQGGLPGDRQTLDELLEVLYQHRKNVGFCIATGRRLDSVLKILREYRIPQPDMLITSMGTEIYSSPDLIPDQSWRNHIDYLWNRNAIVRILGELPGLALQPKEELSAYKISYFYDAAIAPNLEEIRQLLHKGEQTVNTIISFGQFLDILPIRASKGYAVRWLSQQWNIPLEHVFTAGGSGADEDMMRGNTLSVVVANRHHEELSNLGEIEPIYFSEKRYAAGILDGLAHYRFFELLDPV

SEQ ID NO: 5 - DNA, 735 bp, Synechocystis PCC6803
atgcgacagttattgctaatttctgacctggacaatacctgggtcggagatcaacaagccctggaacatttgcaagaatatctaggcgatcgccggggaaatttttatttggcctatgccacggggcgttcctaccattccgcgagggagttgcaaaaacaggtgggactcatggaaccggactattggctcaccgcggtggggagtgaaatttaccatccagaaggcctggaccaacattgggctgattacctctctgagcattggcaacgggatatcctccaggcgatcgccgatggttttgaggccttaaaaccccaatctcccttggaacaaaacccatggaaaattagctatcatctcgatccccaggcttgccccaccgtcatcgaccaattaacggagatgttgaaggaaaccggcatcccggtgcaggtgattttcagcagtggcaaagatgtggatttattgccccaacggagtaacaaaggtaacgccacccaatatctgcaacaacatttagccatggagccgtctcaaaccctggtgtgtggggactccggcaatgatattggcttatttgaaacttccgctcggggtgtcattgtccgtaatgcccagccggaattattgcactggtatgaccaatggggggattctcgtcattatcgggcccaatcgagccatgctggcgctatcctagaggcgatcgcccatttcgattttttgagctga

SEQ ID NO: 6 - protein, 244 aa, Synechocystis PCC6803
MRQLLLISDLDNTWVGDQQALEHLQEYLGDRRGNFYLAYATGRSYHSARELQKQVGLMEPDYWLTAVGSEIYHPEGLDQHWADYLSEHWQRDILQAIADGFEALKPQSPLEQNPWKISYHLDPQACPTVIDQLTEMLKETGIPVQVIFSSGKDVDLLPQRSNKGNATQYLQQHLAMEPSQTLVCGDSGNDIGLFETSARGVIVRNAQPELLHWYDQWGDSRHYRAQSSHAGAILEAIAHFDFLS

SEQ ID NO: 7 - DNA, 28 bp, Artificial
  primer for amplificaiton of asf
agactacaattggggcgttttctgtgag

SEQ ID NO: 8 - DNA, 49 bp, Artificial
  primer for amplificaiton of asf
cttacgtgccgatcaacgtctcattctgaaaaggttaagcgatcgcctc

SEQ ID NO: 9 - DNA, 39 bp, Artificial
  Primer for amplifying cat gene from pBeloBAC11
ttatcgcgatcgtcaggagctaaggaagctaaaatggag

SEQ ID NO: 10 - DNA, 29 bp, Artificial
  primer for amplificaiton of cat
cgaccaattcacgtgtttgacagcttatc

SEQ ID NO: 11 - DNA, 811 bp, Artificial
  cat gene amplified from pBeloBAC11
ttatcgcgatcgtcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacgtgaattggtcg

SEQ ID NO: 12 - DNA, 35 bp, Artificial
  primer for amplifying cat gene bearing the promoter from pBeloBAC11
ttttggcgatcgtgagacgttgatcggcacgtaag

SEQ ID NO: 13 - DNA, 29 bp, Artificial
  primer for amplifying cat gene bearing the promoter from pBeloBAC11
cgaccaattcacgtgtttgacagcttatc

SEQ ID NO: 14 - DNA, 901 bp, Artificial
  cat gene bearing the promoter amplified from pBeloBAC11
ttttggcgatcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacgtgaattggtcg

SEQ ID NO: 15 - DNA, 30 bp, Artificial
  primer for sequence verification of cat/asf
gcttctgcgttctgatttaatctgtatcag

SEQ ID NO: 16 - DNA, 27 bp, Artificial
  primer for sequence verification of cat/asf
tatcacttattcaggcgtagcaaccag

SEQ ID NO: 17 - DNA, 27 bp, Artificial
  primer for sequence verification of cat/asf
gtcgttagtgacatcgacaacacactg

SEQ ID NO: 18 - DNA, 26 bp, Artificial
  primer for sequence verification of cat/asf
gatcgcgatactgatcgagataggtc

SEQ ID NO: 19 - DNA, 10577 bp, Artificial
  Plasmid pLybAL11 containing ASF gene from Synechococcus elongatus PCC 7942
tgcatgcctgcaggtcgactctagaggatccccgggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 20 - DNA, 10667 bp, Artificial
  Plasmid pLybAL12 containing asf gene from Synechococcus elongatusn PCC 7942
tgcatgcctgcaggtcgactctagaggatccccgggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 21 - DNA, 28 bp, Artificial
  primer for amplifying Synechocystis spp. PCC 6803 pyrR (SphI/KpnI)
cggtgtgcatgccgttattgatggaatg

SEQ ID NO: 22 - DNA, 32 bp, Artificial
  primer for amplifying Synechocystis spp. PCC 6803 pyrR (SphI/KpnI)
tcactaggtacctaaattacctgggaagccag

SEQ ID NO: 23 - DNA, 474 bp, Synechocystis PCC6803
cggtgtgcatgccgttattgatggaatgggaagaagcaatggtcacaataaactggaggttatgggtatgttttttagccctaatgctccaatcgccttgattgtatcgaatgatgcagtctctaaaattgtatccgtaaaagacctctgcaccgccgacgggtctggattatgggcaataatcacagtcgagccagactacccctggaggtaaactccggggctggagccataaagattaggaattcattaagaaatgtaacaatcgacgttctagatcataccacgcccccactgtccggcagggtgaacagaggagactttcccctgttacagtgtcagtgacaaaacaactttttggcatcggtgcaggtggtgagccatggcggcccagatcattgaaattctttccccggaggaaatccgacgtacccttacccgtctggcttcccaggtaatttaggtacctagtga

SEQ ID NO: 24 - DNA, 35 bp, Artificial
  primer for amplifying Synechocystis spp. PCC 6803 nirA (SphI/KpnI)
cccaaggcatgcaggaaaacaagctcagaatgctg

SEQ ID NO: 25 - DNA, 42 bp, Artificial
  primer for amplifying Synechocystis spp. PCC 6803 nirA (SphI/KpnI)
tttattggtaccaacgcttcaagccagataacagtagagatc

SEQ ID NO: 26 - DNA, 384 bp, Synechocystis PCC6803
cccaaggcatgcaggaaaacaagctcagaatgctgcggggagaagggcaactccccaccagccccaaatttttgctggcgataaatatttttcggtttaattgttcacaaagctttttgaatttgagtttatagaaatttattggctggtaatgcttttttgcccccctgcaggacttcattgatccttgcctataccatcaatatcattggtcaataatgatgatgattgactaaaacatgtttaacaaaatttaacgcatatgctaaatgcgtaaactgcatatgccttggctgagtgtaatttacgttacaaattttaacgaaacgggaaccctatattgatctctactgttatctggcttgaagcgttggtaccaataaa

SEQ ID NO: 27 - DNA, 26 bp, Artificial
  primer for amplifying Synechococcus elongatus PCC 7942 psbAII (SphI/KpnI)
atctttgcgttccgtgacggctactg

SEQ ID NO: 28 - DNA, 25 bp, Artificial
  primer for amplifying Synechococcus elongatus PCC 7942 psbAII (SphI/KpnI)
gcagatggtaccggtcagcagagtg

SEQ ID NO: 29 - DNA, 676 bp, Synechococcus PCC7942
atctttgcgttccgtgacggctactgccagcatgccgagcctgatgtgtgacacctaagatcactccagttctctttggaaactggctgatgagtgaagacaccatctttggcaagatcatccggcgcgagattccagcagacattgtttatgaagatgatctctgtctggcttttcgagatgtggcaccccaagcgccggttcacattctggtgattcccaagcaaccaattgccaaccttttggaagcgacagcagaacatcaagcgctgctgggtcatttgttgctgactgtaaaggcgatcgcggcccaagaaggactcaccgagggctaccgcaccgtgattaacacgggccctgcgggtgggcaaaccgtttaccacctgcatattcacttactgggcgggcgatcgctggcttggccgcccggctgagaaaagtctgaaagttctttacaaaactcaatctgcttgttagattttactcacgaggctattaagtctcgtaaatagttcaactaaggactcatcgcaaaatgacgactgcattgcagcggcgcgagagcgccagcctgtggcagcagttctgcgagtgggtaaccagcaccgacaaccgcctctatgtgggttggttcggcgtgctgatgatccccactctgctgaccggtaccatctgc

SEQ ID NO: 30 - DNA, 40 bp, Artificial
  primer for amplifying Synechococcus elongatus PCC 7942 nirA
cagccagcatgcataaatttctgttttgaccaaaccatcc

SEQ ID NO: 31 - DNA, 34 bp, Artificial
  primer for amplifying Synechococcus elongatus PCC 7942 nirA
gtggctggtaccatggattcatctgcctacaaag

SEQ ID NO: 32 - DNA, 579 bp, Synechococcus PCC7942
cagccagcatgcataaatttctgttttgaccaaaccatcccgacataactcggtcagggcttgcaaaacagcggggatgcgatcgtgctgccagagactgcaaaggtgagccaataaccactgcgtctgccagtcatcaggtatcgcttggcagcgctgcaacccagcttcgaggacgcgaacatcaactgttttggccagttgctgaacctgtcgccaacaatgttcaaaatcaccgcttggccagccgtcactctctgcaaacgctgcatcagtcatgtgcaatcaatacaggttaaaaaccatgctaatggctccacctaagcgggcttcagagtcaaggcttgtagcaattgctactaaaaactgcgatcgctgctgaaatgagctggaattctgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcaagctgctttgtaggcagatgaatccatggtaccagccac

SEQ ID NO: 33 - DNA, 35 bp, Artificial
  primer for amplifying gammaPR (XbaI/KpnI)
gtgcattctagatggctacgagggcagacagtaag

SEQ ID NO: 34 - DNA, 50 bp, Artificial
  primer for amplifying gammaPR (XbaI/KpnI)
ttctgtggtaccatatggatcctccttcttaagatgcaaccattatcacc

SEQ ID NO: 35 - DNA, 1186 bp, Artificial
  gammaPR (XbaI/KpnI) promoter
gtgcattctagatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaattgttatcagctatgcgccgaccagaacaccttgccgatcagccaaacgtctcttcaggccactgactagcgataactttccccacaacggaacaactctcactgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggctttcttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatcttaagaaggaggatccatatggtaccacagaa

SEQ ID NO: 36 - DNA, 31 bp, Artificial
  primer for amplifying Synechocystis spp. PCC 6803 dnaK (SphI/KpnI)
gccccagcatgcaccagtaaacataaatctc

SEQ ID NO: 37 - DNA, 30 bp, Artificial
  primer for amplifying Synechocystis spp. PCC 6803 dnaK (SphI/KpnI)
attggtggtaccgaggtcaatcccaacaac

SEQ ID NO: 38 - DNA, 895 bp, Synechocystis PCC6803
gccccagcatgcaccagtaaacataaatctccccggcgacgcaaaaaacgggtgaccatcaagccggtgcgcttcggcatttttctgctttgcctagcaggcattgtggggggggcaactgccctaattatcaatcgtactggcgatcccctaggtgggttgctagaagaccccctagatgttttcctggaccaaccttcagaatttatccccgatgaagccacgagccggaatttgattctcagtcaacccaacttcaatcagcaagtgggtcagatggtagtacaaggctggcttgatagtaaaaagttagcctttggccaaaactacgatgtcggggcattgcagagtgttttagcccccaatctccttgcccaacaacggggtcgggcccaacgggatcaagcccaaaaggtctatcaccaatacgaacacaagttgcagattttagcctatcaagttaacccccaagaccccaaccgagccaccgttactgcccgggtagaagaaattagccagccctttaccctaggtaatcaacagcagaagggctccgccaccaaagatgacttgactgtgcgctatcagctagtacgacaccaaggggtttggaaaattgaccaaatacaagtggtaaatggcccccgttagtgcgtggcgttaactccccttttgaccaatggcatacggctagatgcccccataggtacggaaacctgcacttccgagaactaagcccctaccgtcactataagagtgtgaacgtgtcggccccaggcaatggattggaaccatggcttttcggcccatcgttgtgtcttatattcttacttgttaacgggagttaattaaaattatgggaaaagttgttgggattgacctcggtaccaccaat

SEQ ID NO: 39 - DNA, 28 bp, Artificial
  primer for amplifying Synechocystis spp. PCC 6803 kiaA (SphI/KpnI)
gccagagcatgcaaagctcactaactgg

SEQ ID NO: 40 - DNA, 31 bp, Artificial
  primer for amplifying Synechocystis spp. PCC 6803 kiaA (SphI/KpnI)
ggaaaaggtacctgagtctatgggcaacgtg

SEQ ID NO: 41 - DNA, 603 bp, Synechocystis PCC6803
gccagagcatgcaaagctcactaactgggcgggattttccgggtccggttgctgacggtaatagtcgtctaaaagtttggccacatccaaaaggctgtcggcggggggatgctggccggcgaggggattaattctgcttgtcatatacaaaaattgtaaaaaatggagggcggcgatcaggggcttagacacccaaatcctagccaaaaagggttaactagccaagggctatccatgggcaaagagataaaagaaaaagtctccaaatccctggtcatagagaaaaaattgccaaagttaccccaggccatacacggcccagcgccaagatggggagcacaaattcaaactttgtaaacaggccggaagctatccggccaaggagcactcagattgtgttaacgttcaggggagttgcttaacacaattttccaattaatagtattaatattttcttaacttgcaccgtaccatggtgagaaagcctatctgagcccttatttgattaaccttcgactgattattgatcccctgtgcagtctcccctctccctctgtctttttgctcccgaacacgttgcccatagactcaggtaccttttcc

SEQ ID NO: 42 - DNA, 30 bp, Artificial; identical to SEQ ID NO: 15
  primer for sequence analysis of pLybAL

SEQ ID NO: 43 - DNA, 27 bp, Artificial
  primer for sequence analysis of pLybAL
atgggtctgaatgtgcagaatgtagag

SEQ ID NO: 44 - DNA, 11090 bp, Artificial
  plasmid pLybAL15
tgcatgccgttattgatggaatgggaagaagcaatggtcacaataaactggaggttatgggtatgttttttagccctaatgctccaatcgccttgattgtatcgaatgatgcagtctctaaaattgtatccgtaaaagacctctgcaccgccgacgggtctggattatgggcaataatcacagtcgagccagactacccctggaggtaaactccggggctggagccataaagattaggaattcattaagaaatgtaacaatcgacgttctagatcataccacgcccccactgtccggcagggtgaacagaggagactttcccctgttacagtgtcagtgacaaaacaactttttggcatcggtgcaggtggtgagccatggcggcccagatcattgaaattctttccccggaggaaatccgacgtacccttacccgtctggcttcccaggtaatttaggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 45 - DNA, 11000 bp, Artificial
  plasmid pLybAL16
tgcatgcaggaaaacaagctcagaatgctgcggggagaagggcaactccccaccagccccaaatttttgctggcgataaatatttttcggtttaattgttcacaaagctttttgaatttgagtttatagaaatttattggctggtaatgcttttttgcccccctgcaggacttcattgatccttgcctataccatcaatatcattggtcaataatgatgatgattgactaaaacatgtttaacaaaatttaacgcatatgctaaatgcgtaaactgcatatgccttggctgagtgtaatttacgttacaaattttaacgaaacgggaaccctatattgatctctactgttatctggcttgaagcgttggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 46 - DNA, 11269 bp, Artificial
  plasmid pLybAL17
tgcatgccgagcctgatgtgtgacacctaagatcactccagttctctttggaaactggctgatgagtgaagacaccatctttggcaagatcatccggcgcgagattccagcagacattgtttatgaagatgatctctgtctggcttttcgagatgtggcaccccaagcgccggttcacattctggtgattcccaagcaaccaattgccaaccttttggaagcgacagcagaacatcaagcgctgctgggtcatttgttgctgactgtaaaggcgatcgcggcccaagaaggactcaccgagggctaccgcaccgtgattaacacgggccctgcgggtgggcaaaccgtttaccacctgcatattcacttactgggcgggcgatcgctggcttggccgcccggctgagaaaagtctgaaagttctttacaaaactcaatctgcttgttagattttactcacgaggctattaagtctcgtaaatagttcaactaaggactcatcgcaaaatgacgactgcattgcagcggcgcgagagcgccagcctgtggcagcagttctgcgagtgggtaaccagcaccgacaaccgcctctatgtgggttggttcggcgtgctgatgatccccactctgctgaccggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 47 - DNA, 11195 bp, Artificial
  plasmid pLybAL18
tgcatgcataaatttctgttttgaccaaaccatcccgacataactcggtcagggcttgcaaaacagcggggatgcgatcgtgctgccagagactgcaaaggtgagccaataaccactgcgtctgccagtcatcaggtatcgcttggcagcgctgcaacccagcttcgaggacgcgaacatcaactgttttggccagttgctgaacctgtcgccaacaatgttcaaaatcaccgcttggccagccgtcactctctgcaaacgctgcatcagtcatgtgcaatcaatacaggttaaaaaccatgctaatggctccacctaagcgggcttcagagtcaaggcttgtagcaattgctactaaaaactgcgatcgctgctgaaatgagctggaattctgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcaagctgctttgtaggcagatgaatccatggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 48 - DNA, 11820 bp, Artificial
  plasmid pLybAL19
tgcatgcctgcaggtcgactctagatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaattgttatcagctatgcgccgaccagaacaccttgccgatcagccaaacgtctcttcaggccactgactagcgataactttccccacaacggaacaactctcactgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggctttcttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatcttaagaaggaggatccatatggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 49 - DNA, 11511 bp, Artificial
  plasmid pLybAL21
tgcatgcaccagtaaacataaatctccccggcgacgcaaaaaacgggtgaccatcaagccggtgcgcttcggcatttttctgctttgcctagcaggcattgtggggggggcaactgccctaattatcaatcgtactggcgatcccctaggtgggttgctagaagaccccctagatgttttcctggaccaaccttcagaatttatccccgatgaagccacgagccggaatttgattctcagtcaacccaacttcaatcagcaagtgggtcagatggtagtacaaggctggcttgatagtaaaaagttagcctttggccaaaactacgatgtcggggcattgcagagtgttttagcccccaatctccttgcccaacaacggggtcgggcccaacgggatcaagcccaaaaggtctatcaccaatacgaacacaagttgcagattttagcctatcaagttaacccccaagaccccaaccgagccaccgttactgcccgggtagaagaaattagccagccctttaccctaggtaatcaacagcagaagggctccgccaccaaagatgacttgactgtgcgctatcagctagtacgacaccaaggggtttggaaaattgaccaaatacaagtggtaaatggcccccgttagtgcgtggcgttaactccccttttgaccaatggcatacggctagatgcccccataggtacggaaacctgcacttccgagaactaagcccctaccgtcactataagagtgtgaacgtgtcggccccaggcaatggattggaaccatggcttttcggcccatcgttgtgtcttatattcttacttgttaacgggagttaattaaaattatgggaaaagttgttgggattgacctcggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 50 - DNA, 11219 bp, Artificial
  plasmid pLybAL22
tgcatgcaaagctcactaactgggcgggattttccgggtccggttgctgacggtaatagtcgtctaaaagtttggccacatccaaaaggctgtcggcggggggatgctggccggcgaggggattaattctgcttgtcatatacaaaaattgtaaaaaatggagggcggcgatcaggggcttagacacccaaatcctagccaaaaagggttaactagccaagggctatccatgggcaaagagataaaagaaaaagtctccaaatccctggtcatagagaaaaaattgccaaagttaccccaggccatacacggcccagcgccaagatggggagcacaaattcaaactttgtaaacaggccggaagctatccggccaaggagcactcagattgtgttaacgttcaggggagttgcttaacacaattttccaattaatagtattaatattttcttaacttgcaccgtaccatggtgagaaagcctatctgagcccttatttgattaaccttcgactgattattgatcccctgtgcagtctcccctctccctctgtctttttgctcccgaacacgttgcccatagactcaggtaccgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagct

SEQ ID NO: 51 - DNA, 4962 bp, Artificial
  plasmid pLybAL13f
cgaccaattcacgtgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgacgttctgaaaaggttaagcgatcgcctcaaaaaagcgatagtgttttaaggcttccagaatgccattagcatagtggccctcagcaaaatagacgcgctcgtagctgcgcagtggctccaattccggtgagtaattgccaactacaacgccgagattatggcccttgagcatttcctcatcgttaccagaatcgcctgccaccaaaatgttctcaagaggaatccgccagcggagtgagaggtggcgaatcgcatcccctttcgaggcagctagcggcagaatgtcaagaaactcctgatgggaatagattgacttcagccgcaggcgatggcggcgaagatgttgccgtacttctcgcagcacagtctcgtggcgatcgcggacaaagaagctgactttgaagggtgtttgatcctcttccggctgcagttcaagaaagggtagttgtgccattactgcccgaattcgctgaggattccagttgcgattgatatgcttttcccagctgatatccggttcagcatcggtgccatagtgaatctcgctgccgacggaagtcacccagaagtttggcgaaggaacgccccactctttcaagacttcttgggcagagtctaggcgacgccccgtggcaattccaaaggcaaaatgatcgcgatactgatcgagataggtcattaaattctcgagtccttgacgatcgcccaacagtgtgttgtcgatgtcactaacgacaaggcgtttggcatcaatcaagcgtttgcgactccgtacgaaactgacagcacgacgacgaggcaaagccaccgtttccatgcgctcaaacagggtattgacatgttgatcccagctgtaatgggcgggaactttttcaatgccattgcggtgatagcactgccaaagatcgcgatcgctcagcagggtggcgagtgcagtcgcgatattagcgggtcggctgacatcaactaaagtgccgaaatcacagtgtttgagaatttcctgggggccgccatcatgggttgccaccaccggcacgccgcagcttcctgcctccaaaattgtcaaaccaaaaggttcggtcagcgccggattgacgaataccccgccggaatgagccgctaggcgatagaactccggcacatcatcagcctgatgctgtttgggataggcgacgctgccgtagaggtcgtagcgatcgaccagatggaaaatctcttggaacacctgccgactgccgcgatccatctggttgatgtcttggcggctgcccagtactaagacaaggttggcttttttgcgcagccaaggatgttcgccaaaggctcgcaccagcgccggtacatttttgcgaggtgcggggcgacagaggcagagaatttgaggtttttctgggtcgcgcagaaagcggctcagttcctgttggagaacaacaccgcgatcgcccaagggctgaaacctgaagcgatcggtatcgacacccggtggaatgacaagcttgcgctctgggttgtagcgatcgtaaacgcggtattgctcctccacttcctgctgagtgctggcgacaatccagtcagcatgagtgagcgtcatctcctccgcatcaattcgctgttgaatattgaattgcgcttcaatttcctcaagcggccagtcttgctccaacagcttttttagcttgatccgccccagagaatgccctgtgaaaattagcggtacattcaaccagcgactcagcagtgatcccacttggccagcatcagcatagtgggcctgaatccaagtcggggtgcgcttttgctgagccagatattggagaattgcatccgcaaaggtgtagagatggggccaaagcagctctttacggaggtagcgtttggggccaaaaggcaaacggacaatccgacctttgggcgcaaagggttcgatcgcctgactgtaaccaacactgacgcgggggtcggtgatttggcgggtgatgatgtcgacttgttggacttgtggggatttagcttgggcttgagccagttctaagacgtacttggtctgcccgccggtgtcggcatctcgccccagttccaagttctgccctcgcagcagaccatgggtctgaatgtgcagaatgtagagattttgagctgccacgcgctagtcagcctcacagaaaacgccccaattgtagtctaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 52 - DNA, 4962 bp, Artificial
  plasmid pLybAL13r
agactacaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaacgtcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacgtgaattggtcgaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 53 - DNA, 5052 bp, Artificial
  plasmid pLybAL14f
cgaccaattcacgtgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcacgttctgaaaaggttaagcgatcgcctcaaaaaagcgatagtgttttaaggcttccagaatgccattagcatagtggccctcagcaaaatagacgcgctcgtagctgcgcagtggctccaattccggtgagtaattgccaactacaacgccgagattatggcccttgagcatttcctcatcgttaccagaatcgcctgccaccaaaatgttctcaagaggaatccgccagcggagtgagaggtggcgaatcgcatcccctttcgaggcagctagcggcagaatgtcaagaaactcctgatgggaatagattgacttcagccgcaggcgatggcggcgaagatgttgccgtacttctcgcagcacagtctcgtggcgatcgcggacaaagaagctgactttgaagggtgtttgatcctcttccggctgcagttcaagaaagggtagttgtgccattactgcccgaattcgctgaggattccagttgcgattgatatgcttttcccagctgatatccggttcagcatcggtgccatagtgaatctcgctgccgacggaagtcacccagaagtttggcgaaggaacgccccactctttcaagacttcttgggcagagtctaggcgacgccccgtggcaattccaaaggcaaaatgatcgcgatactgatcgagataggtcattaaattctcgagtccttgacgatcgcccaacagtgtgttgtcgatgtcactaacgacaaggcgtttggcatcaatcaagcgtttgcgactccgtacgaaactgacagcacgacgacgaggcaaagccaccgtttccatgcgctcaaacagggtattgacatgttgatcccagctgtaatgggcgggaactttttcaatgccattgcggtgatagcactgccaaagatcgcgatcgctcagcagggtggcgagtgcagtcgcgatattagcgggtcggctgacatcaactaaagtgccgaaatcacagtgtttgagaatttcctgggggccgccatcatgggttgccaccaccggcacgccgcagcttcctgcctccaaaattgtcaaaccaaaaggttcggtcagcgccggattgacgaataccccgccggaatgagccgctaggcgatagaactccggcacatcatcagcctgatgctgtttgggataggcgacgctgccgtagaggtcgtagcgatcgaccagatggaaaatctcttggaacacctgccgactgccgcgatccatctggttgatgtcttggcggctgcccagtactaagacaaggttggcttttttgcgcagccaaggatgttcgccaaaggctcgcaccagcgccggtacatttttgcgaggtgcggggcgacagaggcagagaatttgaggtttttctgggtcgcgcagaaagcggctcagttcctgttggagaacaacaccgcgatcgcccaagggctgaaacctgaagcgatcggtatcgacacccggtggaatgacaagcttgcgctctgggttgtagcgatcgtaaacgcggtattgctcctccacttcctgctgagtgctggcgacaatccagtcagcatgagtgagcgtcatctcctccgcatcaattcgctgttgaatattgaattgcgcttcaatttcctcaagcggccagtcttgctccaacagcttttttagcttgatccgccccagagaatgccctgtgaaaattagcggtacattcaaccagcgactcagcagtgatcccacttggccagcatcagcatagtgggcctgaatccaagtcggggtgcgcttttgctgagccagatattggagaattgcatccgcaaaggtgtagagatggggccaaagcagctctttacggaggtagcgtttggggccaaaaggcaaacggacaatccgacctttgggcgcaaagggttcgatcgcctgactgtaaccaacactgacgcgggggtcggtgatttggcgggtgatgatgtcgacttgttggacttgtggggatttagcttgggcttgagccagttctaagacgtacttggtctgcccgccggtgtcggcatctcgccccagttccaagttctgccctcgcagcagaccatgggtctgaatgtgcagaatgtagagattttgagctgccacgcgctagtcagcctcacagaaaacgccccaattgtagtctaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 54 - DNA, 5052 bp, Artificial
  plasmid pLybAL14r
agactacaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaacgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacgtgaattggtcgaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcattttagcttccttagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 55 - DNA, 24 bp, Artificial
  primer for detection of plasmid in cyanobacteria
ggtggttgtgtttgacagcttatc

SEQ ID NO: 56 - DNA, 651 bp, Synechocystis PCC6803
atggcttctcaattacgtgtttatgtgccggagcatcctctaattaagcattggttgggggtagctagggatgaaaacacgccgccggttttgtttaaaactgccatgggggaattgggacgttggttgacctatgaggccgctcgttattggttgccgacggtggatacggaagtgaaaactcccctggcgatcgccaaggccagtcttattgacccccaaacgccctttgtcattgtgcccattttgcgggcggggttggctctggtggaaggggcccaggggttgttgcccctggcaaaaatttaccatctgggtttagtgcgcaatgaaactaccctggaacctagtctgtatctgaacaagttgccggagcggtttgcccccggtacccatcttttgttgctagatcccatgttggctacgggtaataccatcatggctgctttggatttgctgatggcccgggacattgatgccaatttaatccgtttggtctccgtggtggccgcccccactgccctgcaaaaattaagtaatgcccatcccaatttgaccatctacaccgccatgattgacgaacaactcaatgaccggggttacattgtgcccggcctaggggatgcaggcgatcgttgctttggtacttga

SEQ ID NO: 57 - protein, 216 aa, Synechocystis PCC6803
MASQLRVYVPEHPLIKHWLGVARDENTPPVLFKTAMGELGRWLTYEAARYWLPTVDTEVKTPLAIAKASLIDPQTPFVIVPILRAGLALVEGAQGLLPLAKIYHLGLVRNETTLEPSLYLNKLPERFAPGTHLLLLDPMLATGNTIMAALDLLMARDIDANLIRLVSVVAAPTALQKLSNAHPNLTIYTAMIDEQLNDRGYIVPGLGDAGDRCFGT

SEQ ID NO: 58 - DNA, 654 bp, Synechococcus PCC7942
atggctcctcaactgcgtatcttcgtgccgccccatcccttaattcggcactggctgggcattgcccgcgatcgccagacgccgacgcctctgtttcgcaccgcgatcgcagagctgggccgctggctcgcctatgaggctgtgcgggaatggctaccaacgattccagcggcggtgcaaactcctcttgcagaaaccccagcggagttcgtcgatttttcgcaacccttggcgatcgtgccgattctgcgcgcaggtctgggtttagtggagtctgtccaacaggttttgccgactgcccgcatttttcacgtgggtctcaagcgggatgaagtcagtcttgaaccgcgctgctacctcaatcacctgccagagcaacttgaagtgaacagtcgcgttctggttctcgacccgatgctggcgacaggtggctcgctgctctatacccttgatttgctgcgcgatcgcggtgtctctgctgagcaagtgcgggtgctttcaattgtggctgccccgccagcgctacaaaaactcagtcaagcctacccggcgttgacgatttacagcgccatcattgatgagcagctgaacgacaaaggctttatcgtgccggggctgggggatgctggcgatcgcctgtttggtactccttga

SEQ ID NO: 59 - protein, 217 aa, Synechococcus PCC7942
MAPQLRIFVPPHPLIRHWLGIARDRQTPTPLFRTAIAELGRWLAYEAVREWLPTIPAAVQTPLAETPAEFVDFSQPLAIVPILRAGLGLVESVQQVLPTARIFHVGLKRDEVSLEPRCYLNHLPEQLEVNSRVLVLDPMLATGGSLLYTLDLLRDRGVSAEQVRVLSIVAAPPALQKLSQAYPALTIYSAIIDEQLNDKGFIVPGLGDAGDRLFGTP

SEQ ID NO: 60 - DNA, 33 bp, Artificial
  primer for amplifying upp gene from Bacillus subtilis 168
aagaagcaagacagcgtgtagctgctctgactg

SEQ ID NO: 61 - DNA, 50 bp, Artificial
  primer for amplifying upp gene from Bacillus subtilis 168
tcccgggatttggtaccttattttgttccaaacatgcggtcacccgcatc

SEQ ID NO: 62 - DNA, 849 bp, Bacillus subtilis
aagaagcaagacagcgtgtagctgctctgactgataaatttcctttatataaagaattagattattaagatcctaaaacccgcttgggcttatgcccggcgggttttttgacgatgttcttgaaactcaatgtctttttttgtagaatcaatagaagtgtgtaattgttgatgggacaataaaaaaggagctgaaacacagtatgggaaaggtttatgtatttgatcatcctttaattcagcacaagctgacatatatacggaatgaaaatacaggtacgaaggattttagagagttagtagatgaagtggctacactcatggcatttgaaattacccgcgatcttcctctggaagaagtggatatcaatacaccggttcaggctgcgaaatcgaaagtcatctcagggaaaaaactcggagtggttcctatcctcagagcaggattgggaatggttgacggcattttaaagctgattcctgcggcaaaagtgggacatgtcggcctttaccgtgatccagaaaccttaaaacccgtggaatactatgtcaagcttccttctgatgtggaagagcgtgaattcatcgtggttgacccgatgctcgctacaggcggttccgcagttgaagccattcacagccttaaaaaacgcggtgcgaaaaatatccgtttcatgtgtcttgtagcagcgccggagggtgtggaagaattgcagaagcatcattcggacgttgatatttacattgcggcgctagatgaaaaattaaatgaaaaaggatatattgttccaggtctcggagatgcgggtgaccgcatgtttggaacaaaataaggtaccaaatcccggga

SEQ ID NO: 63 - DNA, 22 bp, Artificial
  primer for sequence verification of pLybAL7f
gtaatacgactcactatagggc

SEQ ID NO: 64 - DNA, 24 bp, Artificial
  primer for sequence verification of pLybAL7f
cacacaggaaacagctatgaccat

SEQ ID NO: 65 - DNA, 8988 bp, Artificial
  plasmid pLybAL7f
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccactcccgggatttggtaccttattttgttccaaacatgcggtcacccgcatctccgagacctggaacaatatatcctttttcatttaatttttcatctagcgccgcaatgtaaatatcaacgtccgaatgatgcttctgcaattcttccacaccctccggcgctgctacaagacacatgaaacggatatttttcgcaccgcgttttttaaggctgtgaatggcttcaactgcggaaccgcctgtagcgagcatcgggtcaaccacgatgaattcacgctcttccacatcagaaggaagcttgacatagtattccacgggttttaaggtttctggatcacggtaaaggccgacatgtcccacttttgccgcaggaatcagctttaaaatgccgtcaaccattcccaatcctgctctgaggataggaaccactccgagttttttccctgagatgactttcgatttcgcagcctgaaccggtgtattgatatccacttcttccagaggaagatcgcgggtaatttcaaatgccatgagtgtagccacttcatctactaactctctaaaatccttcgtacctgtattttcattccgtatatatgtcagcttgtgctgaattaaaggatgatcaaatacataaacctttcccatactgtgtttcagctccttttttattgtcccatcaacaattacacacttctattgattctacaaaaaaagacattgagtttcaagaacatcgtcaaaaaacccgccgggcataagcccaagcgggttttaggatcttaataatctaattctttatataaaggaaatttatcagtcagagcagctacacgctgtcttgcttcttgtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 66 - DNA, 31 bp, Artificial
  primer for amplifying kanamycin resistance marker vector pLybAA1
gtcagtgcactgctctgccagtgttacaacc

SEQ ID NO: 67 - DNA, 38 bp, Artificial
  primer for amplifying kanamycin resistance marker vector pLybAA1
ctcagtggcgccaaaactcacgttaagggattttggtc

SEQ ID NO: 68 - DNA, 950 bp, Artificial
  kanamycin resistance marker from vector pLybAA1, originally derived from pACYC177
gtcagtgcactgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttggcgccactgag

SEQ ID NO: 69 - DNA, 9864 bp, Artificial
  plasmid pLybAL8f (kanamycin resistance marker plus pLybAL7f)
gcggccgcaaggggttcgcgtcagcgggtgttggcgggtgtcggggctggcttaactatgcggcatcagagcagattgtactgagagtgcactgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccactcccgggatttggtaccttattttgttccaaacatgcggtcacccgcatctccgagacctggaacaatatatcctttttcatttaatttttcatctagcgccgcaatgtaaatatcaacgtccgaatgatgcttctgcaattcttccacaccctccggcgctgctacaagacacatgaaacggatatttttcgcaccgcgttttttaaggctgtgaatggcttcaactgcggaaccgcctgtagcgagcatcgggtcaaccacgatgaattcacgctcttccacatcagaaggaagcttgacatagtattccacgggttttaaggtttctggatcacggtaaaggccgacatgtcccacttttgccgcaggaatcagctttaaaatgccgtcaaccattcccaatcctgctctgaggataggaaccactccgagttttttccctgagatgactttcgatttcgcagcctgaaccggtgtattgatatccacttcttccagaggaagatcgcgggtaatttcaaatgccatgagtgtagccacttcatctactaactctctaaaatccttcgtacctgtattttcattccgtatatatgtcagcttgtgctgaattaaaggatgatcaaatacataaacctttcccatactgtgtttcagctccttttttattgtcccatcaacaattacacacttctattgattctacaaaaaaagacattgagtttcaagaacatcgtcaaaaaacccgccgggcataagcccaagcgggttttaggatcttaataatctaattctttatataaaggaaatttatcagtcagagcagctacacgctgtcttgcttcttgtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 70 - DNA, 1470 bp, Synechocystis PCC6803
atgaaatccccccaggctcaacaaatcctagaccaggcccgccgtttgctctacgaaaaagccatggtcaaaatcaatgggcaatacgtggggacggtggcggccattccccaatcggatcaccatgatttgaactatacggaagttttcattcgggacaatgtgccggtgatgatcttcttgttactgcaaaatgaaacggaaattgtccaaaactttttggaaatttgcctcaccctccaaagtaagggctttcccacctacggcatttttcccactagttttgtggaaacggaaaaccatgaactcaaggcagactatggccaacgggcgatcggtcgagtttgctcggtggatgcgtccctctggtggcctattttggcctattactacgtgcaaagaaccggcaatgaagcctgggctagacaaacccatgtgcaattggggctacaaaagtttttaaacctcattctccatccagtctttcgggatgcacccactttgtttgtgcccgacggggcctttatgattgaccgccccatggatgtgtggggagcgccgttggaaatccaaaccctgctctacggagccctgaaaagtgcggcggggttactgttaatcgacctcaaggcgaagggttattgcagcaataaagaccatccttttgacagcttcacgatggagcagagtcatcaatttaacctgagtgtggattggctcaaaaaactccgcacctatctgctcaagcattattggattaattgcaatattgtccaagctctccgccgccgtcccacggaacagtacggtgaagaagccagcaacgaacataatgtccacacagaaaccattcccaactggctccaggattggctcggcgatcggggaggctatttaatcggcaatatccgcacgggtcgccccgattttcgctttttctccctgggtaattgcttgggggcaattttcgatgtcactagcttggcccagcaacgttcctttttccgtttggtattaaataatcagcgggagttatgtgcccaaatgcccctgaggatttgccatccccccctcaaagatgacgattggcgcagtaaaaccggctttgaccgcaaaaatttaccctggtgctaccacaacgccggccattggccctgtttattttggtttctggtggtggcggtgctccgccatagctgccattccaactacggcacggtggagtatgcggaaatggggaacctaattcgcaataactatgaggtgcttttgcgccgtttgcccaagcataaatgggctgaatattttgatggccccacgggcttttgggtcgggcaacaatcccgttcctaccaaacctggaccattgtgggcctattgctagtacaccatttcacagaagttaaccccgacgatgctttgatgttcgatttgcctagtttgaaaagtttgcatcaagcgctgcattaa

SEQ ID NO: 71 - protein, 489 aa, Synechocystis PCC6803
MKSPQAQQILDQARRLLYEKAMVKINGQYVGTVAAIPQSDHHDLNYTEVFIRDNVPVMIFLLLQNETEIVQNFLEICLTLQSKGFPTYGIFPTSFVETENHELKADYGQRAIGRVCSVDASLWWPILAYYYVQRTGNEAWARQTHVQLGLQKFLNLILHPVFRDAPTLFVPDGAFMIDRPMDVWGAPLEIQTLLYGALKSAAGLLLIDLKAKGYCSNKDHPFDSFTMEQSHQFNLSVDWLKKLRTYLLKHYWINCNIVQALRRRPTEQYGEEASNEHNVHTETIPNWLQDWLGDRGGYLIGNIRTGRPDFRFFSLGNCLGAIFDVTSLAQQRSFFRLVLNNQRELCAQMPLRICHPPLKDDDWRSKTGFDRKNLPWCYHNAGHWPCLFWFLVVAVLRHSCHSNYGTVEYAEMGNLIRNNYEVLLRRLPKHKWAEYFDGPTGFWVGQQSRSYQTWTIVGLLLVHHFTEVNPDDALMFDLPSLKSLHQALH

SEQ ID NO: 72 - DNA, 1392 bp, Synechococcus PCC7942
atgcccgattctgttgtgctgcccgctacgctgcagaccgcgctgcaaacagcggagcagttactttgggatcgggccttggttcgctatcacgatcagtgggcgggggcgatcgcggcactgcctgaagatcaggagttggcggcagcgaactaccgcgaaatctttattcgcgacaacgtgccggtgatgctctacctgctgttgcagggcaaaactgacgttgtccgcgacttcttgcaactgtcgctttctctccagagccaggcactgcaaacctatggcattctgccgaccagtttcgtctgtgaggaaacccactgcgttgctgactatggtcagcgggcgatcgggcgggtggtttctgctgaccctagcctttggtggccggtgctgctacaggcctatcggcgggcctcccatgatgatgcctttgtccacagtccgactgttcagcaggggttacagcggttgctggctttcctgctgcgtccggttttcaaccaaaacccactgctcgaggtgcccgatggggccttcatggtcgatcgtcccttggatgtggcgggcgcacctttagaaattcaagtcctgctctacggggcactgcgggcttgtgggcagttgctgcaatacaccgaagcggccaatgctgcccatgtgcaagcccgtcgcctgcggcagtatctctgctggcactactgggtgacgcccgatcgcctgcgacgctggcagcagtggcccaccgaagaatttggcgatcgcagccataacccctacaacattcagccgatcgccatccctgactgggttgaaccttggctgggtgagtcgggtggctacttcctagggaacatacgggcaggacgtcctgacttccgcttttttagccttggcaatttgctggcgatcgttttcgatgtgcttccgctcaatcagcagggtgcgattctgcgcttgattttgcagaacgaagcccagattttgggccaagtgccgttgcggctctgctatcccgctttaaccggatcggcgtggaaaatcctgacgggttgcgatcctaaaaatcagccttggtcctatcacaacggtggtagttggccatccctgctttggtatctcagtgcggcggtcttgcactaccaacagcggggaggcgatcgcaatctctgtcaggtctggctgaataagcttcagcactaccacactcagcagtgcgagcaactccctggcgatgagtggccagagtactacgagggtcaggactcggtccagattgctactcgcgcctgccgttatcagacttggacgtttacgggattgctgctgaatcacgcactgctctcgcagccccagggcattcaactgctgagtctgcggggcttaccctaa

SEQ ID NO: 73 - protein, 463 aa, Synechococcus PCC7942
MPDSVVLPATLQTALQTAEQLLWDRALVRYHDQWAGAIAALPEDQELAAANYREIFIRDNVPVMLYLLLQGKTDVVRDFLQLSLSLQSQALQTYGILPTSFVCEETHCVADYGQRAIGRVVSADPSLWWPVLLQAYRRASHDDAFVHSPTVQQGLQRLLAFLLRPVFNQNPLLEVPDGAFMVDRPLDVAGAPLEIQVLLYGALRACGQLLQYTEAANAAHVQARRLRQYLCWHYWVTPDRLRRWQQWPTEEFGDRSHNPYNIQPIAIPDWVEPWLGESGGYFLGNIRAGRPDFRFFSLGNLLAIVFDVLPLNQQGAILRLILQNEAQILGQVPLRLCYPALTGSAWKILTGCDPKNQPWSYHNGGSWPSLLWYLSAAVLHYQQRGGDRNLCQVWLNKLQHYHTQQCEQLPGDEWPEYYEGQDSVQIATRACRYQTWTFTGLLLNHALLSQPQGIQLLSLRGLP

SEQ ID NO: 74 - DNA, 966 bp, Synechocystis PCC6803
atgattaattgtcaattttgttccgttatttccaaatctaacggggaagatcctatcggcacagcaaattcaagtgatcgttggttaattatggaattaccccaaccttggacagaggaacgctttcatcatgaccccattcttaaaccaattcatgatctttttcatcaactttctgatcaaggagttaaagtatctccaatggcgatcgcctcagatcacgagtattctcaatcaggatttagtcgtattattcactaccaaaagtttaatttgctcttttccagttttataaaagaagaatatttagttcctgatgatcaaaggtgggatcttatcaaaaatttatgttatcaatctccagagttagaaaattttcgtaactataaactgtcagatgttgttgatcgagatatgatggtatgtactcatggaaacattgatgtggcttgttcgagatttggttatcctatttataaacaattacgacaaaaatatgcatcaaaaaatttaagaatatggcgctgctctcattttgggggacatcagtttgctccgactttaattgattttccaaatgggcaagtttggggacatcttgagtctgaagttttagataatctggtaaggcaagaaggtcaagttaaacaactttataaattttatcgaggttgggtaggcgtaacaaaatttgcccagattgttgagcgtgaaatttggactcaacgaggttggcaatggttaaattatcaaaaatcagctcaaatattgaacatggatgataatcagcatgatcccaattgggtagaggttcaatttgattttatttctcccgataaagttaaaggagcttattttgcaagagttgaagtcaatgggtcagtgatgactgctagaaattcaggagatgaacttatttctgtcaagcagtatagtgtcagctacttaaaagaaattgataaataa

SEQ ID NO: 75 - protein, 321 aa, Synechocystis PCC6803
MINCQFCSVISKSNGEDPIGTANSSDRWLIMELPQPWTEERFHHDPILKPIHDLFHQLSDQGVKVSPMAIASDHEYSQSGFSRIIHYQKFNLLFSSFIKEEYLVPDDQRWDLIKNLCYQSPELENFRNYKLSDVVDRDMMVCTHGNIDVACSRFGYPIYKQLRQKYASKNLRIWRCSHFGGHQFAPTLIDFPNGQVWGHLESEVLDNLVRQEGQVKQLYKFYRGWVGVTKFAQIVEREIWTQRGWQWLNYQKSAQILNMDDNQHDPNWVEVQFDFISPDKVKGAYFARVEVNGSVMTARNSGDELISVKQYSVSYLKEIDK

SEQ ID NO: 76 - DNA, 1425 bp, Escherichia coli
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtag

SEQ ID NO: 77 - protein, 474 aa, Escherichia coli
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLA

SEQ ID NO: 78 - DNA, 746 bp, Escherichia coli
atgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaa

SEQ ID NO: 79 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSI

SEQ ID NO: 80 - DNA, 1500 bp, Synechocystis PCC6803
atgaattcatcccttgtgatcctttaccaccgtgagccctacgacgaagttagggaaaatggcaaaacggtgtatcgagagaaaaagagtcccaacgggattttgcccaccctcaaaagtttttttgccgatgcggaacagagcacctgggtcgcatggaaacaggtttcgccgaagcaaaaggatgattttcaggcggatatgtccattgaaggccttggcgatcgttgtacggtgcgccgggtgcccctgacggcggagcaggtaaaaaacttctatcacatcacttccaaggaagccttttggcccattctccactctttcccctggcagttcacctacgattcttctgattgggataattttcagcacattaaccgcttatttgccgaggcggcctgtgccgatgccgatgacaatgcattgttttgggtccacgactataacctctggttagcgcccctttacattcgtcagctcaagcccaacgccaagattgcctttttccaccacacccccttccccagcgttgatattttcaatattttgccctggcgggaggcgatcgtagaaagcttgctggcctgtgatctctgtggttttcatattccccgctacgtagaaaattttgtcgccgtggcccgtagtctcaagccggtggaaatcaccagacgggttgtggtagaccaagcctttaccccctacggtacggccctggcggaaccggaactcaccacccagttgcgttatggcgatcgcctcattaacctcgatgcgtttcccgtgggcaccaatccggcaaatatccgggcgatcgtggccaaagaaagtgtgcaacaaaaagttgctgaaattaaacaagatttaggcggtaagaggctaattgtttccgctgggcgggtggattacgtgaagggcaccaaggaaatgttgatgtgctatgaacgtctactggagcgtcgccccgaattgcagggggaaattagcctggtagtccccgtagccaaggccgctgagggaatgcgtatttatcgcaacgcccaaaacgaaattgaacgactggcagggaaaattaacggtcgctttgccaaactgtcctggacaccagtgatgctgttcacctctcctttagcctatgaggagctcattgccctgttctgtgccgccgacattgcctggatcactcccctgcgggatgggctaaacctggtggctaaggagtatgtggtggctaaaaatggcgaagaaggagttctgatcctctcggaatttgccggttgtgcggtggaactacccgatgcggtgttgactaacccctacgcttccagccgtatggacgaatccattgaccaggccctggccatggacaaagacgaacagaaaaaacgcatggggagaatgtacgccgccattaagcgttacgacgttcaacaatgggccaatcacctactgcgggaagcctacgccgatgtggtactgggagagcccccccaaatgtag

SEQ ID NO: 81 - protein, 499 aa, Synechocystis PCC6803
MNSSLVILYHREPYDEVRENGKTVYREKKSPNGILPTLKSFFADAEQSTWVAWKQVSPKQKDDFQADMSIEGLGDRCTVRRVPLTAEQVKNFYHITSKEAFWPILHSFPWQFTYDSSDWDNFQHINRLFAEAACADADDNALFWVHDYNLWLAPLYIRQLKPNAKIAFFHHTPFPSVDIFNILPWREAIVESLLACDLCGFHIPRYVENFVAVARSLKPVEITRRVVVDQAFTPYGTALAEPELTTQLRYGDRLINLDAFPVGTNPANIRAIVAKESVQQKVAEIKQDLGGKRLIVSAGRVDYVKGTKEMLMCYERLLERRPELQGEISLVVPVAKAAEGMRIYRNAQNEIERLAGKINGRFAKLSWTPVMLFTSPLAYEELIALFCAADIAWITPLRDGLNLVAKEYVVAKNGEEGVLILSEFAGCAVELPDAVLTNPYASSRMDESIDQALAMDKDEQKKRMGRMYAAIKRYDVQQWANHLLREAYADVVLGEPPQM

SEQ ID NO: 82 - DNA, 1269 bp, Synechocystis PCC6803
atggtattacaccaacaacgtttctccctcgaccatggagctttttgtcaaaccttagcccaaactgaaaatttactcattgtccaagacttggatggggtctgcatggaattagtgcaagatcccctcagtcgccgcctggatgccgattatgtccgggccaccaccctgtttgctgaacatttttacgtgttgaccaatggggagcacgtgggaaaaagaggagtacagggcattgtggaacaatcctttggggatgcttcctttgtgcaacaggaaggcctatatttgcccggtttggcggccgggggagtgcagtggcaggatcgccatggcaaagtaagtcatcctggagtggggcaaacggagctggagtttttagcggcggtgcccgaaaaaatcactaattgtttaaaaaccttttttggcgatcgcccccattccctatccccagagcaattacaaacgggcattgaagcttcggttttagataatgtggcttcccccaccgccaatttaaataccttggccaatctgttacaagactttccgcaaatttaccgagatttgcaggaaaccatggctcaattattggatcagttgatggcggaagccgttgcccagggtttggggaatagtttttttgtccactatgctcccaatttaggtagggatgaacgaggtaaggaaattattcgttgggccaaagctggggattccggcaccaccgattttcaatttatgttgcggggtggggtcaaagaagccggggttttggctttgctaaatcgttactatcacaatcggacagggcaatatcctctgggagaaagttttagtgctcgccaagcgcccccatcccaccaggacttgttgcatttggtgaaagcgcaatttgatccggccttgatgccgctgatcattggagttggggatacggtcaccagtcaggtggatgaagctaccggggaaattcgacgtggcgggagcgatcgccaatttttgcaattaatccaagatttgggggattggggaaatcacggtaacttagtggtgtatgtggacagttcccagggggaggtgaaaaatcgccaacctctacaactagaaaccgtggcggggcaaacccaagtggtggctggccctggggatatgcgggacagggaagagccattgaagatcaatgtggcttttcctggtggccatgaccaatatgtagcggcgtttaagcaggcggcccagcgccgaagagtccatttttcccagtag

SEQ ID NO: 83 - protein, 422 aa, Synechocystis PCC6803
MVLHQQRFSLDHGAFCQTLAQTENLLIVQDLDGVCMELVQDPLSRRLDADYVRATTLFAEHFYVLTNGEHVGKRGVQGIVEQSFGDASFVQQEGLYLPGLAAGGVQWQDRHGKVSHPGVGQTELEFLAAVPEKITNCLKTFFGDRPHSLSPEQLQTGIEASVLDNVASPTANLNTLANLLQDFPQIYRDLQETMAQLLDQLMAEAVAQGLGNSFFVHYAPNLGRDERGKEIIRWAKAGDSGTTDFQFMLRGGVKEAGVLALLNRYYHNRTGQYPLGESFSARQAPPSHQDLLHLVKAQFDPALMPLIIGVGDTVTSQVDEATGEIRRGGSDRQFLQLIQDLGDWGNHGNLVVYVDSSQGEVKNRQPLQLETVAGQTQVVAGPGDMRDREEPLKINVAFPGGHDQYVAAFKQAAQRRRVHFSQ

SEQ ID NO: 84 - DNA, 1365 bp, Agrobacterium tumefaciens
ttggaaaaatttaccaagatgggacccatgacaaccacgagcgaaactgaacgctatccgcggatagctctcatatcgacgcatggctatgtcgccgcacacccgcccctgggcgctgccgataccggggggcaggtggtttatgtgcttgagcttgcacgaaaactcggccaactcggttataccgtcgatctttacacccgacgcttcgaagaccagccggaattcgacgaggtcgatgagcgcgtccgtgtggtgcgcattccctgcggcgggcgcgatttcattcccaaggaatatctgcaccggcacctgatggaatggtgcgagaacgcgctacgcttcatcaaaaaaaacgacctcaattactccttcatcaacagccactactgggatgccggcgtggccgggcagcggctctccgaagcactgaaaatcccccatctgcacacgccgcactcgctcggcatctggaagaagcgccagatggagaccgattatccggaaaaggccgatacgttcgagcttgagttcaacttcaaggagcgcatccagcacgagctgatcatctatcgcagctgcgacatggtgatcgccaccacgccggtgcagctggacgtgctgatcgaagattatggcctgaagcgcaaacatatccacatgatcccgccgggttatgacgacaaccgcttcttccccgtctcggatgcgacgcgtcagatgatccggcagcgtttcggttttgaaggcaaagtggtgctggcactcggtcggctcgccaccaacaagggctacgacctgctgatcgacggcttttccgtgcttgccgagcgcgagccggaagcccgcctgcatctggccgtcggcggcgagaatatggacgagcaggaaaccaccattctcaaccagctgaaggagcgggtgaaatcgctcgggctggaagacaaggtggctttctctggttatgtcgcggacgaggatttgccggatatctatcgggctgccgatctcttcgtgctttccagccgctacgagcccttcggcatgaccgccatcgaggccatggcgagcggcacgccgaccgtcgtcaccatccatggcgggctgttccgcgccatcagctatgggcgacatgcgctgtttgccgatcctttcgacaaggaagatctcggcattaccatgatgaagccgttcaagcatgaacggctctacgggcggctttcgcgcatgggagcccacaaggcacgcagcctgttcacatggaccggaattgcccagcaacttctcgcgctcgtggaaggcaggaccatgatgccggttctggaagaagccgactgggccgaaccatggaatgacggcgattga

SEQ ID NO: 85 - protein, 454 aa, Agrobacterium tumefaciens
MEKFTKMGPMTTTSETERYPRIALISTHGYVAAHPPLGAADTGGQVVYVLELARKLGQLGYTVDLYTRRFEDQPEFDEVDERVRVVRIPCGGRDFIPKEYLHRHLMEWCENALRFIKKNDLNYSFINSHYWDAGVAGQRLSEALKIPHLHTPHSLGIWKKRQMETDYPEKADTFELEFNFKERIQHELIIYRSCDMVIATTPVQLDVLIEDYGLKRKHIHMIPPGYDDNRFFPVSDATRQMIRQRFGFEGKVVLALGRLATNKGYDLLIDGFSVLAEREPEARLHLAVGGENMDEQETTILNQLKERVKSLGLEDKVAFSGYVADEDLPDIYRAADLFVLSSRYEPFGMTAIEAMASGTPTVVTIHGGLFRAISYGRHALFADPFDKEDLGITMMKPFKHERLYGRLSRMGAHKARSLFTWTGIAQQLLALVEGRTMMPVLEEADWAEPWNDGD

SEQ ID NO: 86 - DNA, 747 bp, Agrobacterium tumefaciens
ttgaaaccgcttcgtcttctttccaccgatcttgacggaaccgtcgtcggcgataatgacgccacgcggcggttccgcgatttctggcacgcactgccggatgatcttcgcccggttctggtcttcaacagcggccggttgatcgacgatcagcttgcccttttggaagaggtgccgctgccgcagccggactacatcatcggcggtgtcggcaccatgctgcatgcaaaaaaacgcagcgaactggaaaccgcctatacacagtcgctcggcaccggttttgacccgcggaagattgccgatgtcatgaaccgcattgcgggcgtgacgatgcaggaggagcgttatcagcacggcctgaaatcgagctggttcctgcatgacgccgatgccgccgcgctcggcgagatcgaggccgcgcttctggccgccgatattgacgctcgtatcgtttattccagcgatcgcgacctcgacatattgccgaaggccgccgacaaaggcgcggcacttgcatggttgtgtggacaattgcgcatcggcctcgacgaatcagtggtctcgggtgatactggcaatgaccgtgcgatgtttgagttgaagactatccgcggcgtgatcgtgggcaatgccctgcctgagcttgtctcgctggcgcatcaggacaatcgcttttttcactcgaccgcgaaagaagcggatggcgtgatcgaaggcctgcggcactggggactgaacccccgctaa

SEQ ID NO: 87 - protein, 248 aa, Agrobacterium tumefaciens
MKPLRLLSTDLDGTVVGDNDATRRFRDFWHALPDDLRPVLVFNSGRLIDDQLALLEEVPLPQPDYIIGGVGTMLHAKKRSELETAYTQSLGTGFDPRKIADVMNRIAGVTMQEERYQHGLKSSWFLHDADAAALGEIEAALLAADIDARIVYSSDRDLDILPKAADKGAALAWLCGQLRIGLDESVVSGDTGNDRAMFELKTIRGVIVGNALPELVSLAHQDNRFFHSTAKEADGVIEGLRHWGLNPR

SEQ ID NO: 88 - DNA, 35 bp, Artificial Sequence
  PCR primer
tctcagggatcccataccatgattaaaaaaagtac

SEQ ID NO: 89 - DNA, 26 bp, Artificial Sequence
  PCR primer
ggccgtgagctcagaaccaggtttcc

SEQ ID NO: 90 - DNA, 1546 bp, Artificial Sequence
  PCR product
tctcagggatcccataccatgattaaaaaaagtacgcttgcccttacccttggcttaatggccggtactcccgccgcctttgccgacagcaatatgtccagcattgaggcgcgtctcgccgcgctggaacaacgtcttcaggcggctgaacagcgcgccagcgcggcggaaacccgcgctgaagccgcagagcgtcaggcacaggcgcttgccgcgcaacaaaaagcgcagccgccggttcagcctgtcgccgcgcaacctgcgccgcagcccgccacgcaaacggcggataacagcgggtttgaattccacggctacgcccgctcgggcctgctgatgaacgattccgccgcgaaaacgcagggcggcccgtccttcacgccagcgggtgaaaccggcggtcacgtcgggcgtctcggcaatgagccggacacttaccttgaaatgaacctagagcacaaacagacgctcgcgaacggcgccaccacgcgctttaaagtgatggtcgctgacggtcagcgcagctataacgactggacggcctccaccagcgatctcaacgtgcgccaggcgtttaccgaactcggccacctgccgaccttcatcggcgcgtttaaagatgccaccgtctgggccggtaaacgcttcgatcgtgataacttcgatatccactggattgactccgacgtggtgttcctcgccggtacgggtgcgggtatctacgacatgcgctggagcgataacgcccgcagtaacttctcgctgtatggccgcaccttcggcgatatcgaaaacagcgaaaacaccgcccagaactatatccttacgcttaataactacgtcgggccggtacagctgatggtgagcgggatgcgcgccaaagataacgaagaccgcgtggatatcgagggtaaccgcgtgaaaaaagacgcggcggaagatggcgtgcatgcgctgctcggcctgcataacgacagcttctacggtctgagcgacggctcctcgaaaaccgcactgctgtatggacatggcctgggcgcggaagtgaaatccatcggctccgatggcgcgctgctgccgcaggccgatacctggcgtctcgcgacctacggcatgacaccgctcggcggcggctggcatatcgcaccggcggtgctggcgcagagcagtaaagatcgctacgtcaaaggcgacagctaccagtgggcgaccgccaacctgcgcctcattcaggagattaaccagaactttgagctgcagtatgagggcagctatcagtacatggatctgcgcccgaaaggttacaacgaccgcaacgcggtcagcggcaacttctataagctgacctttgcgccgacgctgaaagcgggcgacgtgggcgaattcctcaagcgtcctgaactgcgcctgttcgccacctggatggactgggatcatcgcctggataactacgccagcaatgatgcctttggcagcaccggctttaccgccggcggtgaatggaacttcggcgtacagatggaaacctggttctgagctcacggcc

SEQ ID NO: 91 - DNA, 13332 bp, Artificial Sequence
  artifical plasmid pLybAL32 containing scrY
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcaggtcgactctagatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaattgttatcagctatgcgccgaccagaacaccttgccgatcagccaaacgtctcttcaggccactgactagcgataactttccccacaacggaacaactctcactgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggctttcttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatcttaagaaggaggatcccataccatgattaaaaaaagtacgcttgcccttacccttggcttaatggccggtactcccgccgcctttgccgacagcaatatgtccagcattgaggcgcgtctcgccgcgctggaacaacgtcttcaggcggctgaacagcgcgccagcgcggcggaaacccgcgctgaagccgcagagcgtcaggcacaggcgcttgccgcgcaacaaaaagcgcagccgccggttcagcctgtcgccgcgcaacctgcgccgcagcccgccacgcaaacggcggataacagcgggtttgaattccacggctacgcccgctcgggcctgctgatgaacgattccgccgcgaaaacgcagggcggcccgtccttcacgccagcgggtgaaaccggcggtcacgtcgggcgtctcggcaatgagccggacacttaccttgaaatgaacctagagcacaaacagacgctcgcgaacggcgccaccacgcgctttaaagtgatggtcgctgacggtcagcgcagctataacgactggacggcctccaccagcgatctcaacgtgcgccaggcgtttaccgaactcggccacctgccgaccttcatcggcgcgtttaaagatgccaccgtctgggccggtaaacgcttcgatcgtgataacttcgatatccactggattgactccgacgtggtgttcctcgccggtacgggtgcgggtatctacgacatgcgctggagcgataacgcccgcagtaacttctcgctgtatggccgcaccttcggcgatatcgaaaacagcgaaaacaccgcccagaactatatccttacgcttaataactacgtcgggccggtacagctgatggtgagcgggatgcgcgccaaagataacgaagaccgcgtggatatcgagggtaaccgcgtgaaaaaagacgcggcggaagatggcgtgcatgcgctgctcggcctgcataacgacagcttctacggtctgagcgacggctcctcgaaaaccgcactgctgtatggacatggcctgggcgcggaagtgaaatccatcggctccgatggcgcgctgctgccgcaggccgatacctggcgtctcgcgacctacggcatgacaccgctcggcggcggctggcatatcgcaccggcggtgctggcgcagagcagtaaagatcgctacgtcaaaggcgacagctaccagtgggcgaccgccaacctgcgcctcattcaggagattaaccagaactttgagctgcagtatgagggcagctatcagtacatggatctgcgcccgaaaggttacaacgaccgcaacgcggtcagcggcaacttctataagctgacctttgcgccgacgctgaaagcgggcgacgtgggcgaattcctcaagcgtcctgaactgcgcctgttcgccacctggatggactgggatcatcgcctggataactacgccagcaatgatgcctttggcagcaccggctttaccgccggcggtgaatggaacttcggcgtacagatggaaacctggttctgagctcgaattggggcgttttctgtgaggctgactagcgcgtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 92 - DNA, 21 bp, Artificial Sequence
  PCR primer
gcagtaacttctcgctgtatg

SEQ ID NO: 93 - DNA, 23 bp, Artificial Sequence
  PCR primer
gtgttttcgctgttttcgatatc

SEQ ID NO: 94 - DNA, 1518 bp, Enterobacter sakazakii
atgattaaaaaaagtacgcttgcccttacccttggcttaatggccggtactcccgccgcctttgccgacagcaatatgtccagcattgaggcgcgtctcgccgcgctggaacaacgtcttcaggcggctgaacagcgcgccagcgcggcggaaacccgcgctgaagccgcagagcgtcaggcacaggcgcttgccgcgcaacaaaaagcgcagccgccggttcagcctgtcgccgcgcaacctgcgccgcagcccgccacgcaaacggcggataacagcgggtttgaattccacggctacgcccgctcgggcctgctgatgaacgattccgccgcgaaaacgcagggcggcccgtccttcacgccagcgggtgaaaccggcggtcacgtcgggcgtctcggcaatgagccggacacttaccttgaaatgaacctagagcacaaacagacgctcgcgaacggcgccaccacgcgctttaaagtgatggtcgctgacggtcagcgcagctataacgactggacggcctccaccagcgatctcaacgtgcgccaggcgtttaccgaactcggccacctgccgaccttcatcggcgcgtttaaagatgccaccgtctgggccggtaaacgcttcgatcgtgataacttcgatatccactggattgactccgacgtggtgttcctcgccggtacgggtgcgggtatctacgacatgcgctggagcgataacgcccgcagtaacttctcgctgtatggccgcaccttcggcgatatcgaaaacagcgaaaacaccgcccagaactatatccttacgcttaataactacgtcgggccggtacagctgatggtgagcgggatgcgcgccaaagataacgaagaccgcgtggatatcgagggtaaccgcgtgaaaaaagacgcggcggaagatggcgtgcatgcgctgctcggcctgcataacgacagcttctacggtctgagcgacggctcctcgaaaaccgcactgctgtatggacatggcctgggcgcggaagtgaaatccatcggctccgatggcgcgctgctgccgcaggccgatacctggcgtctcgcgacctacggcatgacaccgctcggcggcggctggcatatcgcaccggcggtgctggcgcagagcagtaaagatcgctacgtcaaaggcgacagctaccagtgggcgaccgccaacctgcgcctcattcaggagattaaccagaactttgagctgcagtatgagggcagctatcagtacatggatctgcgcccgaaaggttacaacgaccgcaacgcggtcagcggcaacttctataagctgacctttgcgccgacgctgaaagcgggcgacgtgggcgaattcctcaagcgtcctgaactgcgcctgttcgccacctggatggactgggatcatcgcctggataactacgccagcaatgatgcctttggcagcaccggctttaccgccggcggtgaatggaacttcggcgtacagatggaaacctggttctga

SEQ ID NO: 95 - protein, 505 aa, Enterobacter sakazakii
MIKKSTLALTLGLMAGTPAAFADSNMSSIEARLAALEQRLQAAEQRASAAETRAEAAERQAQALAAQQKAQPPVQPVAAQPAPQPATQTADNSGFEFHGYARSGLLMNDSAAKTQGGPSFTPAGETGGHVGRLGNEPDTYLEMNLEHKQTLANGATTRFKVMVADGQRSYNDWTASTSDLNVRQAFTELGHLPTFIGAFKDATVWAGKRFDRDNFDIHWIDSDVVFLAGTGAGIYDMRWSDNARSNFSLYGRTFGDIENSENTAQNYILTLNNYVGPVQLMVSGMRAKDNEDRVDIEGNRVKKDAAEDGVHALLGLHNDSFYGLSDGSSKTALLYGHGLGAEVKSIGSDGALLPQADTWRLATYGMTPLGGGWHIAPAVLAQSSKDRYVKGDSYQWATANLRLIQEINQNFELQYEGSYQYMDLRPKGYNDRNAVSGNFYKLTFAPTLKAGDVGEFLKRPELRLFATWMDWDHRLDNYASNDAFGSTGFTAGGEWNFGVQMETWF

SEQ ID NO: 96 - DNA, 30 bp, Artificial Sequence
  PCR primer
ccacaatggactgccagccgtcaaaggatg

SEQ ID NO: 97 - DNA, 30 bp, Artificial Sequence
  PCR primer
gcccaactggtcacggacatcgtcgataac

SEQ ID NO: 98 - DNA, 29 bp, Artificial Sequence
  PCR primer
tgcaatggctccaggaagcccgatcgatg

SEQ ID NO: 99 - DNA, 30 bp, Artificial Sequence
  PCR primer
ggcagcattacggctcagaccttggtcatg

SEQ ID NO: 100 - DNA, 1246 bp, Synechocystis PCC6803
ccacaatggactgccagccgtcaaaggatggttgtttgctcataatgcttgcctgtctgtcgttgaacttgggggaaatccctgcccaaagtatggcagaaaacctttcccttcccaatgccccaacttccggtaacccgatctgagctacagtggagttccgcggtgaattgttaccgacggtgagaccacgtcctaacttttagcccatttttcggttccccaacggccaagattaacaaaattaaattttagatattaacttttaagttttcccatggcttctcaattacgtgtttatgtgccggagcatcctctaattaagcattggttgggggtagctagggatgaaaacacgccgccggttttgtttaaaactgccatgggggaattgggacgttggttgacctatgaggccgctcgttattggttgccgacggtggatacggaagtgaaaactcccctggcgatcgccaaggccagtcttattgacccccaaacgccctttgtcattgtgcccattttgcgggcggggttggctctggtggaaggggcccaggggttgttgcccctggcaaaaatttaccatctgggtttagtgcgcaatgaaactaccctggaacctagtctgtatctgaacaagttgccggagcggtttgcccccggtacccatcttttgttgctagatcccatgttggctacgggtaataccatcatggctgctttggatttgctgatggcccgggacattgatgccaatttaatccgtttggtctccgtggtggccgcccccactgccctgcaaaaattaagtaatgcccatcccaatttgaccatctacaccgccatgattgacgaacaactcaatgaccggggttacattgtgcccggcctaggggatgcaggcgatcgttgctttggtacttgataacaccattaaactagtgatcaaataattacaaattcacccccaaacgttaacaacaggagtaaagtcatggctcaaaaagataacttcgccggaggatttttattaggtacggtcattggtggcgtagtggggggaattttgggttctgtcctggccaatcgagctgctacccaaagccccgaccgggaaaaattagacactgagggggtaggaaatctcgatagtgaggaaaatattgagttggctcgccgtcgcctggaagacaaaattgcccaacttaatttggttatcgacgatgtccgtgaccagttgggc

SEQ ID NO: 101 - DNA, 1281 bp, Synechococcus PCC7942
tgcaatggctccaggaagcccgatcgatgggatttcaagtcgctttagatgattttgggacgggttattccagccttggttacctcaagcgtttgcccatcaatgctctcaaaattgatcgcagctttattcgcgatctgccgcacgaccatgacgatcaagcgatcgtgcaggcgattgttgcaatggccaaggtcttgaaacttcgcacgatcgcagaaggcgtagaacgcctcgagcaagccgccttcttagaagcgattggttgtgatgctgtgcaagggttcttctatggcccaccactgcccgaagcagaagcgcttgccttcctgcaccgttccgcttcccctggggtctgaacgttaaaatcaggagctgtcttctgctgattggcatggctcctcaactgcgtatcttcgtgccgccccatcccttaattcggcactggctgggcattgcccgcgatcgccagacgccgacgcctctgtttcgcaccgcgatcgcagagctgggccgctggctcgcctatgaggctgtgcgggaatggctaccaacgattccagcggcggtgcaaactcctcttgcagaaaccccagcggagttcgtcgatttttcgcaacccttggcgatcgtgccgattctgcgcgcaggtctgggtttagtggagtctgtccaacaggttttgccgactgcccgcatttttcacgtgggtctcaagcgggatgaagtcagtcttgaaccgcgctgctacctcaatcacctgccagagcaacttgaagtgaacagtcgcgttctggttctcgacccgatgctggcgacaggtggctcgctgctctatacccttgatttgctgcgcgatcgcggtgtctctgctgagcaagtgcgggtgctttcaattgtggctgccccgccagcgctacaaaaactcagtcaagcctacccggcgttgacgatttacagcgccatcattgatgagcagctgaacgacaaaggctttatcgtgccggggctgggggatgctggcgatcgcctgtttggtactccttgatctgctgactgaattcgctaggcttcagcgttgagcaaagcctgaacggcctgccgaatgaagctttcatcctgcggattttggctggggttgcccgcgcggtgaccccagatcgagggaattgggcaatagtgcgccttaggaatcaactgcgcttcggcctcacaatcctctggggtgaagtagagatctgttgtcgagggcatgaccaaggtctgagccgtaatgctgcc

SEQ ID NO: 102 - DNA, 9385 bp, Artificial Sequence
  plasmid pLybAL3f containing Synechocystis upp gene
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacgcccaactggtcacggacatcgtcgataaccaaattaagttgggcaattttgtcttccaggcgacggcgagccaactcaatattttcctcactatcgagatttcctaccccctcagtgtctaatttttcccggtcggggctttgggtagcagctcgattggccaggacagaacccaaaattccccccactacgccaccaatgaccgtacctaataaaaatcctccggcgaagttatctttttgagccatgactttactcctgttgttaacgtttgggggtgaatttgtaattatttgatcactagtttaatggtgttatcaagtaccaaagcaacgatcgcctgcatcccctaggccgggcacaatgtaaccccggtcattgagttgttcgtcaatcatggcggtgtagatggtcaaattgggatgggcattacttaatttttgcagggcagtgggggcggccaccacggagaccaaacggattaaattggcatcaatgtcccgggccatcagcaaatccaaagcagccatgatggtattacccgtagccaacatgggatctagcaacaaaagatgggtaccgggggcaaaccgctccggcaacttgttcagatacagactaggttccagggtagtttcattgcgcactaaacccagatggtaaatttttgccaggggcaacaacccctgggccccttccaccagagccaaccccgcccgcaaaatgggcacaatgacaaagggcgtttgggggtcaataagactggccttggcgatcgccaggggagttttcacttccgtatccaccgtcggcaaccaataacgagcggcctcataggtcaaccaacgtcccaattcccccatggcagttttaaacaaaaccggcggcgtgttttcatccctagctacccccaaccaatgcttaattagaggatgctccggcacataaacacgtaattgagaagccatgggaaaacttaaaagttaatatctaaaatttaattttgttaatcttggccgttggggaaccgaaaaatgggctaaaagttaggacgtggtctcaccgtcggtaacaattcaccgcggaactccactgtagctcagatcgggttaccggaagttggggcattgggaagggaaaggttttctgccatactttgggcagggatttcccccaagttcaacgacagacaggcaagcattatgagcaaacaaccatcctttgacggctggcagtccattgtgggtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 103 - DNA, 9420 bp, Artificial Sequence
  plasmid pLybAL5f containing Synechococcus upp gene
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacggcagcattacggctcagaccttggtcatgccctcgacaacagatctctacttcaccccagaggattgtgaggccgaagcgcagttgattcctaaggcgcactattgcccaattccctcgatctggggtcaccgcgcgggcaaccccagccaaaatccgcaggatgaaagcttcattcggcaggccgttcaggctttgctcaacgctgaagcctagcgaattcagtcagcagatcaaggagtaccaaacaggcgatcgccagcatcccccagccccggcacgataaagcctttgtcgttcagctgctcatcaatgatggcgctgtaaatcgtcaacgccgggtaggcttgactgagtttttgtagcgctggcggggcagccacaattgaaagcacccgcacttgctcagcagagacaccgcgatcgcgcagcaaatcaagggtatagagcagcgagccacctgtcgccagcatcgggtcgagaaccagaacgcgactgttcacttcaagttgctctggcaggtgattgaggtagcagcgcggttcaagactgacttcatcccgcttgagacccacgtgaaaaatgcgggcagtcggcaaaacctgttggacagactccactaaacccagacctgcgcgcagaatcggcacgatcgccaagggttgcgaaaaatcgacgaactccgctggggtttctgcaagaggagtttgcaccgccgctggaatcgttggtagccattcccgcacagcctcataggcgagccagcggcccagctctgcgatcgcggtgcgaaacagaggcgtcggcgtctggcgatcgcgggcaatgcccagccagtgccgaattaagggatggggcggcacgaagatacgcagttgaggagccatgccaatcagcagaagacagctcctgattttaacgttcagaccccaggggaagcggaacggtgcaggaaggcaagcgcttctgcttcgggcagtggtgggccatagaagaacccttgcacagcatcacaaccaatcgcttctaagaaggcggcttgctcgaggcgttctacgccttctgcgatcgtgcgaagtttcaagaccttggccattgcaacaatcgcctgcacgatcgcttgatcgtcatggtcgtgcggcagatcgcgaataaagctgcgatcaattttgagagcattgatgggcaaacgcttgaggtaaccaaggctggaataacccgtcccaaaatcatctaaagcgacttgaaatcccatcgatcgggcttcctggagccattgcagtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 104 - DNA, 22 bp, Artificial Sequence
  PCR primer
gtaatacgactcactatagggc

SEQ ID NO: 105 - DNA, 24 bp, Artificial Sequence
  PCR primer
cacacaggaaacagctatgaccat

SEQ ID NO: 106 - DNA, 5641 bp, Artificial Sequence
  plasmid pLybAL4f containing Synechocystis upp gene
tgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacgcccaactggtcacggacatcgtcgataaccaaattaagttgggcaattttgtcttccaggcgacggcgagccaactcaatattttcctcactatcgagatttcctaccccctcagtgtctaatttttcccggtcggggctttgggtagcagctcgattggccaggacagaacccaaaattccccccactacgccaccaatgaccgtacctaataaaaatcctccggcgaagttatctttttgagccatgactttactcctgttgttaacgtttgggggtgaatttgtaattatttgatcactagtttaatggtgttatcaagtaccaaagcaacgatcgcctgcatcccctaggccgggcacaatgtaaccccggtcattgagttgttcgtcaatcatggcggtgtagatggtcaaattgggatgggcattacttaatttttgcagggcagtgggggcggccaccacggagaccaaacggattaaattggcatcaatgtcccgggccatcagcaaatccaaagcagccatgatggtattacccgtagccaacatgggatctagcaacaaaagatgggtaccgggggcaaaccgctccggcaacttgttcagatacagactaggttccagggtagtttcattgcgcactaaacccagatggtaaatttttgccaggggcaacaacccctgggccccttccaccagagccaaccccgcccgcaaaatgggcacaatgacaaagggcgtttgggggtcaataagactggccttggcgatcgccaggggagttttcacttccgtatccaccgtcggcaaccaataacgagcggcctcataggtcaaccaacgtcccaattcccccatggcagttttaaacaaaaccggcggcgtgttttcatccctagctacccccaaccaatgcttaattagaggatgctccggcacataaacacgtaattgagaagccatgggaaaacttaaaagttaatatctaaaatttaattttgttaatcttggccgttggggaaccgaaaaatgggctaaaagttaggacgtggtctcaccgtcggtaacaattcaccgcggaactccactgtagctcagatcgggttaccggaagttggggcattgggaagggaaaggttttctgccatactttgggcagggatttcccccaagttcaacgacagacaggcaagcattatgagcaaacaaccatcctttgacggctggcagtccattgtgggtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctga

SEQ ID NO: 107 - DNA, 5218 bp, Artificial Sequence
  plasmid pLybAL9f containing partially deleted Synechocystis upp gene
tgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacgcccaactggtcacggacatcgtcgataaccaaattaagttgggcaattttgtcttccaggcgacggcgagccaactcaatattttcctcactatcgagatttcctaccccctcagtgtctaatttttcccggtcggggctttgggtagcagctcgattggccaggacagaacccaaaattccccccactacgccaccaatgaccgtacctaataaaaatcctccggcgaagttatctttttgagccatgactttactcctgttgttaacgtttgggggtgaatttgtaattatttgatcactagtttaatggtgttatcaagtaccaaagcaacgatcgcctgcatcccctagcgccaggggagttttcacttccgtatccaccgtcggcaaccaataacgagcggcctcataggtcaaccaacgtcccaattcccccatggcagttttaaacaaaaccggcggcgtgttttcatccctagctacccccaaccaatgcttaattagaggatgctccggcacataaacacgtaattgagaagccatgggaaaacttaaaagttaatatctaaaatttaattttgttaatcttggccgttggggaaccgaaaaatgggctaaaagttaggacgtggtctcaccgtcggtaacaattcaccgcggaactccactgtagctcagatcgggttaccggaagttggggcattgggaagggaaaggttttctgccatactttgggcagggatttcccccaagttcaacgacagacaggcaagcattatgagcaaacaaccatcctttgacggctggcagtccattgtgggtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctga

SEQ ID NO: 108 - DNA, 880 bp, Artificial Sequence
  sequence of partially deleted Synechocystis upp
gagctcggtacccggggatcccacgcccaactggtcacggacatcgtcgataaccaaattaagttgggcaattttgtcttccaggcgacggcgagccaactcaatattttcctcactatcgagatttcctaccccctcagtgtctaatttttcccggtcggggctttgggtagcagctcgattggccaggacagaacccaaaattccccccactacgccaccaatgaccgtacctaataaaaatcctccggcgaagttatctttttgagccatgactttactcctgttgttaacgtttgggggtgaatttgtaattatttgatcactagtttaatggtgttatcaagtaccaaagcaacgatcgcctgcatcccctagcgccaggggagttttcacttccgtatccaccgtcggcaaccaataacgagcggcctcataggtcaaccaacgtcccaattcccccatggcagttttaaacaaaaccggcggcgtgttttcatccctagctacccccaaccaatgcttaattagaggatgctccggcacataaacacgtaattgagaagccatgggaaaacttaaaagttaatatctaaaatttaattttgttaatcttggccgttggggaaccgaaaaatgggctaaaagttaggacgtggtctcaccgtcggtaacaattcaccgcggaactccactgtagctcagatcgggttaccggaagttggggcattgggaagggaaaggttttctgccatactttgggcagggatttcccccaagttcaacgacagacaggcaagcattatgagcaaacaaccatcctttgacggctggcagtccattgtgggtgggatcctctagagtcgacctgcaggcatgc

SEQ ID NO: 109 - DNA, 5800 bp, Artificial Sequence
  plasmid pLybAL6fb containing Synechococcus upp gene
tgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacggcagcattacggctcagaccttggtcatgccctcgacaacagatctctacttcaccccagaggattgtgaggccgaagcgcagttgattcctaaggcgcactattgcccaattccctcgatctggggtcaccgcgcgggcaaccccagccaaaatccgcaggatgaaagcttcattcggcaggccgttcaggctttgctcaacgctgaagcctagcgaattcagtcagcagatcaaggagtaccaaacaggcgatcgccagcatcccccagccccggcacgataaagcctttgtcgttcagctgctcatcaatgatggcgctgtaaatcgtcaacgccgggtaggcttgactgagtttttgtagcgctggcggggcagccacaattgaaagcacccgcacttgctcagcagagacaccgcgatcgcgcagcaaatcaagggtatagagcagcgagccacctgtcgccagcatcgggtcgagaaccagaacgcgactgttcacttcaagttgctctggcaggtgattgaggtagcagcgcggttcaagactgacttcatcccgcttgagacccacgtgaaaaatgcgggcagtcggcaaaacctgttggacagactccactaaacccagacctgcgcgcagaatcggcacgatcgccaagggttgcgaaaaatcgacgaactccgctggggtttctgcaagaggagtttgcaccgccgctggaatcgttggtagccattcccgcacagcctcataggcgagccagcggcccagctctgcgatcgcggtgcgaaacagaggcgtcggcgtctggcgatcgcgggcaatgcccagccagtgccgaattaagggatggggcggcacgaagatacgcagttgaggagccatgccaatcagcagaagacagctcctgattttaacgttcagaccccaggggaagcggaacggtgcaggaaggcaagcgcttctgcttcgggcagtggtgggccatagaagaacccttgcacagcatcacaaccaatcgcttctaagaaggcggcttgctcgaggcgttctacgccttctgcgatcgtgcgaagtttcaagaccttggccattgcaacaatcgcctgcacgatcgcttgatcgtcatggtcgtgcggcagatcgcgaataaagctgcgatcaattttgagagcattgatgggcaaacgcttgaggtaaccaaggctggaataacccgtcccaaaatcatctaaagcgacttgaaatcccatcgatcgggcttcctggagccattgcagtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttat

SEQ ID NO: 110 - DNA, 5731 bp, Artificial Sequence
  plasmid pLybAL10fb containing partially deleted Synechococcus upp gene
tgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacggcagcattacggctcagaccttggtcatgccctcgacaacagatctctacttcaccccagaggattgtgaggccgaagcgcagttgattcctaaggcgcactattgcccaattccctcgatctggggtcaccgcgcgggcaaccccagccaaaatccgcaggatgaaagcttcattcggcaggccgttcaggctttgctcaacgctgaagcctagcgaattcagtcagcagatcaaggagtaccaaacaggcgatcgccagcatcccccagccccggcacgataaagcctttgtcgttcagctgctcatcaatgatggcgctgtaaatcgtcaacgccgggtaggcttgactgagtttttgtagcgctggcggggcagccacaattgaaagcacccgcacttgctcagcagagacaccgcgatcgcgcagcaaatcaagggtatagagcagcgagccacctgtcgccagcatcgggtcgagaaccagaacgcgactgttcacttcaagttgctctggcaggtgattgaggtagcagcgcggttcaagactgacttcatcccgctcgcgcagaatcggcacgatcgccaagggttgcgaaaaatcgacgaactccgctggggtttctgcaagaggagtttgcaccgccgctggaatcgttggtagccattcccgcacagcctcataggcgagccagcggcccagctctgcgatcgcggtgcgaaacagaggcgtcggcgtctggcgatcgcgggcaatgcccagccagtgccgaattaagggatggggcggcacgaagatacgcagttgaggagccatgccaatcagcagaagacagctcctgattttaacgttcagaccccaggggaagcggaacggtgcaggaaggcaagcgcttctgcttcgggcagtggtgggccatagaagaacccttgcacagcatcacaaccaatcgcttctaagaaggcggcttgctcgaggcgttctacgccttctgcgatcgtgcgaagtttcaagaccttggccattgcaacaatcgcctgcacgatcgcttgatcgtcatggtcgtgcggcagatcgcgaataaagctgcgatcaattttgagagcattgatgggcaaacgcttgaggtaaccaaggctggaataacccgtcccaaaatcatctaaagcgacttgaaatcccatcgatcgggcttcctggagccattgcagtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttat

SEQ ID NO: 111 - DNA, 651 bp, Synechocystis PCC6803; identical to SEQ ID NO: 56

SEQ ID NO: 112 - protein, 216 aa, Synechocystis PCC6803; identical to SEQ ID NO: 57

SEQ ID NO: 113 - DNA, 654 bp, Synechococcus PCC7942; identical to SEQ ID NO: 58

SEQ ID NO: 114 - protein, 217 aa, Synechococcus PCC7942; identical to SEQ ID NO: 59

SEQ ID NO: 115 - DNA, 2184 bp, Artificial Sequence
  PCR product
ttgcatcttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagcaatctc

SEQ ID NO: 116 - DNA, 46 bp, Artificial Sequence
  PCR primer
ttgcatcttaagaaggaggatccatatgatcttgatggaacgctgg

SEQ ID NO: 117 - DNA, 28 bp, Artificial Sequence
  PCR primer
gagattgctagctcctacgcaagctttg

SEQ ID NO: 118 - DNA, 12051 bp, Artificial Sequence
  plasmid pLybAL23 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcaggtcgactctagatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaattgttatcagctatgcgccgaccagaacaccttgccgatcagccaaacgtctcttcaggccactgactagcgataactttccccacaacggaacaactctcactgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggctttcttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatcttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 119 - DNA, 23 bp, Artificial Sequence
  PCR primer
ttcattatcggctcgatctggtg

SEQ ID NO: 120 - DNA, 25 bp, Artificial Sequence
  PCR primer
caacaggtgataatcgtggatccag

SEQ ID NO: 121 - DNA, 11348 bp, Artificial Sequence
  plasmid pLybAL28 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgccgttattgatggaatgggaagaagcaatggtcacaataaactggaggttatgggtatgttttttagccctaatgctccaatcgccttgattgtatcgaatgatgcagtctctaaaattgtatccgtaaaagacctctgcaccgccgacgggtctggattatgggcaataatcacagtcgagccagactacccctggaggtaaactccggggctggagccataaagattaggaattcattaagaaatgtaacaatcgacgttctagatcataccacgcccccactgtccggcagggtgaacagaggagactttcccctgttacagtgtcagtgacaaaacaactttttggcatcggtgcaggtggtgagccatggcggcccagatcattgaaattctttccccggaggaaatccgacgtacccttacccgtctggcttcccaggtaatttaggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 122 - DNA, 11527 bp, Artificial Sequence
  plasmid pLybAL29 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgccgagcctgatgtgtgacacctaagatcactccagttctctttggaaactggctgatgagtgaagacaccatctttggcaagatcatccggcgcgagattccagcagacattgtttatgaagatgatctctgtctggcttttcgagatgtggcaccccaagcgccggttcacattctggtgattcccaagcaaccaattgccaaccttttggaagcgacagcagaacatcaagcgctgctgggtcatttgttgctgactgtaaaggcgatcgcggcccaagaaggactcaccgagggctaccgcaccgtgattaacacgggccctgcgggtgggcaaaccgtttaccacctgcatattcacttactgggcgggcgatcgctggcttggccgcccggctgagaaaagtctgaaagttctttacaaaactcaatctgcttgttagattttactcacgaggctattaagtctcgtaaatagttcaactaaggactcatcgcaaaatgacgactgcattgcagcggcgcgagagcgccagcctgtggcagcagttctgcgagtgggtaaccagcaccgacaaccgcctctatgtgggttggttcggcgtgctgatgatccccactctgctgaccggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 123 - DNA, 11769 bp, Artificial Sequence
  plasmid pLybAL30 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcaccagtaaacataaatctccccggcgacgcaaaaaacgggtgaccatcaagccggtgcgcttcggcatttttctgctttgcctagcaggcattgtggggggggcaactgccctaattatcaatcgtactggcgatcccctaggtgggttgctagaagaccccctagatgttttcctggaccaaccttcagaatttatccccgatgaagccacgagccggaatttgattctcagtcaacccaacttcaatcagcaagtgggtcagatggtagtacaaggctggcttgatagtaaaaagttagcctttggccaaaactacgatgtcggggcattgcagagtgttttagcccccaatctccttgcccaacaacggggtcgggcccaacgggatcaagcccaaaaggtctatcaccaatacgaacacaagttgcagattttagcctatcaagttaacccccaagaccccaaccgagccaccgttactgcccgggtagaagaaattagccagccctttaccctaggtaatcaacagcagaagggctccgccaccaaagatgacttgactgtgcgctatcagctagtacgacaccaaggggtttggaaaattgaccaaatacaagtggtaaatggcccccgttagtgcgtggcgttaactccccttttgaccaatggcatacggctagatgcccccataggtacggaaacctgcacttccgagaactaagcccctaccgtcactataagagtgtgaacgtgtcggccccaggcaatggattggaaccatggcttttcggcccatcgttgtgtcttatattcttacttgttaacgggagttaattaaaattatgggaaaagttgttgggattgacctcggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 124 - DNA, 11477 bp, Artificial Sequence
  plasmid pLybAL31 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcaaagctcactaactgggcgggattttccgggtccggttgctgacggtaatagtcgtctaaaagtttggccacatccaaaaggctgtcggcggggggatgctggccggcgaggggattaattctgcttgtcatatacaaaaattgtaaaaaatggagggcggcgatcaggggcttagacacccaaatcctagccaaaaagggttaactagccaagggctatccatgggcaaagagataaaagaaaaagtctccaaatccctggtcatagagaaaaaattgccaaagttaccccaggccatacacggcccagcgccaagatggggagcacaaattcaaactttgtaaacaggccggaagctatccggccaaggagcactcagattgtgttaacgttcaggggagttgcttaacacaattttccaattaatagtattaatattttcttaacttgcaccgtaccatggtgagaaagcctatctgagcccttatttgattaaccttcgactgattattgatcccctgtgcagtctcccctctccctctgtctttttgctcccgaacacgttgcccatagactcaggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 125 - DNA, 11258 bp, Artificial Sequence
  plasmid pLybAL36 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcaggaaaacaagctcagaatgctgcggggagaagggcaactccccaccagccccaaatttttgctggcgataaatatttttcggtttaattgttcacaaagctttttgaatttgagtttatagaaatttattggctggtaatgcttttttgcccccctgcaggacttcattgatccttgcctataccatcaatatcattggtcaataatgatgatgattgactaaaacatgtttaacaaaatttaacgcatatgctaaatgcgtaaactgcatatgccttggctgagtgtaatttacgttacaaattttaacgaaacgggaaccctatattgatctctactgttatctggcttgaagcgttggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 126 - DNA, 11453 bp, Artificial Sequence
  plasmid pLybAL37 containing otsBA operon
aggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcataaatttctgttttgaccaaaccatcccgacataactcggtcagggcttgcaaaacagcggggatgcgatcgtgctgccagagactgcaaaggtgagccaataaccactgcgtctgccagtcatcaggtatcgcttggcagcgctgcaacccagcttcgaggacgcgaacatcaactgttttggccagttgctgaacctgtcgccaacaatgttcaaaatcaccgcttggccagccgtcactctctgcaaacgctgcatcagtcatgtgcaatcaatacaggttaaaaaccatgctaatggctccacctaagcgggcttcagagtcaaggcttgtagcaattgctactaaaaactgcgatcgctgctgaaatgagctggaattctgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcaagctgctttgtaggcagatgaatccatggtaccgttaagaaggaggatccatatgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtaggagctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaaccttttcagaatgagacgttgatcggcacgtaagcgtgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacacaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaa

SEQ ID NO: 127 - DNA, 1754 bp, Artificial Sequence
  PCR product
tattcgcttaagccaaaggagaatgattgatgaaatcccccgcaccttctcgcccgcaaaaaatggcgttaattccagcctgtatctttttgtgtttcgctgcgctatcggtgcaggcagaagaaacaccggtaacaccacagccgcctgatattttattagggccgctgtttaatgatgtgcaaaacgccaaactttttccggaccaaaaaacctttgccgatgccgtgccgaacagcgatccgctgatgatccttgctgattatcggatgcagcaaaaccagagcggatttgatctgcgccatttcgttaacgtcaatttcaccctgccgaaagaaggcgagaaatatgttccgccagaggggcagtcactgcgcgaacatattgacggactttggccggtattaacgcgttctaccgaaaacaccgaaaaatgggattctctgttaccgctgccggaaccttatgtcgtgccgggcggacgctttcgcgaggtatattactgggacagttacttcaccatgttaggacttgccgaaagcggtcactgggataaagtcgcggatatggtggccaattttgctcatgaaatagacacttacggtcatattcccaacggcaaccgcagttactatttaagccgctcgcaaccgcccttctttgccctgatggtagagttactggcgcagcatgaaggcgatgccgcgttgaagcaatacctgccgcaaatgcaaaaagaatatgcttactggatggacggtgttgaaaacctgcaagccggacaacaggaaaaacgcgttgtcaaacttcaggatggtacccttctcaaccgctactgggacgatcgcgatacgccacgaccagagtcatgggtggaagatattgccaccgccaaaagcaatccgaatcgacctgccactgaaatttaccgcgacctgcgctctgccgctgcgtctggctgggatttcagctcgcgctggatggacaacccgcagcagttaaataccttacgcaccaccagcatcgtaccggtcgatctgaacagcctgatgtttaaaatggaaaaaatcctcgcccgcgccagcaaagctgccggagataacgcgatggcaaaccagtacgaaacgctggcaaatgcccgtcaaaaagggatcgaaaaatacctgtggaacgatcaacaaggctggtatgccgattacgacctgaaaagtcataaagtgcgcaatcagttaaccgcggccgccctgttcccgctgtacgtcaatgcggcagcgaaagatcgcgccaacaaaatggcgacggcgacgaaaacacatctgctgcaacccggcggcctgaacaccacgtcggtgaaaagtgggcaacaatgggatgcgccaaatggctgggcaccgttacagtgggtcgcgacagaaggattacaaaactacgggcaaaaagaggtggcgatggacattagctggcacttcctgaccaatgttcagcacacctatgaccgggagaaaaagctggtggaaaaatatgatgtcagcaccaccggaacggggggcggcggtggcgaatatccattacaggatggctttggctggaccaatggcgtgacgctgaaaatgctggatttgatctgcccgaaagagcaaccgtgtgacaatgttccggcgacgcgtccgaccgttaagtcagcaacgacgcaaccctcaaccaaagaggcacaacccacaccttaaccagcgcttactccgtctagatcattc

SEQ ID NO: 128 - DNA, 29 bp, Artificial Sequence
  PCR primer
tattcgcttaagccaaaggagaatgattg

SEQ ID NO: 129 - DNA, 27 bp, Artificial Sequence
  PCR primer
gaatgatctagacggagtaagcgctgg

SEQ ID NO: 130 - DNA, 6282 bp, Artificial Sequence
  plasmid pLybAL24 containing treA
tagtcatgccccgcgcccaccggaaggagctgactgggttgaaggctctcaagggcatcggtcgataaatattctgaaatgagctgttgacaattaatcatcgaactagttaacttttacgcaagttcttaagccaaaggagaatgattgatgaaatcccccgcaccttctcgcccgcaaaaaatggcgttaattccagcctgtatctttttgtgtttcgctgcgctatcggtgcaggcagaagaaacaccggtaacaccacagccgcctgatattttattagggccgctgtttaatgatgtgcaaaacgccaaactttttccggaccaaaaaacctttgccgatgccgtgccgaacagcgatccgctgatgatccttgctgattatcggatgcagcaaaaccagagcggatttgatctgcgccatttcgttaacgtcaatttcaccctgccgaaagaaggcgagaaatatgttccgccagaggggcagtcactgcgcgaacatattgacggactttggccggtattaacgcgttctaccgaaaacaccgaaaaatgggattctctgttaccgctgccggaaccttatgtcgtgccgggcggacgctttcgcgaggtatattactgggacagttacttcaccatgttaggacttgccgaaagcggtcactgggataaagtcgcggatatggtggccaattttgctcatgaaatagacacttacggtcatattcccaacggcaaccgcagttactatttaagccgctcgcaaccgcccttctttgccctgatggtagagttactggcgcagcatgaaggcgatgccgcgttgaagcaatacctgccgcaaatgcaaaaagaatatgcttactggatggacggtgttgaaaacctgcaagccggacaacaggaaaaacgcgttgtcaaacttcaggatggtacccttctcaaccgctactgggacgatcgcgatacgccacgaccagagtcatgggtggaagatattgccaccgccaaaagcaatccgaatcgacctgccactgaaatttaccgcgacctgcgctctgccgctgcgtctggctgggatttcagctcgcgctggatggacaacccgcagcagttaaataccttacgcaccaccagcatcgtaccggtcgatctgaacagcctgatgtttaaaatggaaaaaatcctcgcccgcgccagcaaagctgccggagataacgcgatggcaaaccagtacgaaacgctggcaaatgcccgtcaaaaagggatcgaaaaatacctgtggaacgatcaacaaggctggtatgccgattacgacctgaaaagtcataaagtgcgcaatcagttaaccgcggccgccctgttcccgctgtacgtcaatgcggcagcgaaagatcgcgccaacaaaatggcgacggcgacgaaaacacatctgctgcaacccggcggcctgaacaccacgtcggtgaaaagtgggcaacaatgggatgcgccaaatggctgggcaccgttacagtgggtcgcgacagaaggattacaaaactacgggcaaaaagaggtggcgatggacattagctggcacttcctgaccaatgttcagcacacctatgaccgggagaaaaagctggtggaaaaatatgatgtcagcaccaccggaacggggggcggcggtggcgaatatccattacaggatggctttggctggaccaatggcgtgacgctgaaaatgctggatttgatctgcccgaaagagcaaccgtgtgacaatgttccggcgacgcgtccgaccgttaagtcagcaacgacgcaaccctcaaccaaagaggcacaacccacaccttaaccagcgcttactccgtctagacatcaccatcaccatcattaattaagtttgtgtttaaactgcaggcatgcaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaaaaaaaatccttacgtttcgctaaggatgtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgctcgatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaatt

SEQ ID NO: 131 - DNA, 27 bp, Artificial Sequence
  PCR primer
cgcaagttcttaagccaaaggagaatg

SEQ ID NO: 132 - DNA, 26 bp, Artificial Sequence
  PCR primer
aagcgctctagaaggtgtgggttgtg

SEQ ID NO: 133 - DNA, 6264 bp, Artificial Sequence
  plasmid pLybAL33 containing 6-His tagged treA
tagtcatgccccgcgcccaccggaaggagctgactgggttgaaggctctcaagggcatcggtcgataaatattctgaaatgagctgttgacaattaatcatcgaactagttaacttttacgcaagttcttaagccaaaggagaatgattgatgaaatcccccgcaccttctcgcccgcaaaaaatggcgttaattccagcctgtatctttttgtgtttcgctgcgctatcggtgcaggcagaagaaacaccggtaacaccacagccgcctgatattttattagggccgctgtttaatgatgtgcaaaacgccaaactttttccggaccaaaaaacctttgccgatgccgtgccgaacagcgatccgctgatgatccttgctgattatcggatgcagcaaaaccagagcggatttgatctgcgccatttcgttaacgtcaatttcaccctgccgaaagaaggcgagaaatatgttccgccagaggggcagtcactgcgcgaacatattgacggactttggccggtattaacgcgttctaccgaaaacaccgaaaaatgggattctctgttaccgctgccggaaccttatgtcgtgccgggcggacgctttcgcgaggtatattactgggacagttacttcaccatgttaggacttgccgaaagcggtcactgggataaagtcgcggatatggtggccaattttgctcatgaaatagacacttacggtcatattcccaacggcaaccgcagttactatttaagccgctcgcaaccgcccttctttgccctgatggtagagttactggcgcagcatgaaggcgatgccgcgttgaagcaatacctgccgcaaatgcaaaaagaatatgcttactggatggacggtgttgaaaacctgcaagccggacaacaggaaaaacgcgttgtcaaacttcaggatggtacccttctcaaccgctactgggacgatcgcgatacgccacgaccagagtcatgggtggaagatattgccaccgccaaaagcaatccgaatcgacctgccactgaaatttaccgcgacctgcgctctgccgctgcgtctggctgggatttcagctcgcgctggatggacaacccgcagcagttaaataccttacgcaccaccagcatcgtaccggtcgatctgaacagcctgatgtttaaaatggaaaaaatcctcgcccgcgccagcaaagctgccggagataacgcgatggcaaaccagtacgaaacgctggcaaatgcccgtcaaaaagggatcgaaaaatacctgtggaacgatcaacaaggctggtatgccgattacgacctgaaaagtcataaagtgcgcaatcagttaaccgcggccgccctgttcccgctgtacgtcaatgcggcagcgaaagatcgcgccaacaaaatggcgacggcgacgaaaacacatctgctgcaacccggcggcctgaacaccacgtcggtgaaaagtgggcaacaatgggatgcgccaaatggctgggcaccgttacagtgggtcgcgacagaaggattacaaaactacgggcaaaaagaggtggcgatggacattagctggcacttcctgaccaatgttcagcacacctatgaccgggagaaaaagctggtggaaaaatatgatgtcagcaccaccggaacggggggcggcggtggcgaatatccattacaggatggctttggctggaccaatggcgtgacgctgaaaatgctggatttgatctgcccgaaagagcaaccgtgtgacaatgttccggcgacgcgtccgaccgttaagtcagcaacgacgcaaccctcaaccaaagaggcacaacccacaccttctagacatcaccatcaccatcattaattaagtttgtgtttaaactgcaggcatgcaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaaaaaaaatccttacgtttcgctaaggatgtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgctcgatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaatt

SEQ ID NO: 134 - DNA, 1698 bp, Escherichia coli
atgaaatcccccgcaccttctcgcccgcaaaaaatggcgttaattccagcctgtatctttttgtgtttcgctgcgctatcggtgcaggcagaagaaacaccggtaacaccacagccgcctgatattttattagggccgctgtttaatgatgtgcaaaacgccaaactttttccggaccaaaaaacctttgccgatgccgtgccgaacagcgatccgctgatgatccttgctgattatcggatgcagcaaaaccagagcggatttgatctgcgccatttcgttaacgtcaatttcaccctgccgaaagaaggcgagaaatatgttccgccagaggggcagtcactgcgcgaacatattgacggactttggccggtattaacgcgttctaccgaaaacaccgaaaaatgggattctctgttaccgctgccggaaccttatgtcgtgccgggcggacgctttcgcgaggtatattactgggacagttacttcaccatgttaggacttgccgaaagcggtcactgggataaagtcgcggatatggtggccaattttgctcatgaaatagacacttacggtcatattcccaacggcaaccgcagttactatttaagccgctcgcaaccgcccttctttgccctgatggtagagttactggcgcagcatgaaggcgatgccgcgttgaagcaatacctgccgcaaatgcaaaaagaatatgcttactggatggacggtgttgaaaacctgcaagccggacaacaggaaaaacgcgttgtcaaacttcaggatggtacccttctcaaccgctactgggacgatcgcgatacgccacgaccagagtcatgggtggaagatattgccaccgccaaaagcaatccgaatcgacctgccactgaaatttaccgcgacctgcgctctgccgctgcgtctggctgggatttcagctcgcgctggatggacaacccgcagcagttaaataccttacgcaccaccagcatcgtaccggtcgatctgaacagcctgatgtttaaaatggaaaaaatcctcgcccgcgccagcaaagctgccggagataacgcgatggcaaaccagtacgaaacgctggcaaatgcccgtcaaaaagggatcgaaaaatacctgtggaacgatcaacaaggctggtatgccgattacgacctgaaaagtcataaagtgcgcaatcagttaaccgcggccgccctgttcccgctgtacgtcaatgcggcagcgaaagatcgcgccaacaaaatggcgacggcgacgaaaacacatctgctgcaacccggcggcctgaacaccacgtcggtgaaaagtgggcaacaatgggatgcgccaaatggctgggcaccgttacagtgggtcgcgacagaaggattacaaaactacgggcaaaaagaggtggcgatggacattagctggcacttcctgaccaatgttcagcacacctatgaccgggagaaaaagctggtggaaaaatatgatgtcagcaccaccggaacggggggcggcggtggcgaatatccattacaggatggctttggctggaccaatggcgtgacgctgaaaatgctggatttgatctgcccgaaagagcaaccgtgtgacaatgttccggcgacgcgtccgaccgttaagtcagcaacgacgcaaccctcaaccaaagaggcacaacccacaccttaa

SEQ ID NO: 135 - protein, 565 aa, Escherichia coli
MKSPAPSRPQKMALIPACIFLCFAALSVQAEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 136 - DNA, 1738 bp, Artificial Sequence
  PCR product
cgcaagttcttaagccaaaggagaatgattgatgaaatcccccgcaccttctcgcccgcaaaaaatggcgttaattccagcctgtatctttttgtgtttcgctgcgctatcggtgcaggcagaagaaacaccggtaacaccacagccgcctgatattttattagggccgctgtttaatgatgtgcaaaacgccaaactttttccggaccaaaaaacctttgccgatgccgtgccgaacagcgatccgctgatgatccttgctgattatcggatgcagcaaaaccagagcggatttgatctgcgccatttcgttaacgtcaatttcaccctgccgaaagaaggcgagaaatatgttccgccagaggggcagtcactgcgcgaacatattgacggactttggccggtattaacgcgttctaccgaaaacaccgaaaaatgggattctctgttaccgctgccggaaccttatgtcgtgccgggcggacgctttcgcgaggtatattactgggacagttacttcaccatgttaggacttgccgaaagcggtcactgggataaagtcgcggatatggtggccaattttgctcatgaaatagacacttacggtcatattcccaacggcaaccgcagttactatttaagccgctcgcaaccgcccttctttgccctgatggtagagttactggcgcagcatgaaggcgatgccgcgttgaagcaatacctgccgcaaatgcaaaaagaatatgcttactggatggacggtgttgaaaacctgcaagccggacaacaggaaaaacgcgttgtcaaacttcaggatggtacccttctcaaccgctactgggacgatcgcgatacgccacgaccagagtcatgggtggaagatattgccaccgccaaaagcaatccgaatcgacctgccactgaaatttaccgcgacctgcgctctgccgctgcgtctggctgggatttcagctcgcgctggatggacaacccgcagcagttaaataccttacgcaccaccagcatcgtaccggtcgatctgaacagcctgatgtttaaaatggaaaaaatcctcgcccgcgccagcaaagctgccggagataacgcgatggcaaaccagtacgaaacgctggcaaatgcccgtcaaaaagggatcgaaaaatacctgtggaacgatcaacaaggctggtatgccgattacgacctgaaaagtcataaagtgcgcaatcagttaaccgcggccgccctgttcccgctgtacgtcaatgcggcagcgaaagatcgcgccaacaaaatggcgacggcgacgaaaacacatctgctgcaacccggcggcctgaacaccacgtcggtgaaaagtgggcaacaatgggatgcgccaaatggctgggcaccgttacagtgggtcgcgacagaaggattacaaaactacgggcaaaaagaggtggcgatggacattagctggcacttcctgaccaatgttcagcacacctatgaccgggagaaaaagctggtggaaaaatatgatgtcagcaccaccggaacggggggcggcggtggcgaatatccattacaggatggctttggctggaccaatggcgtgacgctgaaaatgctggatttgatctgcccgaaagagcaaccgtgtgacaatgttccggcgacgcgtccgaccgttaagtcagcaacgacgcaaccctcaaccaaagaggcacaacccacaccttctagagcgctt

SEQ ID NO: 137 - protein, 573 aa, Artificial Sequence
  treA with 6-His tag
MKSPAPSRPQKMALIPACIFLCFAALSVQAEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPSRHHHHHH

SEQ ID NO: 138 - DNA, 1269 bp, Artificial Sequence
  sequence of partially deleted Synechococcus upp
gagctcggtacccggggatcccacggcagcattacggctcagaccttggtcatgccctcgacaacagatctctacttcaccccagaggattgtgaggccgaagcgcagttgattcctaaggcgcactattgcccaattccctcgatctggggtcaccgcgcgggcaaccccagccaaaatccgcaggatgaaagcttcattcggcaggccgttcaggctttgctcaacgctgaagcctagcgaattcagtcagcagatcaaggagtaccaaacaggcgatcgccagcatcccccagccccggcacgataaagcctttgtcgttcagctgctcatcaatgatggcgctgtaaatcgtcaacgccgggtaggcttgactgagtttttgtagcgctggcggggcagccacaattgaaagcacccgcacttgctcagcagagacaccgcgatcgcgcagcaaatcaagggtatagagcagcgagccacctgtcgccagcatcgggtcgagaaccagaacgcgactgttcacttcaagttgctctggcaggtgattgaggtagcagcgcggttcaagactgacttcatcccgctcgcgcagaatcggcacgatcgccaagggttgcgaaaaatcgacgaactccgctggggtttctgcaagaggagtttgcaccgccgctggaatcgttggtagccattcccgcacagcctcataggcgagccagcggcccagctctgcgatcgcggtgcgaaacagaggcgtcggcgtctggcgatcgcgggcaatgcccagccagtgccgaattaagggatggggcggcacgaagatacgcagttgaggagccatgccaatcagcagaagacagctcctgattttaacgttcagaccccaggggaagcggaacggtgcaggaaggcaagcgcttctgcttcgggcagtggtgggccatagaagaacccttgcacagcatcacaaccaatcgcttctaagaaggcggcttgctcgaggcgttctacgccttctgcgatcgtgcgaagtttcaagaccttggccattgcaacaatcgcctgcacgatcgcttgatcgtcatggtcgtgcggcagatcgcgaataaagctgcgatcaattttgagagcattgatgggcaaacgcttgaggtaaccaaggctggaataacccgtcccaaaatcatctaaagcgacttgaaatcccatcgatcgggcttcctggagccattgcagtgggatcctctagagtcgacctgcaggcatgc

(19) United States
(12) Patent Application Publication
(10) Pub. No.: US 2012/0301563 A1
(43) Pub. Date: Nov. 29, 2012

(54) TRANSGENIC PHOTOSYNTHETIC MICROORGANISMS AND PHOTOBIOREACTOR

(75) Inventors: John Aikens, La Grange Park, IL (US); Robert J. Turner, Aurora, IL (US)

(73) Assignee: PROTERRO, INC., Princeton, NJ (US)

(21) Appl. No.: 13/289,950

(22) Filed: Nov. 4, 2011

Related U.S. Application Data
(63) Continuation-in-part of application No. 12/348,887, filed on Jan. 5, 2009.
(60) Provisional application No. 61/085,797, filed on Aug. 1, 2008.
(60) Provisional application No. 61/018,798, filed on Jan. 3, 2008.
(60) Provisional application No. 61/410,329, filed on Nov. 4, 2010.
(60) Provisional application No. 61/434,207, filed on Jan. 19, 2011.

(51) Int. Cl. C12N 1/12; C12N 1/13; A61K 35/74; A61P 17/00; A23L 1/48; C11B 1/00; C08B 37/00; C10L 1/18; C10L 1/19; C12P 19/14; A61P 19/02

(52) U.S. Cl. 424/780; 435/257.1; 435/99; 435/257.2; 554/175; 536/123.1; 44/385; 44/403; 426/61

(57) ABSTRACT

Provided herein are methods of producing and processing a feedstock from a photosynthetic microorganism. Also provided are various transgenic photosynthetic microorganism and photobioreactors for use in the methods.

### CROSS-REFERENCE TO RELATED APPLICATIONS

This application is a Continuation in Part of U.S. Nonprovisional application Ser. No. 12/348,887, filed Jan. 5, 2009, which claims priority to U.S. Provisional Application No. 61/085,797, filed Aug. 1, 2008, and U.S. Provisional Application No. 61/018,798, filed Jan. 3, 2008. This application also claims priority to U.S. Provisional Application No. 61/410,329, filed Nov. 4, 2010, and U.S. Provisional Application No. 61/434,207, filed Jan. 19, 2011. Each of the above applications is incorporated herein by reference in its entirety for all purposes.

### INCORPORATION-BY-REFERENCE OF MATERIAL SUBMITTED IN COMPUTER READABLE FORM

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to transgenic microorganisms and methods and devices for their cultivation.

### BACKGROUND

To address the world's increasing energy requirements, efficient and environmentally sound alternatives to the use of fossil fuels are sought after. Biofuels are a wide range of fuels which are in some way derived from biomass. The term is understood to cover solid biomass, liquid fuels and various biogases. Biofuels are gaining increased public and scientific attention, driven by factors such as oil price spikes, the need for increased energy security, and concern over greenhouse gas emissions from fossil fuels.

Alternative fuels, such as ethanol or biodiesel, can be produced from plant biomass. For example, the key ingredient used to produce ethanol from current processes is termed fermentable sugar. Most often, fermentable sugar is in the form of sucrose, glucose, or high-fructose corn syrup. Plants currently grown to produce such biomass include corn, sugarcane, soybeans, canola, jatropha, and so forth. But much of the plant biomass used to produce fermentable sugar requires extensive energy-intensive pre-processing. Further, use of such plant biomass can lead to soil depletion, erosion, and diversion of the food supply.

Cyanobacteria (also known as blue-green algae, blue-green bacteria, and Cyanophyta) is a phylum of bacteria that obtain their energy through photosynthesis. The unicellular cyanobacterium Synechocystis sp. PCC6803 was the third prokaryote and first photosynthetic organism whose genome was completely sequenced. Synechocystis spp. PCC 6803 (ATCC 27184) and Synechococcus elongatus PCC 7942 (ATCC 33912) are relatively well-studied, have genetic tools available and the sequences of their genomes are known (see e.g., Koksharova, O. A. and Wolk, C. P. 2002. Appl Microbiol Biotechnol 58, 123-137; Ikeuchil, M. and Satoshi Tabata, S. 2001. Photosynthesis Research 70, 73-83; Golden, S. S., Brusslan, J. and Haselkorn, R. 1987. Methods in Enzymology 153, 215-231; Friedberg, D. 1988. Methods in Enzymology 167, 736-747; Kaneko, T. et al. 1996. DNA Research 3, 109-136).

Photosynthetic microorganisms that are commercial cultivated include Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Scenecoccus sp., Scenecosystis sp., and Tolypothrix.

### SUMMARY OF THE INVENTION

Provided herein are methods, compositions, and devices for cultivation of photosynthetic microorganisms, isolation of feedstock therefrom, and processing of feedstock.

One aspect provides a method for processing a feedstock including cultivating a photosynthetic microorganism, isolating a feedstock from the photosynthetic microorganism, and processing the feedstock. In some embodiments, the method includes cultivating the photosynthetic microorganism in a photobioreactor including a non-gelatinous, solid cultivation support suitable for providing nutrients and moisture to photosynthetic microorganisms on at least a portion of a surface thereof, wherein said portion of the surface has a topography that allows photosynthetic microorganisms to adhere thereto when said portion of the surface is oriented non-horizontally; and a physical barrier covering at least said portion of the surface of the cultivation support, wherein the physical barrier is configured so as to allow inoculation of said portion of the surface of the cultivation support, formation and maintenance of an environment suitable for the cultivation of such photosynthetic microorganisms, and harvesting of such cultivated photosynthetic microorganisms.

In some embodiments of the method, the photosynthetic microorganism is a transgenic photosynthetic microorganism comprising an artificial DNA construct comprising, as operably associated components in the 5′ to 3′ direction of transcription: a promoter functional in the photosynthetic microorganism cell; a polynucleotide comprising a first nucleotide sequence encoding a polypeptide having disaccharide phosphate synthase activity and a second nucleotide sequence encoding a polypeptide having disaccharide phosphate phosphatase activity or a polynucleotide comprising a nucleotide sequence encoding a polypeptide having disaccharide phosphate synthase activity and disaccharide phosphate phosphatase activity; and a transcriptional termination sequence; wherein the transgenic photosynthetic microorganism accumulates increased levels of the disaccharide compared to a photosynthetic microorganism not comprising the DNA construct.

In some embodiments, the feedstock includes a biomass and processing the feedstock comprises treating the biomass with one or more biomass degrading enzymes derived from a non-vascular photosynthetic organism for a sufficient amount of time to degrade at least a portion of said biomass to form a biofuel, wherein, optionally, said biomass degrading enzyme is a cellulolytic enzyme, a hemicellulolytic enzyme, or a ligninolytic enzyme.

In some embodiments, processing the feedstock includes contacting under catalytic cracking conditions the feedstock with a catalytic composition thereby making a fuel product.

In some embodiments, cultivating the photosynthetic microorganism includes contacting the photosynthetic microorganism with a flue gas; and isolating the feedstock comprises extracting a fuel product from the photosynthetic microorganism.

In some embodiments, the feedstock comprises a first fuel product; and cultivating the photosynthetic microorganism comprises contacting the photosynthetic microorganism with a source of inorganic carbons comprising Δ13C thereby incorporating carbons from the source of inorganic carbons into the first fuel product. In some embodiments, the method includes obtaining a measurement of a Δ13C distribution of the processed feedstock; comparing the Δ13C distribution of the processed feedstock to a reference Δ13C distribution; selling carbon credits to an entity if the Δ13C distribution of the processed feedstock is less than the reference Δ13C distribution, wherein the entity is an owner or user of the processed feedstock.

In some embodiments, the processed feedstock comprises a food composition comprising at least 0.1% w/w biomass of the photosynthetic microorganism and one or more other edible ingredients, wherein the biomass comprises at least 10% oil by dry weight.

In some embodiments, the feedstock comprises a triglyceride oil and processing the feedstock comprises subjecting the triglyceride oil to one or more chemical reactions to generate alkanes, whereby fuel is produced, and, optionally, wherein the one or more chemical reactions is a chemical reaction selected from the group consisting of transesterification, hydrogenation, hydrocracking, deoxygenation, isomerization, interesterification, hydroxylation, hydrolysis to yield free fatty acids, and saponification.

In some embodiments, the feedstock comprises a saccharide and processing the feedstock comprises providing the saccharide as a carbon source.

In some embodiments, the photosynthetic microorganism is cultivated in the presence of a fixed carbon source, wherein the photosynthetic microorganism contain an exogenous gene; the photosynthetic microorganism accumulate at least 10% of their dry cell weight as lipid; and the fixed carbon source is selected from the group consisting of sorghum and depolymerized cellulosic material; and isolating lipid components from the cultured microorganisms.

In some embodiments, the feedstock comprises a saponified microbial lipid and the processed feedstock comprises a soap comprising the saponified microbial lipid.

In some embodiments, the feedstock comprises lipids at least 5% of the photosynthetic microorganism dry weight and processing the feedstock comprises saponifying the lipid to obtain a soap.

In some embodiments, the processed feedstock is used to treat mammalian skin or stimulate collagen synthesis in mammalian skin, wherein treating comprises applying, injecting, or orally administering the processed feedstock to a mammalian subject.

In some embodiments, the processed feedstock comprises a polysaccharide and hyaluronic acid.

In some embodiments, the processed feedstock comprises a polysaccharide and the processed feedstock is used to lubricate the joint of a mammal by injecting the processed feedstock into a cavity containing synovial fluid of the mammal.

In some embodiments, the photosynthetic microorganism is cultivated on glycerol as a source of fixed carbon to produce the feedstock; and processing the feedstock comprises separating lipid from aqueous components of the feedstock and, optionally, deoxygenating the lipid, hydrogenating the lip, or hydrocracking the lipid.

In some embodiments, the photosynthetic microorganism is cultivated in the presence of a fixed carbon source, wherein the photosynthetic microorganisms accumulate at least 10% of their dry cell weight as lipid; and the fixed carbon source is selected from the group consisting of glycerol, depolymerized cellulosic material, sucrose, molasses, glucose, arabinose, galactose, xylose, fructose, arabinose, mannose, acetate, and any combination of the foregoing; isolating the feedstock comprises isolating lipid components from the photosynthetic microorganisms; and processing the feedstock comprises subjecting the lipid components to one or more chemical reactions to generate straight chain alkanes to form renewable diesel.

In some embodiments, the photosynthetic microorganisms are capable of heterotrophic growth; and cultivating the photosynthetic microorganisms comprises cultivating the photosynthetic microorganisms in a culture medium comprising a processed feedstock comprising a disaccharide.

In some embodiments, isolating the feedstock comprises recovering lipid from biomass produced by the photosynthetic microorganisms and processing the feedstock comprises subjecting the lipid to transesterification to produce fatty acid ester(s) and glycerol; and the processing the feedstock further comprises adding the glycerol to a microbial culture to form a biodiesel.

In some embodiments, processing the feedstock comprises disrupting the photosynthetic microorganisms to form a homogenate nutraceutical composition.

In some embodiments, the processed feedstock comprises a polysaccharide and a biologically acceptable carrier that is administered to a subject to lower serum cholesterol.

In some embodiments, the processed feedstock comprises a polysaccharide and the processed feedstock is packaged in a sexually transmitted disease prevention kit or composition further comprising a prophylactic device.

In some embodiments, isolating the feedstock comprises adding isopropanol to the cultivated photosynthetic microorganisms and separating precipitated exopolysaccharide.

In some embodiments, photosynthetic microorganisms are cultivated at a density of at least about 50 grams of dry biomass per liter equivalent. In some embodiments, the cultivation support comprises a flexible material. In some embodiments, the cultivation support comprises one or more rigid materials. In some embodiments, the cultivation support comprises at least two layers, a first layer adjacent to a second layer, the first layer comprising a high surface area growth material and the second layer comprises a permeable type material. In some embodiments, the cultivation support comprises flexibly connected rigid portions, wherein the rigid portions are comprised of the one or more rigid materials. In some embodiments, the physical barrier it is at least substantially impermeable to solid particulate and liquid but does not prevent the transport of gas or vapor to and from the space proximate to said portion of the surface of the cultivation support nor actinic irradiation of said portion of the surface of the cultivation support. In some embodiments, the photobioreactor comprises a source of actinic radiation situated to provide photosynthetically active radiation to at least a portion of the cultivation support. In some embodiments, the physical barrier is between the cultivation support and a source of actinic radiation and is sufficiently transparent to such actinic radiation and sufficiently gas permeable to allow for photosynthesis by the photosynthetic microorganisms during cultivation. In some embodiments, the physical barrier is sufficiently impermeable to water vapor so that the cultivation support upon being moistened will retain enough of the moisture so the photosynthetic microorganisms remain adequately hydrated during cultivation. In some embodiments, the physical barrier is configured to enclose the cultivation support and any photosynthetic microorganisms thereon, and to be releasably sealed during at least a portion of the cultivation of the photosynthetic microorganisms. In some embodiments, the photobioreactor comprises a single cultivation support. In some embodiments, the photobioreactor comprises a plurality of cultivation supports. In some embodiments, the physical barrier is flexible. In some embodiments, the physical barrier further comprises a first portion that is at least substantially impermeable to solid particulate, liquid, gas, and vapor, and a second portion that is permeable to gas and vapor but at least substantially impermeable to solid particulate and liquid. In some embodiments, the physical barrier further comprises a first portion that is at least substantially impermeable to solid particulate, liquid, gas, and vapor, and a second portion that is permeable to gas and vapor but at least substantially impermeable to solid particulate and liquid, the second portion of the barrier has a gas or vapor exchange rate that is from at least about 5 Gurley seconds to no greater than about 10,000 Gurley seconds. In some embodiments, the physical barrier further comprises a first portion that is at least substantially impermeable to solid particulate, liquid, gas, and vapor, and a second portion that is permeable to gas and vapor but at least substantially impermeable to solid particulate and liquid, the second portion of the barrier comprises a selective membrane comprising olefin fiber or polyethylene fiber material, polytetrafluoroethylene filtration media, cellulosic filter material, fiberglass filter material, polyester filter material, polyacrylate filter material, polysulfone membranes, or nylon membranes. In some embodiments, the physical barrier further comprises a first portion that is at least substantially impermeable to solid particulate, liquid, gas, and vapor, and a second portion that is permeable to gas and vapor but at least substantially impermeable to solid particulate and liquid, the first portion is at least substantially transparent to actinic radiation and the second portion is not at least substantially transparent to actinic radiation, and the configuration of the first and second portions relative to each other and at least said portion of the surface of the cultivation support is such that there a sufficient amount of actinic radiation and gas exchange to support photosynthesis by photosynthetic microorganisms. In some embodiments, the cultivation support comprises a fabric. In some embodiments, the cultivation support comprises a fabric, the fabric comprising fibers that are natural, modified natural, synthetic, or a combination thereof. In some embodiments, the cultivation support comprises a fabric, the fabric comprising a woven fabric, a knitted fabric, a felt, a mesh of cross-linked fiber polymers, or a combination thereof. In some embodiments, the cultivation support comprises a natural fiber selected from the group consisting of cotton, wool, hemp, tree fiber, other cellulosic fibers, and combinations thereof. In some embodiments, the cultivation support comprises a modified natural fiber selected from the group consisting of nitrocellulose, cellulose acetate, cellulose sulfonate, crosslinked starches, and combinations thereof, and a synthetic fiber selected from the group consisting of polyester, polyacrylate, polyamine, polyamide, polysulfone, and combinations thereof. In some embodiments, the cultivation support is coated with a moisture absorbent polymer. In some embodiments, the fabric, the fiber of the fabric, or both, are coated with a moisture absorbent polymer. In some embodiments, the moisture absorbent polymer is selected from the group consisting of agar, polyacrylate, polyamide, polyamine, polyethylene glycol, modified starches, and combinations thereof. In some embodiments, the photobioreactor comprises water, nutrients, or a combination thereof on, within, or on and within, the cultivation support. In some embodiments, the photobioreactor comprises one or more attachment points for attaching the photobioreactor to a structure. In some embodiments, the solid cultivation support further comprises one or more attachment points for attaching the cultivation support. In some embodiments, the photobioreactor comprises at least one of a fluid supply system, a nutrient supply system, a gas supply system, and a microorganism supply system. In some embodiments, at least a portion of the photosynthetic microorganisms are adhered to the solid cultivation support. In some embodiments, the photobioreactor comprises a plurality of solid cultivation supports radiating outward from a central point. In some embodiments, the photobioreactor comprises a conveyance system that moves a plurality of solid cultivation supports radiating outward from a central point so as to optimize position of one or more solid cultivation supports for receiving light. In some embodiments, the physical barrier comprises at least a portion sufficiently transparent to actinic radiation and the position of the portion is movable to optimize receipt of light by the solid cultivation support. In some embodiments, the solid cultivation support comprises a material having loops or is terry cloth.

In some embodiments, the photosynthetic microorganism comprises a polynucleotide comprising a nucleotide sequence encoding a polypeptide selected from the group consisting of: SEQ ID NO: 2 or a sequence 95% identical thereto having sucrose phosphate synthase and sucrose phosphate phosphatase (ASF) activity; SEQ ID NO: 4 or a sequence 95% identical thereto having sucrose phosphate synthase (SPS) activity; SEQ ID NO: 6 or a sequence 95% identical thereto having a sucrose phosphate phosphatase (SPP) activity; SEQ ID NO: 77 or a sequence 95% identical thereto having trehalose phosphate synthase (TPS) activity; SEQ ID NO: 79 or a sequence 95% identical thereto having trehalose phosphate phosphatase (TPP) activity; SEQ ID NO: 81 or a sequence 95% identical thereto having glucosylglycerol phosphate synthase (GPS) activity; SEQ ID NO: 83 or a sequence 95% identical thereto having glucosylglycerol phosphate phosphatase (GPP) activity; SEQ ID NO: 85 or a sequence 95% identical thereto having mannosylfructose phosphate synthase (MPS) activity; and SEQ ID NO: 87 or a sequence 95% identical thereto having mannosylfructose phosphate phosphatase (MPP) activity

In some embodiments, the photosynthetic microorganism comprises an isolated polynucleotide comprising SEQ ID NO: 1 or a sequence 95% identical thereto encoding sucrose phosphate synthase/sucrose phosphate phosphatase (ASF) activity; SEQ ID NO: 3 or a sequence 95% identical thereto encoding sucrose phosphate synthase (SPS) activity; SEQ ID NO: 5 or a sequence 95% identical thereto encoding sucrose phosphate phosphatase (SPP) activity; SEQ ID NO: 76 or a sequence 95% identical thereto encoding trehalose phosphate synthase (TPS) activity; SEQ ID NO: 78 or a sequence 95% identical thereto encoding trehalose phosphate phosphatase (TPP) activity; SEQ ID NO: 80 or a sequence 95% identical thereto encoding glucosylglycerol phosphate synthase (GPS) activity; SEQ ID NO: 82 or a sequence 95% identical thereto encoding glucosylglycerol phosphate phosphatase (GPP) activity; SEQ ID NO: 84 or a sequence 95% identical thereto encoding mannosylfructose phosphate synthase (MPS) activity; and SEQ ID NO: 86 or a sequence 95% identical thereto encoding mannosylfructose phosphate phosphatase (MPP) activity.

In some embodiments, the photosynthetic microorganism comprises an isolated polynucleotide that hybridizes under stringent conditions to a nucleic acid sequence selected from the group consisting of SEQ ID NO: 1, wherein the isolated polynucleotide encodes a polypeptide having ASF activity; SEQ ID NO: 3, wherein the isolated polynucleotide encodes a polypeptide having SPS activity; SEQ ID NO: 5, wherein the isolated polynucleotide encodes a polypeptide having SPP activity; SEQ ID NO: 76, wherein the isolated polynucleotide encodes a polypeptide having TPS activity; SEQ ID NO: 78, wherein the isolated polynucleotide encodes a polypeptide having TPP activity; SEQ ID NO: 80, wherein the isolated polynucleotide encodes a polypeptide having GPS activity; SEQ ID NO: 82, wherein the isolated polynucleotide encodes a polypeptide having GPP activity; SEQ ID NO: 84, wherein the isolated polynucleotide encodes a polypeptide having MPS activity; SEQ ID NO: 86, wherein the isolated polynucleotide encodes a polypeptide having MPP activity; wherein said stringent conditions comprise incubation at 65° C. in a solution comprising 6×SSC (0.9 M sodium chloride and 0.09 M sodium citrate).

In some embodiments, the photosynthetic microorganism comprises an isolated polynucleotide complementary to any of the above polynucleotides.

In some embodiments, monomers of the accumulated disaccharide are endogenous to the photosynthetic microorganism. In some embodiments, the photosynthetic microorganism comprises a cyanobacterium cell, a photosynthetic bacteria; or a green algae. In some embodiments, the photosynthetic microorganism comprises a cyanobacterium cell. In some embodiments, the photosynthetic microorganism comprises a Synechococcus or a Synechocystis. In some embodiments, the promoter is an inducible promoter. In some embodiments, the promoter is selected from the group consisting of carB, nirA, psbAII, dnaK, kaiA, and λPR. In some embodiments, the DNA construct comprises a nucleotide sequence selected from the group consisting of SEQ ID NO: 19 (pLybAL11 encoding asf); SEQ ID NO: 20 (pLybAL12 encoding asf); SEQ ID NO: 44 (pLybAL15 encoding asf); SEQ ID NO: 45 (pLybAL16 encoding asf); SEQ ID NO: 46 (pLybAL17 encoding asf); SEQ ID NO: 47 (pLybAL18 encoding asf); SEQ ID NO: 48 (pLybAL19 encoding asf); SEQ ID NO: 49 (pLybAL21 encoding asf); SEQ ID NO: 50 (pLybAL22 encoding asf); SEQ ID NO: 51 (pLybAL13f encoding asf); SEQ ID NO: 52 (pLyAL13r encoding asf); SEQ ID NO: 53 (pLybAL14f encoding asf); SEQ ID NO: 54 (pLybAL14r encoding asf); SEQ ID NO: 65 (pLybAL7f encoding asf); SEQ ID NO: 69 (pLybAL8f encoding asf); SEQ ID NO: 118 (pLybAL23 encoding tps and tpp); SEQ ID NO: 121 (pLybAL28 encoding tps and tpp); SEQ ID NO: 122 (pLybAL29 encoding tps and tpp); SEQ ID NO: 123 (pLybAL30 encoding tps and tpp); SEQ ID NO: 124 (pLybAL31 encoding tps and tpp); SEQ ID NO: 125 (pLybAL36 encoding tps and tpp); SEQ ID NO: 126 (pLybAL37 encoding tps and tpp); SEQ ID NO: 130 (pLybAL24 encoding tps and tpp); and SEQ ID NO: 133 (pLybAL33 encoding tps and tpp). In some embodiments, the photosynthetic microorganism accumulates at least about 0.1 micrograms of the disaccharide per minute per gram dry biomass. In some embodiments, the photosynthetic microorganism accumulates at least about 0.1 micrograms of the disaccharide per minute per gram dry biomass up to about 10 micrograms of the disaccharide per minute per gram dry biomass. In some embodiments, the photosynthetic microorganism does not comprise a nucleotide sequence selected from the group consisting of SEQ ID NO: 70, SEQ ID NO: 72, and SEQ ID NO: 74, or a nucleotide variant thereof having at least 95% identity thereto and invertase activity or sucraseferridoxin activity. In some embodiments, the photosynthetic microorganism does not express a polypeptide sequence selected from the group consisting of SEQ ID NO: 71, SEQ ID NO: 73, and SEQ ID NO: 75, or a polypeptide variant thereof having at least 95% identity thereto and invertase activity or sucraseferridoxin activity. In some embodiments, the photosynthetic microorganism expresses a small interfering RNA specific a nucleotide sequence selected from the group consisting of SEQ ID NO: 70, SEQ ID NO: 72, and SEQ ID NO: 74, or a nucleotide variant thereof having at least 95% identity thereto and invertase activity or sucraseferridoxin activity. In some embodiments, the photosynthetic microorganism comprises an isolated polynucleotide comprising SEQ ID NO: 94 or a sequence 95% identical thereto encoding an active porin polypeptide; an isolated polynucleotide encoding a polypeptide comprising SEQ ID NO: 95 or a sequence 95% identical thereto and having porin activity; or an isolated polynucleotide comprising SEQ ID NO: 91 (pLybAL32 encoding a porin); wherein the accumulated disaccacharide is sucrose, the cell expresses porin, and the expressed porin secretes the accumulated sucrose from the cell.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a front view of the photobioreactor of the invention including a solid cultivation support, an outer protective transparent barrier layer, a selective panel, resealable closures, and support elements for suspending the device.

FIG. 2 illustrates a side view of the photobioreactor of the invention including a solid cultivation support, an outer protective transparent barrier layer, a selective panel, resealable closures, and support elements for suspending the device.

FIG. 3 illustrates an arrangement of multiple photobioreactors or cultivation supports of the invention along multiple closed loop conveyor systems radiating out from common inoculation and harvesting centers to comprise a photobioreactor farm.

FIG. 4 is a cartoon depicting photosynthetic production of sucrose in cyanobacteria.

FIG. 5 is a polypeptide sequence alignment of the Synechocystis spp. PCC 6803 (Ssp6803) sucrose phosphate synthase (SPS) and sucrose phosphate phosphatase (SPP) proteins with the Synechococcus elongatus PCC 7942 (Selo7942) active SPS/SPP fusion (ASF). Ssp6803 contains separate genes encoding SPS and SPP activities. The SPS protein from Synechocystis spp. PCC 6803 bears a presumably inactive SPP domain, as many of the active site residues are not conserved. The canonical HAD hydrolase active site residues are shown above the alignment with conserved amino acids shown underlined and non-conserved residues double underlined. An eight amino acid insertion within the inactive SPP domain of Synechocystis spp. PCC 6803 SPS is italicized. Further details regarding methodology are provided in Example 4.

FIG. 6 is schematic depiction of pLybAL11. pLybAL11 allows construction of libraries of cyanobacterial DNA and selection for promoter sequences. The promoterless asf gene is behind bidirectional terminators, separated by a multiple cloning site (MCS). oriV allows for plasmid replication in most Gram-negative organisms. oriT allows for conjugal transfer of the plasmid from E. coli to a chosen cyanobacterium (or other organism) with the assistance of the pRK2013 helper plasmid. The β-lactamase gene (bla) is present for selection in E. coli. DNA libraries can be constructed in E. coli by cloning cyanobacterial genomic DNA into the MCS. The plasmid library can then be transferred to cyanobacteria by conjugation or direct transformation. Active promoters can then be isolated by selection for resistance to chloramphenicol through expression of the chloramphenicol acetyltransferase gene (cat). The strength of the promoters can be assessed by both assay for chloramphenicol acetyltransferase activity and direct examination of sucrose production. Further details regarding methodology are provided in Example 5.

FIG. 7 is schematic depiction of pLybAL12. pLybAL12 allows analysis of the capacity of preselected promoters to drive asf expression. The only difference between pLybAL12 and pLybAL11 is the presence of an active promoter in front of the chloramphenicol acetyltransferase gene (cat). Specific DNA sequences isolated from cyanobacterial chromosomal DNA amplified by PCR can be cloned into the MCS. Both chloramphenicol and ampicillin can be used for selection in E. coli. The plasmid library can then be transferred to cyanobacteria by conjugation or direct transformation. Plasmid bearing cyanobacteria can then be isolated by selection for resistance to chloramphenicol through expression of the chloramphenicol acetyltransferase gene (cat). The strength of the promoters can be assessed by both assay for chloramphenicol acetyltransferase activity and direct examination of sucrose production. Further details regarding methodology are provided in Example 5.

FIG. 8 is a cartoon depicting construction of a cyanobacterial promoter library. Further details regarding methodology are provided in Example 8.

FIG. 9 is a schematic diagram depicting pSMART-LCKan. Further details regarding methodology are provided in Example 8.

FIG. 10 is a sequence listing showing a possible promoter within Synechococcus elongatus PCC 7942 asf. Shown is the amplified PCR product containing the asf gene from Synechococcus elongatus PCC 7942 that was cloned upstream of the chloramphenicol resistance marker. The regions of asf encoding the sucrose phosphate synthase and sucrose phosphate phosphatase polypeptide activities are single underlined and double underlined, respectively. All DNA sequence elements are italicized and labeled above. Start and Stop represent the start and stop codons, respectively. SD represents the Shine-Delgarno sequence. The −35 and −10 regions of the putative promoters are highlighted in gray. Further details regarding methodology are provided in Example 8.

FIG. 11 is a schematic diagram depicting a two-step protocol for markerless deletion of genes in the cyanobacterial genome. This strategy assumes that the cyanobacterial strain being used has had its upp gene deleted. The upp gene will have been deleted during the sucrose biosynthetic insertions. The gene of interest that has been targeted for deletion must be identified. The starting strain is resistant to 5-fluorouracil, but sensitive to kanamycin. The gene is either completely or partially deleted by the insertion of a cassette containing a kanamycin resistance marker and an active upp, making the strain resistant to kanamycin, but sensitive to 5-fluorouracil. The upp and kanamycin resistance markers can then be removed, making the strain once again resistant to 5-fluorouracil, but sensitive to kanamycin. Further details regarding methodology are provided in Example 12.

FIG. 12 is a schematic diagram of a photobioreactor embodiment. FIG. 12A provides a front view while FIG. 12B provides a side view. The photobioreactor includes suspension element (6); culture media supply (8); gas supply (10); growth surface (2); outer barrier layer (7); quick connector; and product harvest line (9).

FIG. 13 is a schematic diagram of a growth surface in a single material format (FIG. 13A) and a hybrid material format (FIG. 13B).

### DETAILED DESCRIPTION OF THE INVENTION

The present application relates to fermentable sugar accumulating photosynthetic microorganisms, solid-phase photobioreactor devices, and methods of using each. Described herein is technology directed to transgenic photosynthetic microorganisms engineered to produce disaccharides (e.g., sucrose, trehalose, glucosylglycerol, or mannosylfructose), thereby providing a fermentation-ready sugar feedstock (e.g., Protose™ sugar). Exemplary organisms include transgenic cyanobacteria (e.g., Synechococcus or Synechocystis) engineered to have sucrose phosphate synthase (sps) enzymatic activity and sucrose phosphate phosphatase (spp) enzymatic activity, through expression of two polypeptides (SPS, SPP) or one fusion polypeptide (ASF) having synthase activity and phosphatase activity. Embodiments described herein can be effectively combined with a bioreactor chamber having solid cultivation support, optimized for growth of photosynthetic microorganisms, especially those having a filamentous, sheet, or ball morphology.

Subject matter pertinent to transgenic photosynthetic microorganisms, optimized bioreactors, and sugar produced and derived from non-vascular photosynthetic organisms include US Patent Application Publication No. 2009/0181434 and PCT Publication WO 2009/089185, each of which is incorporated herein by reference in its entirety. Except as otherwise noted, compositions and processes of the present disclosure can be carried out in accordance with those disclosed in these publications.

Feedstock

One aspect provides methods for using the feedstock comprising biomass and/or useful compounds from photosynthetic microorganisms and/or bioreactors described herein and in applications incorporated by reference herein.

Because of the greater efficiency and lower environmental impact of growing photosynthetic microorganisms (especially in optimized bioreactors) compared to higher plants, technology described herein represents important improvements in sustainability over current biofuel production practices. Such technology can provide biomass and/or useful compounds (e.g., Protose™ sugar, lipids, fatty acid esters, fatty acids, aldehydes, alcohols, or alkanes) for food sources or alternative fuels such as ethanol or biodiesel. Relative to plants currently grown to produce biomass such as corn, sugarcane, soybeans, canola, or jatropha, photosynthetic microorganisms, such as cyanobacteria, produce biomass at a much faster rate, leading to much greater productivity. In addition, direct production of disaccharides by microorganisms avoids much of the extensive energy-intensive pre-processing of using plant biomass to produce fermentable sugar. Further, the use of phototrophic microorganisms instead of plants can lead to higher yields of fermentable sugars without soil depletion, erosion, and diversion of the food supply. Relative to other microorganisms, preference is given to phototrophic microorganisms because their sources of carbon (CO₂) and energy (light) can be supplied from the environment, making them far less expensive to cultivate. In addition, phototrophic microorganisms can be utilized to consume carbon emissions from industrial processes, thus providing further benefits to the environment. For example, the phototrophic microorganisms, such as cyanobacteria can be grown in a bioreactor located adjacent to an industrial plant producing CO₂ emissions, such as a fermentation plant or power plant. These CO₂ emissions may be used as a feed to the bioreactor to supply or supplement the necessary CO₂ for growth of the cyanobacteria. The bioreactor can be located adjacent or near any CO₂ producing plant or factory.

Sugar

A feedstock from a photosynthetic microorganism can be a fermentable sugar feedstock. A fermentable sugar can be a fermentable disaccharide sugar. Examples of fermentable disaccharide sugars include, but are not limited to sucrose and trehalose. The fermentable sugar can be a disaccharide not generally utilized by photosynthetic microorganisms. For example, trehalose is not generally utilized by cyanobacteria and therefore can accumulate within the cultivated biomass without substantial degradation by endogenous metabolic pathways. The fermentable sugar can be a disaccharide that is generally utilized by photosynthetic microorganisms. For a disaccharide not used as a primary energy source, the disaccharide can often be accumulated to sufficient levels even in the presence of endogenous metabolic pathways. Where endogenous degradation pathways specific for the target fermentable sugar, the photosynthetic microorganism can be engineered to reduce or eliminate such activity. For example, a cyanobacterium engineered to accumulate sucrose can be further engineered to reduce or eliminate sucrose invertase activity. In various embodiments, feedstock can be produced from photosynthetic microorganisms that synthesize fermentable disaccharide sugar in response to osmotic or matric water stress. In other embodiments, feedstock can be produced from transgenic strains of photosynthetic microorganisms engineered to accumulate fermentable disaccharide sugar in the absence of, or reduced levels of, osmotic stress. Advantageously, the foregoing methods of synthesizing fermentable disaccharide sugar can be adapted to occur within photobioreactors described herein.

In the fermentable sugar accumulating photosynthetic microorganisms, it may be preferable to produce a dissaccharide sugar not generally utilized by the photosynthetic microorganisms, which therefore can accumulate within the cultivated biomass (e.g., sucrose, trehalose). In some embodiments, photosynthetic microorganisms are genetically engineered to synthesize a dissaccharide sugar normally produced according to osmotic stress pathways (e.g., sucrose or trehalose) such that the sugar is produced in the absence of, or at reduced levels of, osmotic stress. Because of the greater efficiency and lower environmental impact of growing photosynthetic microorganisms compared to higher plants, the method represents important improvements in sustainability over current biofuel production practices. Advantageously, the foregoing method of synthesizing a dissaccharide sugar has been adapted to occur within the photobioreactor(s) of the present invention.

Uses of a sugar, for example a feedstock sugar, produced in a nonvascular photosynthetic organism include both biological and chemical processes. For processes described generally herein, a sugar derived from a nonvascular photosynthetic organism can be used directly as received, concentrated to remove water, or purified by commonly understood methods.

Feedstock Use

A sugar derived from a nonvascular photosynthetic organism (e.g., Protose™ sugar) can provide a basic carbon source for the cultivation of microorganisms. Such microorganism may be of prokaryotic or eukaryotic nature or may use the sugar in metabolic function and growth. For example, sugar derived from a nonvascular photosynthetic organism can provide feedstock for growth of yeast and corresponding fermentation production of ethanol. As another example, sugar derived from a nonvascular photosynthetic organism can provide feedstock for growth of photosynthetic or non-photosynthetic microorganisms or algae in a fermentation tank (e.g., feeding the microbes sugars rather than sunlight). Within the constraints of fermentative cultivation, it is understood that the processes may include submerged liquid, solid state, aerobic, anaerobic, and include any number of environmental conditions generally utilized for the organism of choice.

A sugar derived from a nonvascular photosynthetic organism (e.g., Protose™ sugar) can provide a basic carbon source for the cultivation of microorganisms. Such microorganism may be of prokaryotic or eukaryotic nature or may use the sugar in metabolic function and growth. For example, sugar derived from a nonvascular photosynthetic organism can provide feedstock for growth of yeast and corresponding fermentation production of ethanol. As another example, sugar derived from a nonvascular photosynthetic organism can provide feedstock for growth of photosynthetic or non-photosynthetic microorganisms or algae in a fermentation tank (e.g., feeding the microbes sugars rather than sunlight). Within the constraints of fermentative cultivation, it is understood that the processes may include submerged liquid, solid state, aerobic, anaerobic, and include any number of environmental conditions generally utilized for the organism of choice.

A sugar derived from a nonvascular photosynthetic organism (e.g., Protose™ sugar) can also be used as a feedstock under circumstances in which prokaryotic or eukaryotic cells are not actively growing but rather in a stationary phase of their life cycle. Commonly known as a whole cell biocatalyst, the biological machinery found within the resting cells is capable of converting organic molecular feedstock materials to products without undergoing cellular division. Under these conditions, the cells may be circulated freely in a solution or immobilized.

A sugar derived from a nonvascular photosynthetic organism (e.g., Protose™ sugar) can find application in enzyme catalyzed reactions whereby an isolated mixture or substantially purified enzyme can be made to react with the sugar moiety in such a manner that the sugar species is converted to a product. Similarly, biomass from a nonvascular photosynthetic organism can be reacted with a biomass degrading enzyme, such as a cellulolytic, hemicellulolytic or ligninolytic enzyme. The enzyme catalyst can be prepared in any number of forms including dissolved or dispersed in a solvent of choice or immobilized onto a substrate. Further, the enzyme can function in a number of environments including liquid phase, multiple liquid phases, solid phase or gaseous phases.

A sugar derived from a nonvascular photosynthetic organism (e.g., Protose™ sugar) can be useful as a feedstock material for conventional chemical processes in which an organic material such as sugar is chemically transformed into a different species. This transformed chemical material can either be a final product or an intermediate that can be subjected to additional chemical or biological mediated transformations. Chemical transformation processes can include pyrolysis, hydrolysis, reduction, oxidation, rearrangement, reforming, or polymerization either in single or multiple steps as determined by the desired product under commonly used conditions.

Furthermore, chemical products derived from processes using technology described herein, such as sugar (e.g., Protose™ sugar) or derived from a nonvascular photosynthetic organism can be used as fine chemicals, commodity chemicals, food additives, nutraceuticals, pharmaceuticals or fuels. For example, lipid components can be isolated from a nonvascular photosynthetic organism and subjected to chemical reactions to generate compounds, such as alkanes or cracked alkanes, useful for renewable fuels or fuel additives.

Except as otherwise noted, compositions and processes of the present disclosure can be carried out in accordance with those disclosed in, for example, US Patent Application Publication Nos. 2010/0050301, 2009/0280545, 2009/0269816, 2009/0253169, 2009/0246766, 2009/0126260, 2009/0123977, 2009/0087890, 2010/0239712, 2010/0170144, 2010/0151567, 2010/0151539, 2010/0151538, 2010/0151535, 2010/0151112, 2009/0305942, 2009/0285850, 2009/0274736, 2009/0148918, 2009/0069213, 2009/0061493, 2009/0047721, 2009/0035842, 2009/0023180, 2009/0011480, 2009/0004715, 2008/0299147, 2008/0124756, 2007/0191303, 2007/0167398, 2007/0167397, 2007/0167396, 2007/0166797, 2007/0166449, and 2007/0166266, each of which is incorporated herein by reference in its entirety.

Photosynthetic Microorganism

Provided herein is a photosynthetic microorganism genetically engineered to accumulate a dissaccharide sugar. The photosynthetic microorganism can be, for example, a naturally photosynthetic microorganism, such as a cyanobacterium, or an engineered photosynthetic microorganism, such as an artificially photosynthetic bacterium. Examples of the accumulated dissaccharide sugar include, but are not limited to sucrose, trehalose, gluocosylglycerol, and mannosylfructose. In various embodiments, one or more genes encoding the protein(s) responsible for producing the desired dissaccharide from corresponding phosphorylated monomers is engineered in a host photosynthetic microorganism (e.g., cyanobacterium) so as to result in the accumulation of the desired dissaccharide. In some embodiments, an endogenous pathway of the host photosynthetic microorganism is engineered so as to accumulate a dissaccharide sugar. For example, the osmotic sucrose pathway in cyanobacteria can be engineered to accumulate sucrose in the absence of osmotic stress. In some embodiments, an exogenous dissaccharide pathway is engineered in cyanobacteria so as to accumulate a dissaccharide sugar. For example, the osmotic trehalose pathway from E. coli can be engineered to accumulate trehalose in cyanobacteria.

Synthase and Phosphotase

A photosynthetic microorganism can be transformed so as to have a synthase activity and a phosphotase activity for the desired dissaccharide. For example, a cyanobacterium can be engineered to have sucrose phosphate synthase activity and sucrose phosphate phosphatase activity. As another example, a cyanobacterium can be engineered to have trehalose phosphate synthase activity and trehalose phosphate phosphatase activity. As another example, a cyanobacterium can be engineered to have gluocosylglycerol phosphate synthase activity and gluocosylglycerol phosphate phosphatase activity. As another example, a cyanobacterium can be engineered to have mannosylfructose phosphate synthase activity and mannosylfructose phosphate phosphatase activity. It is contemplated these activities can likewise be engineered in other photosynthetic microorganisms.

Synthase activity and phosphotase activity can be engineered into a photosynthetic microorganism by way of the individual genes, one encoding a polypeptide having synthase activity and the other encoding a polypeptide having phosphatase activity; or by one gene encoding both synthase activity and phosphatase activity. For example, synthase activity and phosphatase activity can be present in a fusion polypeptide.

The monomeric sugars of the desired dissaccharide can be endogenous or exogenous to the photosynthetic microorganism. Where monomeric sugars of the desired dissaccharide are endogenous, the photosynthetic microorganism can be engineered to produce increased levels of such monomers. Where monomeric sugars of the desired dissaccharide are exogenous, the photosynthetic microorganism can be engineered to produce such exogenous monomers.

The photosynthetic microorganism can be engineered to synthesize and accumulate the desired dissaccharide continuously, after some developmental state, or upon being induced to do so. Induction of dissaccharide synthesis can be according to the actions of an inducible promoter associated with the encoded synthase or phosphotase and an inducing agent, as discussed in further detail herein.

In some embodiments, transformed cyanobacteria, as described herein, can accumulate at least about 0.1 micrograms of a dissaccharide (e.g., sucrose, trehalose, glucosylglycerol, or mannosylfructose) per minute per gram dry biomass. In some embodiments, transformed cyanobacteria can accumulate at least about 0.1 up to about 10 micrograms of a dissaccharide (e.g., sucrose, trehalose, glucosylglycerol, or mannosylfructose) per minute per gram dry biomass. For example, transformed cyanobacteria can accumulate at least about 0.2, at least about 0.3, at least about 0.4, at least about 0.5, at least about 0.6, at least about 0.7, at least about 0.8, or at least about 0.9 micrograms of a dissaccharide (e.g., sucrose, trehalose, glucosylglycerol, or mannosylfructose) per minute per gram dry biomass. In other embodiments, various transformed photosynthetic microorganisms accumulate similar amounts of a dissaccharide.

It is contemplated that that various embodiments will accumulate a disaccharide (e.g., sucrose, trehalose, glucosylglycerol, or mannosylfructose) at defined ranges of the values above. For example, some transformed cyanobacteria can accumulate at least about 0.1 up to about 0.9 micrograms of a disaccharide (e.g., sucrose, trehalose, glucosylglycerol, or mannosylfructose) per minute per gram dry biomass; at least about 0.1 up to about 0.8 micrograms of a disaccharide (e.g., sucrose, trehalose, glucosylglycerol, or mannosylfructose) per minute per gram dry biomass; at least about 0.1 up to about 0.7 micrograms of a disaccharide (e.g., sucrose, trehalose, glucosylglycerol, or mannosylfructose) per minute per gram dry biomass; etc. Similarly, some transformed cyanobacteria can accumulate at least about 0.2 up to about 1.0 micrograms of a disaccharide (e.g., sucrose, trehalose, glucosylglycerol, or mannosylfructose) per minute per gram dry biomass; at least about 0.3 up to about 1.0 micrograms of a disaccharide (e.g., sucrose, trehalose, glucosylglycerol, or mannosylfructose) per minute per gram dry biomass; at least about 0.4 up to about 1.0 micrograms of a disaccharide (e.g., sucrose, trehalose, glucosylglycerol, or mannosylfructose) per minute per gram dry biomass; at least about 0.5 up to about 1.0 micrograms of a disaccharide (e.g., sucrose, trehalose, glucosylglycerol, or mannosylfructose) per minute per gram dry biomass; at least about 0.6 up to about 1.0 micrograms of a disaccharide (e.g., sucrose, trehalose, glucosylglycerol, or mannosylfructose) per minute per gram dry biomass; at least about 0.7 up to about 1.0 micrograms of a disaccharide (e.g., sucrose, trehalose, glucosylglycerol, or mannosylfructose) per minute per gram dry biomass; at least about 0.8 up to about 1.0 micrograms of a disaccharide (e.g., sucrose, trehalose, glucosylglycerol, or mannosylfructose) per minute per gram dry biomass; or at least about 0.9 up to about 1.0 micrograms of a disaccharide (e.g., sucrose, trehalose, glucosylglycerol, or mannosylfructose) per minute per gram dry biomass. Methods for assaying sugar accumulation is host cells are well-known to those of skill in the art (see e.g., Example 10).

Host

The host genetically engineered to accumulate a dissaccharide sugar can be any photosynthetic microorganism. The photosynthetic microorganism can be, for example, a naturally photosynthetic microorganism, such as a cyanobacterium, or an engineered photosynthetic microorganism, such as an artificially photosynthetic bacterium. Exemplary microorganisms that are either naturally photosynthetic or can be engineered to be photosynthetic include, but are not limited to, bacteria; fungi; archaea; protists; microscopic plants, such as a green algae; and animals such as plankton, planarian, and amoeba. Examples of naturally occurring photosynthetic microorganisms include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechoccus sp., Synechocystis sp., and/or Tolypothrix.

Preferably, the host photosynthetic microorganism is a cyanobacterium. Cyanobacteria, also known as blue-green algae, are a broad range of oxygengenic photoautotophs. The host cyanobacterium can be any photosynthetic microorganism from the phylum Cyanophyta. The host cyanobacterium can have a unicellular or colonial (e.g., filaments, sheets, or balls) morphology. Preferably, the host cyanobacterium is a unicellular cyanobacterium. Examples of cyanobacteria that can be engineered to accumulate a disaccharide sugar include, but are not limited to, the genus Synechocystis, Synechococcus, Thermosynechococcus, Nostoc, Prochlorococcu, Microcystis, Anabaena, Spirulina, and Gloeobacter. Preferably the host cyanobacterium is a Synechocystis spp. or Synechococcus spp. More preferably, the host cyanobacterium is Synechococcus elongatus PCC 7942 (ATCC 33912) and/or Synechocystis spp. PCC 6803 (ATCC 27184).

Sucrose

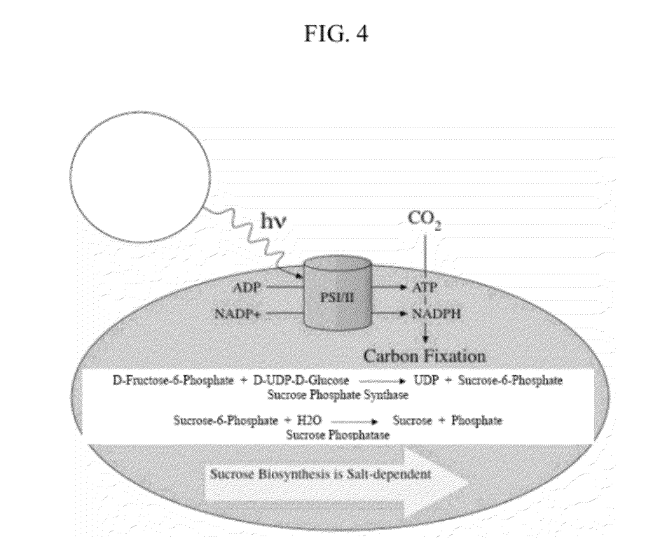

Biosynthesis of sucrose in a photosynthetic microorganism, such as cyanobacteria, can be accomplished through the catalytic action of two enzyme activities, sucrose phosphate synthase (sps) and sucrose phosphate phosphatase (spp), functioning in sequence (see e.g., FIG. 4). Such activities are present in some cyanobacteria for acclimation to osmotic and matric water stress (see e.g., Lunn, J. E. 2002. Plant Physiol 128, 1490-1500). Either or both of these activities can be engineered in a cyanobacterium so as to result in accumulation of sucrose.

A gene of particular interest for engineering a photosynthetic microorganism to accumulate sucrose is the active sps/spp fusion (asf) gene from Synechococcus elongatus PCC 7942. Asf has both sps and spp biosynthetic functions (see e.g., Example 4). In some embodiments, an ASF-encoding nucleotide sequence is cloned from its native source (e.g., Synechococcus elongatus PCC 7942) and inserted into a host cyanobacterium (see e.g., Examples 4-9). In some embodiments, a transformed host photosynthetic microorganism comprises an asf polynucleotide of SEQ ID NO: 1. In some embodiments, a photosynthetic microorganism is transformed with a nucleotide sequence encoding ASF polypeptide of SEQ ID NO: 2. In further embodiments, a transformed host photosynthetic microorganism comprises a nucleotide sequence having at least about 80% sequence identity to SEQ ID NO: 1 or a nucleotide sequence encoding a polypeptide having sps and spp activity and at least about 80% sequence identity to SEQ ID NO: 2. As an example, a transformed host photosynthetic microorganism, such as a cyanobacterium, can comprise a nucleotide sequence having at least about 85%, at least about 90%, at least about 95%, or at least about 99% sequence identity to SEQ ID NO: 1, wherein the transformed host exhibits ASF, SPS, and/or SPP activity and/or accumulation of sucrose. As an example, a transformed host photosynthetic microorganism can comprise a nucleotide sequence encoding a polypeptide having at least about 85%, at least about 90%, at least about 95%, or at least about 99% sequence identity to SEQ ID NO: 2, wherein the transformed host exhibits ASF, SPS, and/or SPP activity and/or accumulation of sucrose. As another example, a transformed host photosynthetic microorganism can comprise a nucleotide sequence that hybridizes under stringent conditions to SEQ ID NO: 1 over the entire length of SEQ ID NO: 1, and which encodes an active SPS/SPP fusion (ASF) polypeptide. As a further example, a transformed host photosynthetic microorganism can comprise the complement to any of the above sequences.

In some embodiments, a sucrose phosphate synthase (sps) (see e.g., SEQ ID NO: 3 encoding sps gene and SEQ ID NO: 4 encoding SPS polypeptide), or homologue thereof, is engineered to be expressed or overexpressed in a transformed photosynthetic microorganism. For example, a photosynthetic microorganism can be transformed with a nucleotide having a sequence of SEQ ID NO: 3 so as to express sucrose phosphate synthase. As another example, a photosynthetic microorganism can be transformed with a nucleotide having at least about 80%, at least about 85%, at least about 90%, at least about 95%, or at least about 99% percent identity to SEQ ID NO: 3 encoding a polypeptide having sucrose phosphate synthase. As another example, a transformed host photosynthetic microorganism can comprise a nucleotide sequence encoding a polypeptide having at least about 85%, at least about 90%, at least about 95%, or at least about 99% sequence identity to SEQ ID NO: 4, wherein the transformed host exhibits SPS activity and/or accumulation of sucrose.

In some embodiments, sucrose phosphate phosphatase (spp) (see e.g., SEQ ID NO: 5 encoding spp gene and SEQ ID NO: 6 encoding SPP polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 5 so as to express sucrose phosphate phosphatase. As another example, a photosynthetic microorganism can be transformed with a nucleotide having at least about 80%, at least about 85%, at least about 90%, at least about 95%, or at least about 99% percent identity to SEQ ID NO: 5 encoding a polypeptide having sucrose phosphate phosphatase activity. As another example, a transformed host photosynthetic microorganism can comprise a nucleotide sequence encoding a polypeptide having at least about 85%, at least about 90%, at least about 95%, or at least about 99% sequence identity to SEQ ID NO: 6, wherein the transformed host exhibits SPP activity and/or accumulation of sucrose.

In some embodiments, a photosynthetic microorganism is engineered to express one or more of ASF, SPS, and/or SPP. For example, a photosynthetic microorganism, such as a cyanobacterium, can be engineered to express ASF and SPS; ASF and SPP; SPS and SPP; or ASF, SPS, and SPP.

Trehalose

Biosynthesis of trehalose can be accomplished through the catalytic action of two enzyme activities, trehalose phosphate synthase (tps) and trehalose phosphate phosphatase (tpp), functioning in sequence. Either or both of these activities can be engineered in a photosynthetic microorganism so as to result in accumulation of trehalose. Biosynthesis of trehalose does not naturally occur in some photosynthetic microorganisms, such as cyanobacteria.

In some embodiments, a trehalose phosphate synthase (tps) (see e.g., SEQ ID NO: 76 encoding tps gene and SEQ ID NO: 77 encoding TPS polypeptide), or homologue thereof, is engineered to be expressed or overexpressed in a transformed photosynthetic microorganism. For example, a photosynthetic microorganism, such as cyanobacterium, can be transformed with a nucleotide having a sequence of SEQ ID NO: 76 so as to express trehalose phosphate synthase. As another example, a photosynthetic microorganism can be transformed with a nucleotide having at least about 80%, at least about 85%, at least about 90%, at least about 95%, or at least about 99% percent identity to SEQ ID NO: 76 encoding a polypeptide having trehalose phosphate synthase. As another example, a transformed host photosynthetic microorganism can comprise a nucleotide sequence encoding a polypeptide having at least about 85%, at least about 90%, at least about 95%, or at least about 99% sequence identity to SEQ ID NO: 77, wherein the transformed host exhibits TPS activity and/or accumulation of trehalose.

In some embodiments, trehalose phosphate phosphatase (tpp) (see e.g., SEQ ID NO: 78 encoding tpp gene and SEQ ID NO: 79 encoding TPP polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 78 so as to express trehalose phosphate phosphatase. As another example, a photosynthetic microorganism can be transformed with a nucleotide having at least about 80%, at least about 85%, at least about 90%, at least about 95%, or at least about 99% percent identity to SEQ ID NO: 78 encoding a polypeptide having trehalose phosphate phosphatase activity. As another example, a transformed host photosynthetic microorganism can comprise a nucleotide sequence encoding a polypeptide having at least about 85%, at least about 90%, at least about 95%, or at least about 99% sequence identity to SEQ ID NO: 79, wherein the transformed host exhibits TPP activity and/or accumulation of trehalose.

Glucosylglycerol

In some embodiments, a glucosylglycerolphosphate synthase (gps) (see e.g., SEQ ID NO: 80 encoding gps gene and SEQ ID NO: 81 encoding GPS polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 80 so as to express glucosylglycerolphosphate synthase. As another example, a photosynthetic microorganism can be transformed with a nucleotide having at least about 80%, at least about 85%, at least about 90%, at least about 95%, or at least about 99% percent identity to SEQ ID NO: 80 encoding a polypeptide having glucosylglycerolphosphate synthase. As another example, a transformed host photosynthetic microorganism can comprise a nucleotide sequence encoding a polypeptide having at least about 85%, at least about 90%, at least about 95%, or at least about 99% sequence identity to SEQ ID NO: 81, wherein the transformed host exhibits GPS activity and/or accumulation of glucosylgycerol.

In some embodiments, glucosylglycerolphosphate phosphatase (gpp) (see e.g., SEQ ID NO: 82 encoding gpp gene and SEQ ID NO: 83 encoding GPP polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 82 so as to express glucosylglycerolphosphate phosphatase. As another example, a photosynthetic microorganism can be transformed with a nucleotide having at least about 80%, at least about 85%, at least about 90%, at least about 95%, or at least about 99% percent identity to SEQ ID NO: 82 encoding a polypeptide having glucosylglycerolphosphate phosphatase activity. As another example, a transformed host photosynthetic microorganism can comprise a nucleotide sequence encoding a polypeptide having at least about 85%, at least about 90%, at least about 95%, or at least about 99% sequence identity to SEQ ID NO: 83, wherein the transformed host exhibits GPP activity and/or accumulation of glucosylgycerol.

Mannosylfructose

In some embodiments, a mannosylfructose phosphate synthase (mps) (see e.g., SEQ ID NO: 84 encoding mps gene and SEQ ID NO: 85 encoding MPS polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 84 so as to express mannosylfructose phosphate synthase. As another example, a photosynthetic microorganism can be transformed with a nucleotide having at least about 80%, at least about 85%, at least about 90%, at least about 95%, or at least about 99% percent identity to SEQ ID NO: 84 encoding a polypeptide having mannosylfructose phosphate synthase. As another example, a transformed host photosynthetic microorganism can comprise a nucleotide sequence encoding a polypeptide having at least about 85%, at least about 90%, at least about 95%, or at least about 99% sequence identity to SEQ ID NO: 85, wherein the transformed host exhibits MPS activity and/or accumulation of mannosylfructose.

In some embodiments, mannosylfructose phosphate phosphatase (mpp) (see e.g., SEQ ID NO: 86 encoding mpp gene and SEQ ID NO: 87 encoding MPP polypeptide), or homologue thereof, is engineered to be expressed or overexpressed in a transformed photosynthetic microorganism. For example, a photosynthetic microorganism, such as a cyanobacterium, can be transformed with a nucleotide having a sequence of SEQ ID NO: 86 so as to express mannosylfructose phosphate phosphatase. As another example, a photosynthetic microorganism can be transformed with a nucleotide having at least about 80%, at least about 85%, at least about 90%, at least about 95%, or at least about 99% percent identity to SEQ ID NO: 86 encoding a polypeptide having mannosylfructose phosphate phosphatase activity. As another example, a transformed host photosynthetic microorganism can comprise a nucleotide sequence encoding a polypeptide having at least about 85%, at least about 90%, at least about 95%, or at least about 99% sequence identity to SEQ ID NO: 87, wherein the transformed host exhibits MPP activity and/or accumulation of mannosylfructose.

Molecular Engineering

Design, generation, and testing of the variant nucleotides, and their encoded polypeptides, having the above required percent identities to an asf sequence and retaining a required activity of the expressed protein and/or sugar accumulation phenotype is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide (e.g., asf, sps, spp, tps, tpp, gps, gpp, mps, or mpp) and/or polypeptide (e.g., ASF, SPS, SPP, TPS, TPP, GPS, GPP, MPS, or MPP) variants having, for example, at least 95-99% identity to the reference sequence described herein and screen such for phenotypes including disaccharide accumulation according to methods routine in the art. Generally, conservative substitutions can be made at any position so long as the required activity is retained.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Promoter

One or more of the nucleotide sequences discussed above (e.g., asf, sps, spp, tps, tpp, mps, mpp, gps, gpp) can be operably linked to a promoter that can function in the host photosynthetic microorganism. Where the host is cyanobacteria, preferably, the promoter can function efficiently in both cyanobacteria and a bacteria, such as E. coli. Promoter selection can allow expression of a desired gene product under a variety of conditions.

Promoters can be selected for optimal function in a photosynthetic microorganism host cell, such as a cyanobacterium, into which the vector construct will be inserted. Promoters can also be selected on the basis of their regulatory features. Examples of such features include enhancement of transcriptional activity and inducibility.

The promoter can be an inducible promoter. For example, the promoter can be induced according to temperature, pH, a hormone, a metabolite (e.g., lactose, mannitol, an amino acid), light (e.g., wavelength specific), osmotic potential (e.g., salt induced), a heavy metal, or an antibiotic. Numerous standard inducible promoters will be known to one of skill in the art.

In some embodiments, the promoter is a temperature inducible promoter. For example, the Lambda promoter is a temperature inducible promoter that can function in cyanobacteria. Surprisingly, the Lambda promoter functions at a temperature different than when utilized in E. coli. In E. coli, the Lambda promoter is most active at 42° C., a temperature above the normal viability range for cyanobacteria. Generally, in E. coli, the Lambda promoter has about a 5% to 10% increased expression from about 30° C. to 35° C. and at about 37° C. has about a 20% increased expression; but from about 37° C. to 42° C. provides about 100% increased expression. In cyanobacteria, the Lambda promoter is most active at around 30° C. to 35° C., an ideal growth temperature range for cyanobacteria and a range much lower than optimal expression of the Lambda promoter in E. coli. So, the Lambda promoter provides for effective expression of disaccharide biosynthetic activity in cyanobacteria.

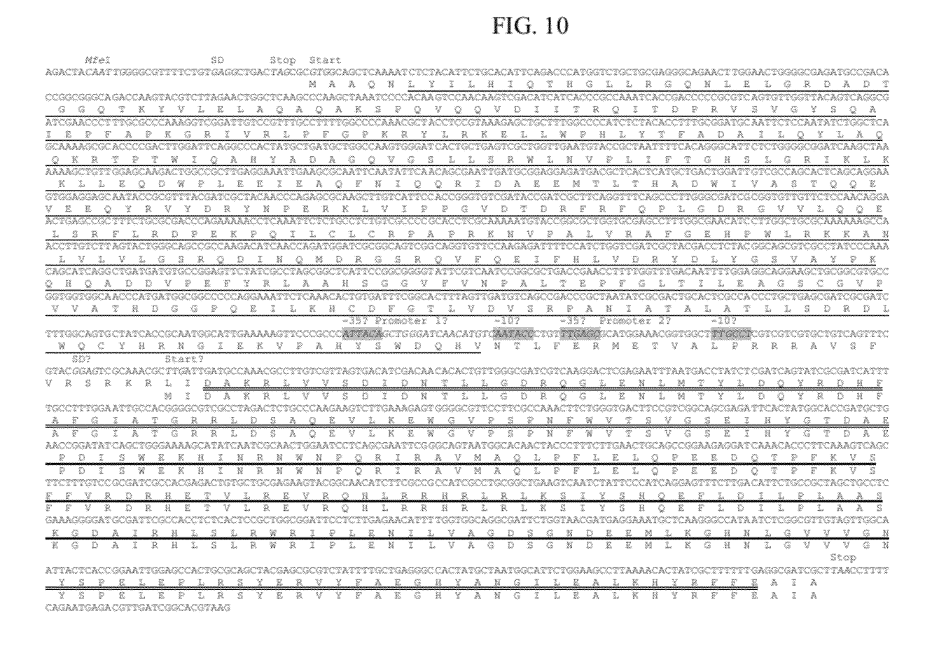

Examples of promoters that can be inserted into the plasmid include, but are not limited to, carB, nirA, psbAII, dnaK, kaiA, and λ_{PR} (see e.g., Example 6). In some embodiments, the promoter can function efficiently in both cyanobacteria and E. coli. In some embodiments, the asf coding region comprises a promoter with said coding region (see e.g., Example 8). For example, the asf coding region can comprise a promoter in front of the SPP domain of asf (see e.g., FIG. 10). Such an internal promoter can occur with or without a promoter at the start of the asf coding region.

The term “chimeric” is understood to refer to the product of the fusion of portions of two or more different polynucleotide molecules. “Chimeric promoter” is understood to refer to a promoter produced through the manipulation of known promoters or other polynucleotide molecules. Such chimeric promoters can combine enhancer domains that can confer or modulate gene expression from one or more promoters or regulatory elements, for example, by fusing a heterologous enhancer domain from a first promoter to a second promoter with its own partial or complete regulatory elements. Thus, the design, construction, and use of chimeric promoters according to the methods disclosed herein for modulating the expression of operably linked polynucleotide sequences are encompassed by the present invention.

Novel chimeric promoters can be designed or engineered by a number of methods. For example, a chimeric promoter may be produced by fusing an enhancer domain from a first promoter to a second promoter. The resultant chimeric promoter may have novel expression properties relative to the first or second promoters. Novel chimeric promoters can be constructed such that the enhancer domain from a first promoter is fused at the 5′ end, at the 3′ end, or at any position internal to the second promoter.

Constructs

Any of the transcribable polynucleotide molecule sequences described above can be provided in a construct. Constructs of the present invention generally include a promoter functional in the host photosynthetic microorganism, such as cyanobacteria, operably linked to a transcribable polynucleotide molecule for disaccharide biosynthesis (e.g., asf, sps, spp, tps, tpp, mps, mpp, gps, gpp), such as provided in SEQ ID NO: 1, 3, 5, 76, 78, 80, 82, 84, and 86, and variants thereof as discussed above.

Exemplary promoters are discussed above. One or more additional promoters may also be provided in the recombinant construct. These promoters can be operably linked to any of the transcribable polynucleotide molecule sequences described above.

The term “construct” is understood to refer to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. The term “vector” or “vector construct” is understood to refer to any recombinant polynucleotide construct that may be used for the purpose of transformation, i.e., the introduction of heterologous DNA into a host photosynthetic microorganism, such as a cyanobacterium.

In addition, constructs may include, but are not limited to, additional polynucleotide molecules from an untranslated region of the gene of interest. These additional polynucleotide molecules can be derived from a source that is native or heterologous with respect to the other elements present in the construct.

Plasmid

In some embodiments, a host photosynthetic microorganism, such as a cyanobacterium, is transformed with a plasmid-based expression system (see e.g., Example 5). Preferably the plasmid encoding the gene of interest comprises a promoter, such as one or more of those discussed above. For plasmid based transformation, preferred is a broad host range plasmid that enables function in both E. coli and cyanobacteria, which provides the advantage of working in a convenient fast growing well understood system (E. coli) that can be efficiently transferred to the final host (cyanobacteria). In some embodiments, plasmid based transformation and chromosomal integration are used in conjunction, where the plasmid protocol is used for design and testing of gene variants followed by chromosomal integration of identified variants.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

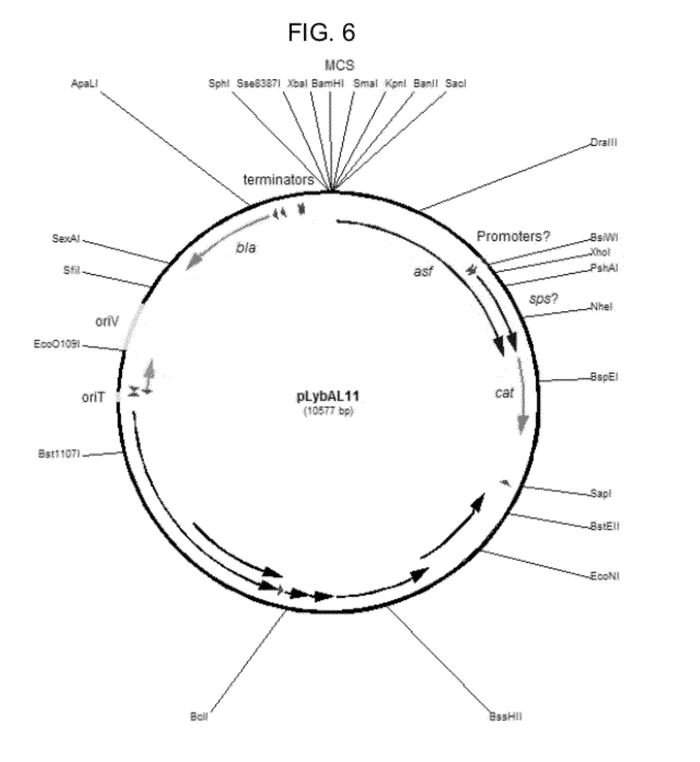

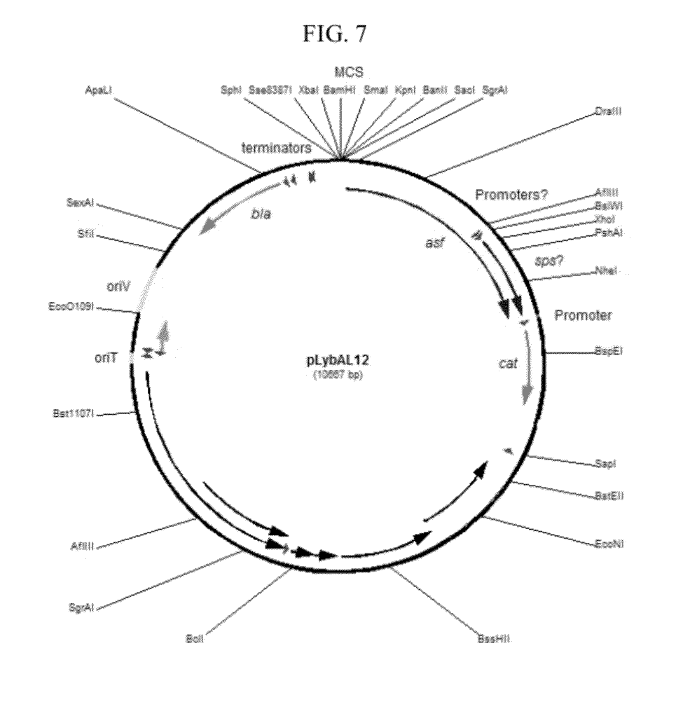

Provided herein are nucleotide sequences for plasmid constructs encoding sps, spp, and/or asf. Examples of plasmid constructs encoding sps, spp, and/or asf include, but are not limited to, pLybAL11 (SEQ ID NO: 19) (see e.g., FIG. 6) and pLybAL12 (SEQ ID NO: 20) (see e.g., FIG. 7). Also provided herein are nucleotide sequences for plasmid constructs encoding tps and tpp. Examples of plasmid constructs encoding tps and tpp include, but are not limited to, pLybAL23 (SEQ ID NO: 118). A skilled artisan will understand that similar constructs can be generated for biosynthetic genes necessary for accumulation of other disaccharides, such as glucosylglycerol and mannosylfructose.

In some embodiments, the transformed host photosynthetic microorganism comprises pLybAL11 (SEQ ID NO: 19) or pLybAL12 (SEQ ID NO: 20). In some embodiments, the transformed host photosynthetic microorganism comprises pLybAL23 (SEQ ID NO: 118). For example, a transformed cyanobacterium can comprise pLybAL11 (SEQ ID NO: 19), pLybAL12 (SEQ ID NO: 20), or pLybAL23 (SEQ ID NO: 118).

A plasmid construct comprising a disaccharide biosynthetic gene(s) can also include a promoter. Examples of plasmid constructs comprising sps, spp, and/or asf and a promoter include, but are not limited to, pLybAL7f (SEQ ID NO: 65); pLybAL8f, including kanamycin resistance (SEQ ID NO: 69); pLybAL13f (SEQ ID NO: 51), pLyAL13r (SEQ ID NO: 52), pLybAL14f (SEQ ID NO: 53), pLybAL14r (SEQ ID NO: 54), pLybAL15 (SEQ ID NO: 44), pLybAL16 (SEQ ID NO: 45), pLybAL17 (SEQ ID NO: 46), pLybAL18 (SEQ ID NO: 47), pLybAL19 (SEQ ID NO: 48), pLybAL21 (SEQ ID NO: 49), and pLybAL22 (SEQ ID NO: 50). Examples of plasmid constructs comprising tps and tpp and a promoter include, but are not limited to, pLybAL23 (SEQ ID NO: 118), pLybAL28 (SEQ ID NO: 121), pLybAL29 (SEQ ID NO: 122), and pLybAL30 (SEQ ID NO: 123). A skilled artisan will understand that similar promoter containing constructs can be generated for biosynthetic genes necessary for accumulation of other disaccharides, such as glucosylglycerol and mannosylfructose.

In some embodiments, the transformed host cyanobacterium comprises pLybAL7f (SEQ ID NO: 65); pLybAL8f (SEQ ID NO: 69); pLybAL13f (SEQ ID NO: 51), pLyAL13r (SEQ ID NO: 52), pLybAL14f (SEQ ID NO: 53), pLybAL14r (SEQ ID NO: 54), pLybAL15 (SEQ ID NO: 44), pLybAL16 (SEQ ID NO: 45), pLybAL17 (SEQ ID NO: 46), pLybAL18 (SEQ ID NO: 47), pLybAL19 (SEQ ID NO: 48), pLybAL21 (SEQ ID NO: 49), and pLybAL22 (SEQ ID NO: 50). In some embodiments, the transformed host cyanobacterium comprises pLybAL28 (SEQ ID NO: 121), pLybAL29 (SEQ ID NO: 122), pLybAL30 (SEQ ID NO: 123), and pLybAL23 (SEQ ID NO: 118).

Sugar Secretion

In various embodiments, a transformed disaccharide-accumulating photosynthetic microorganism can secrete the accumulated disaccharide from within the cell into its growth environment. Secretion of the disaccharide can be an inherent effect of transforming the photosynthetic microorganism to accumulate a disaccharide or the photosynthetic microorganism can be further engineered to secrete the disaccharide. For example, some cyanobacteria transformed to accumulate trehalose inherently secrete trehalose from the cell (see e.g., Examples 19-20). As another example, a cyanobacterium transformed to accumulate sucrose can be further engineered to secrete sucrose from the cell (see e.g., Example 16).

A host photosynthetic microorganism, such as a cyanobacterium, can be further engineered to secrete a disaccharide. In some embodiment, a transformed host photosynthetic microorganism is engineered to express a porin specific for the accumulated disaccharide. For example, a cyanobacterium engineered to accumulate sucrose can be further engineered to express a sucrose porin (see e.g., Example 16). In one embodiment, the transformed disaccharide-accumulating cyanobacterium comprises an scrY nucleic acid, such as SEQ ID NO: 94. In one embodiment, the transformed disaccharide-accumulating cyanobacterium comprises a nucleic acid encoding a scrY polypeptide, such as SEQ ID NO: 95. In one embodiment, the transformed disaccharide-accumulating cyanobacterium comprises a plasmid containing scrY, such as pLybAL32 (SEQ ID NO: 91). It is contemplated that a similar approach can be applied to other photosynthetic microorganisms or other target disaccharides.

Modulation of Sugar Degradation

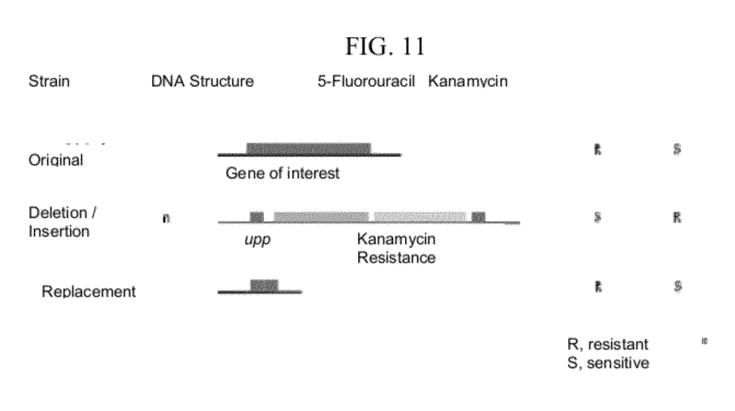

In some embodiments, a host photosynthetic microorganism, such as a cyanobacterium, is further engineered to improve disaccharide production by modulation of degradation activity (see e.g., Example 14). In some embodiments, an invertase homologue can be down-regulated or eliminated in a transformed photosynthetic microorganism. For example an invertase homologue from Synechocystis spp. PCC 6803 (nucleotide sequence SEQ ID NO: 70; polypeptide sequence SEQ ID NO: 71) can be down-regulated or eliminated in a transformed cyanobacterium. As another example, an invertase homologue from Synechococcus elongatus PCC 7942 (nucleotide sequence SEQ ID NO: 72; polypeptide sequence SEQ ID NO: 73) can be down-regulated or eliminated in a transformed cyanobacterium. In some embodiments, a sucraseferredoxin-like protein is down-regulated or eliminated in a transformed cyanobacteriuma. For example, a sucraseferredoxin-like protein from Synechocystis spp. PCC 6803 (nucleotide sequence SEQ ID NO: 74; polypeptide sequence SEQ ID NO: 75) (Machray G. C. et al. 1994. FEBS Lett 354, 123-127) can be down-regulated or eliminated in a transformed cyanobacterium. These genes can be deleted using the markerless deletion protocol described in, for example, FIG. 11 (see e.g., Examples 12-13) A similar approach can be taken for other disaccharides engineered to be accumulated in a cyanobacterium.

Other methods of down-regulation or silencing the above genes are known in the art. For example, disaccharide degradative activity can be down-regulated or eliminated using antisense oligonucleotides, protein aptamers, nucleotide aptamers, and RNA interference (RNAi) (e.g., small interfering RNAs (sRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem. Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several sRNA molecule design programs using a variety of algorithms are known to the art (see e.g., Cenix algorithm, Ambion; BLOCK-iT™ RNAi Designer, Invitrogen; sRNA Whitehead Institute Design Tools, Bioinformatics & Research Computing). Traits influential in defining optimal sRNA sequences include G/C content at the termini of the siRNAs, Tm of specific internal domains of the sRNA, sRNA length, position of the target sequence within the CDS (coding region), and nucleotide content of the 3′ overhangs.

In some embodiments, a host photosynthetic microorganism can be further engineered to promote disaccharide secretion from the cells. For example, a cyanobacterium can be further engineered to promote sucrose secretion from the cells (see e.g., Example 15-16). When in a low osmotic environment, the sucrose can be automatically expunged from the cells, as done with osmoprotectants by some organisms when transitioning from high to low salt environments (Schleyer, M., Schmidt, R. and Bakker, E. P. 1993. Arch Microbiol 160, 424-43; Koo, S. P., Higgins, C. F. and Booth, I. R. 1991. J Gen Microbiol 137, 2617-2625; Lamark, T., Styrvold, 0. B. and Strgim, A. R. 1992. FEMS Microbiol. Lett 96, 149-154). Sucrose porins can be engineered to be expressed in a transformed cyanobacterium (see e.g., Example 16). These genes can be cloned and transformed into cyanobacteria according to techniques described above. Such approaches can be adapted to other photosynthetic microorganisms.

In some embodiments, a host photosynthetic microorganism is transformed by stable integration into a chromosome of the host. For example, a host cyanobacterium can be transformed by stable integration into a chromosome of the host (see e.g., Examples 11-13). Chromosomal integration can insure that the target gene(s) is installed into the organism without risk of expulsion as sometimes occurs with plasmid-based gene expression. Chromosomal integration can also reduce or eliminate the need for antibiotics to maintain target genes.

Preferably, the strategy for chromosomal integration targets gene insertion into what is termed the upp locus on the chromosome (see e.g., Example 11-13). This site codes for the enzyme uracil phosphoribosyltransferase (UPRTase) which is a scavenger enzyme in pyrimidine biosynthesis. Using this strategy allows candidate selection by 5-fluorouracil (5-FU), which can eliminate non-integrated organisms. Segregation methods are generally used in cyanobacterial systems because these organisms contain multiple copies of their chromosomes (e.g., up to 12 for Synechocystis spp. PCC 6803 and 16 for Synechococcus elongatus PCC 7942). This strategy is particularly attractive for cyanobacteria, because this approach can avoid the use of traditional segregation techniques that rely on selective pressure and statistical integration for successful segregation. Using 5-FU as a screening agent can be more efficient because it can prevent growth for any organism that contains even a single active upp gene. In this manner, fully integrated candidates can be selected rapidly over fewer generation cycles compared to the processes required of traditional techniques.

Solid Phase Photosynthetic Bioreactor

Provided herein is a photobioreactor for culturing photosynthetic microorganisms comprising a solid phase cultivation support for the growth of photosynthetic microorganisms. A solid phase cultivation support, or solid cultivation support, or solid support, or the like, is generally understood to mean a cultivation support that is neither a liquid nor a gas. Although the support itself is a solid, the support structure may be selected so that it absorbs a liquid (e.g., growth media), a gas, or both. In certain preferred embodiments, as described more fully below, the solid support can absorb moisture for use by the microorganisms during cultivation.

Some embodiments of the photobioreactor utilize a solid cultivation support. Advantageously, the difficulty of providing adequate light exposures is alleviated, at least in part. Utilizing the aforementioned solid cultivation support in a photobioreactor can allow for cultivation and growth of photosynthetic microorganisms at cell densities greater than those of commercial-scale liquid phase bioreactors (e.g., cell densities in excess of 200 grams of dry biomass per liter equivalent). In addition, various embodiments of the photobioreactor described herein can be operated using less energy and more simply than conventional commercial-scale liquid phase photobioreactors.

Embodiments of the photobioreactor described herein provide additional benefits over conventional liquid phase photobioreactors. For example, liquid systems typically require special equipment to deliver adequate concentrations/amount of carbon dioxide to the photosynthetic microorganisms to support their growth and photosynthesis. In contrast, by growing the microorganisms on a solid cultivation support, carbon dioxide can be provided in a relatively simple, less costly manner, such as exposure to surrounding air. If additional carbon dioxide is desired, it can easily be delivered by, for example, adding it to the atmosphere (e.g., air) surrounding or in contact with the cultivation support. Another benefit is ease of transport. Liquid phase photobioreactors can be a pond (completely immobile) or bulky tanks or collections of tubing. In contrast, in various embodiments, the photobioreactor is flat and flexible, which allows for it or a multiplicity of them to be stacked, rolled up, folded, and/or configured in a similar manner for relatively easy transport. In various embodiments, the photobioreactor can be configured in a manner such that it is suspended from a system that allows for easy conveyance of one or more photobioreactors from one location to another. This portability may be utilized on a commercial scale to allow for efficient methods of handling and processing large numbers of photobioreactors in a continuous-type manner.

Various embodiments of the photobioreactor(s) described herein can support the growth a photosynthetic microorganism. The photosynthetic microorganism grown in the photobioreactor can be, for example, a naturally photosynthetic microorganism, such as a cyanobacterium, or an engineered photosynthetic microorganism, such as an artificially photosynthetic bacterium. Exemplary microorganisms that are either naturally photosynthetic or can be engineered to be photosynthetic include, but are not limited to, bacteria; fungi; archaea; protists; microscopic plants, such as a green algae; and animals such as plankton, planarian, and amoeba. Examples of naturally occurring photosynthetic microorganisms include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechoccus sp., Synechocystis sp., and/or Tolypothrix.

Preferably, the bioreactor is configured to support inoculation, growth, and/or harvesting of cyanobacteria transformed to accumulate a disaccharide, as described above.

The photobioreactor can be an open or a closed system, as described more fully below. In various embodiments, the photobioreactor includes a solid phase cultivation support, a protective barrier layer, and a suspension element. Some embodiments of the photobioreactor can contain a system for delivery and/or removal of gas, fluids, nutrients, and/or photosynthetic microorganisms. Delivery systems can be, for example, standard plumbing fixtures. Any of the various lines can include quick-connect plumbing fixtures. The photobioreactor can have a gas delivery line, which can deliver, for example, delivering carbon dioxide or normal atmospheric air. The photobioreactor can have a fluid delivery line. Preferably, the fluid delivery line connects to a trickle or drip system which conveys a fluid (e.g., water) to the solid phase cultivation support. The photobioreactor can have a nutrient delivery line. Formulation of a nutrient composition for the growth and maintenance of a photosynthetic microorganism is within the ordinary skill of the art. In some embodiments, the nutrient and fluid delivery lines can be combined, for example to supply a fluid-based nutrient mixture. In some embodiments, the fluid delivery line or the nutrient delivery line can be a spray device for distributing a liquid medium over the growth surface. In such spray devices, the photobioreactor is large enough to accommodate, for example, a spray device between an outer layer, such as a barrier layer, and the solid phase cultivation support. Usually, nutrients are supplied in a water-based composition. It can be advantageous to provide for different water delivery line(s) and nutrient delivery line(s) so as to provide for independent control of moisture and nutrient levels. The photobioreactor can have a product harvest line so as to provide for collection of photosynthetic microorganisms and/or liquid suspended/soluble products. The photobioreactor can have an inoculation line so as to provide for inoculation of photosynthetic microorganisms. In some embodiments, the fluid, nutrient, and/or inoculation lines can be combined.

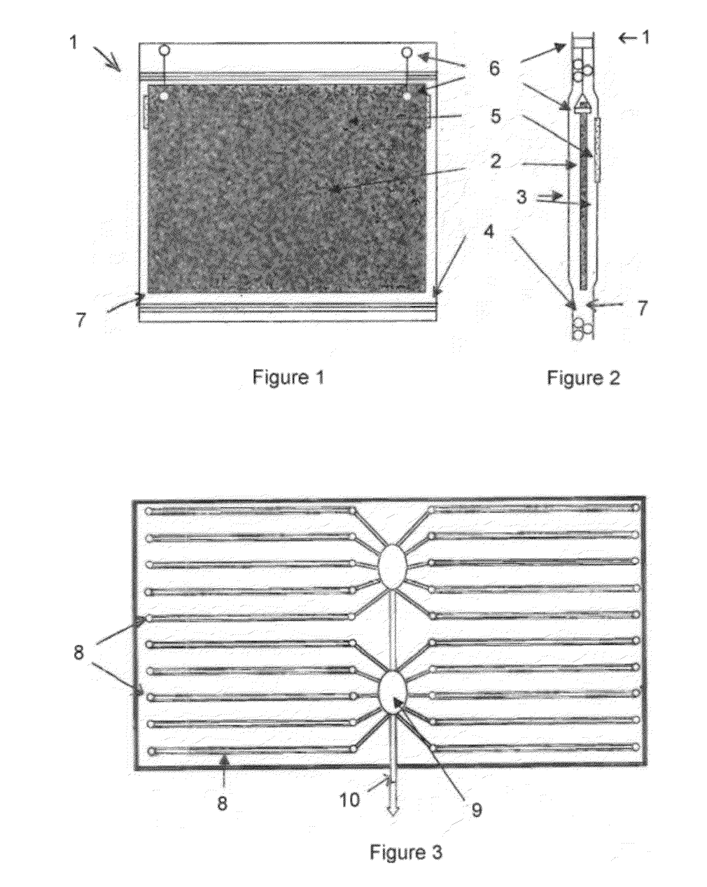

One embodiment of a solid-phase photobioreactor is depicted in FIG. 1 (front view) and FIG. 2 (side view). In these embodiments, a solid phase cultivation support 2 is enclosed by protective barrier 7. FIG. 2 shows that the solid cultivation support is between protective barrier layers 3 that comprise the protective barrier 7. The solid cultivation support 2 provides the surface upon which photosynthetic microorganisms are cultivated. The protective barrier layers 3 that make up the protective barrier 7 are transparent to allow actinic radiation to reach the surface of the solid cultivation support 2 to support the growth of photosynthetic microorganisms. Resealable closures 4 allow for a protective barrier 7 that is releasably sealed. Exchange of gases and vapor occurs through a selective panel 5 of material that is incorporated into the protective barrier 7. The photobioreactor 1 can be suspended by support elements 6 to allow for a vertical or non-horizontal orientation.

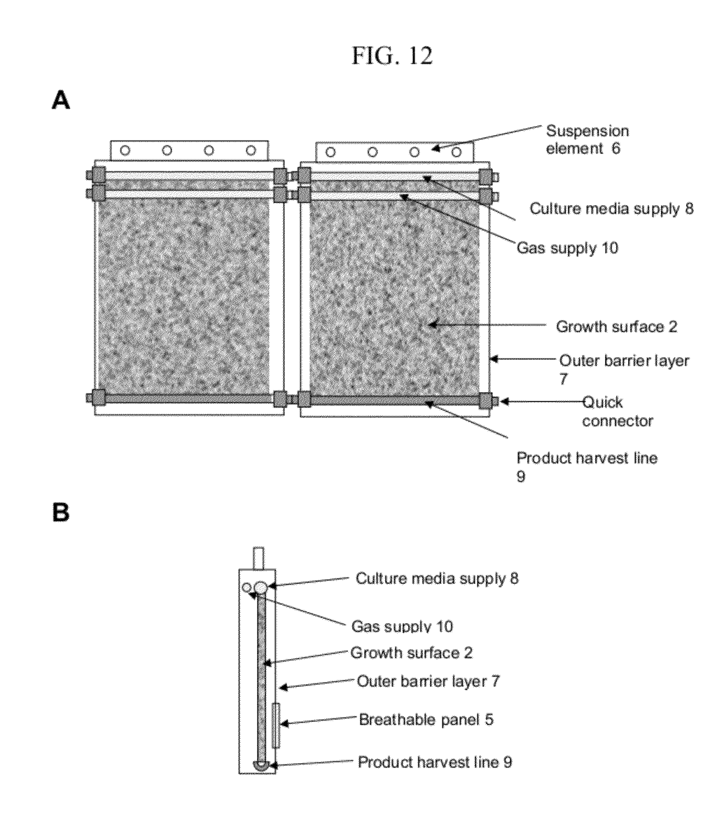

Another embodiment of a solid-phase photobioreactor is depicted in FIG. 12A (front view) and FIG. 12B (side view). The reactor 1 can be designed in a segmented format, which can aid in servicing and minimizes potential contamination of the surface and/or plumbing. Each segment can be connected to the reactor through plumbing (e.g., quick connect type plumbing) of the various supply and product harvest lines. The reactor can be supported by a suspension element 6 from, for example, rails, which allows the reactor 1 to hang in space and aid in rapid servicing of each segment. The outer protective barrier 7 can be a transparent material that enables light penetration facilitating photosynthesis on the growth surface 2, while preventing environmental contamination and moisture loss from evaporation. The growth surface 2 can be composed of a material that retains moisture, supplies nutrients, removes products, and/or enables high density growth of photosynthetic microorganisms. The growth surface 2 can be serviced by plumbing that provides continuous feeding/product harvest from the surface by liquid culture media. The media tubing 8 can be a porous hose that seeps liquid to the surface 2, which can percolate through the growth surface 2 by gravity. The liquid can be harvested at the bottom of the reactor by a harvesting tube 9, which collects products and excess liquid media for transport from the reactor 1. Gases, such as carbon dioxide and air, can be supplied to the reactor by a gas dispersion tube 10. The gas supply tube 10 can provide a positive pressure environment and is expected to supply gases necessary for growth in a controlled, efficient manner. The gas supply line 10 can also assist in minimizing moisture loss by humidifying incoming gas streams. Excess gas from the reactor can be vented by a breathable panel 5 (on the reverse side, not shown) that is a porous material that allows for gas passage but minimizes or eliminates environmental contamination. Contamination is expected to be minimized by the positive pressure configuration of the reactor 1 through filtration of the incoming gas delivered by the supply line 10. Positive pressure can also prevent contamination from the environment by providing an inside out pathway for gas flow.

In the embodiment depicted in FIG. 12B, features of the reactor 1 are depicted in an orientation relative to the growth surface. The breathable panel 5 allowing for excess gas to escape the reactor 1 can be located toward the bottom of the device to provide a path for gas to migrate across the growth surface 2. Location of the breathable panel 5 on the bottom of the barrier surface 7 also minimizes or prevents the possibility of carbon dioxide segregation and build up resulting from its higher density relative to air. The dimensions of the breathable panel 5 can be determined based on gas flow rate requirements for optimal growth on the cultivation surface 2.

Solid Phase Cultivation Support

The solid phase cultivation support of a photobioreactor as described herein provides a surface on and/or in which a photosynthetic microorganism can grow. Preferably, the solid phase cultivation support comprises a material that provides or facilitates the provision and/or retention of moisture and/or nutrients to the organisms, so as to promote and sustain growth. Embodiments of the invention are not limited to the type or strain of photosynthetic microorganisms that can be cultivated. One of ordinary skill in the art will recognize that the amount of moisture and the amount and composition of nutrients desirable for cell growth will vary with the type or strain of photosynthetic microorganism and the application for which it is to be grown. Materials (or the substances contained within or on those materials) that may have a deleterious effect on the growth of photosynthetic microorganisms are generally avoided.

A single photobioreactor can be used to cultivate a single type or multiple types or strains of photosynthetic microorganisms. Further, the solid cultivation support can comprise material(s) such that it is suitable for a single cultivation cycle or multiple cycles of cultivation, with or without sterilization between cultivation cycles. Still further, a photobioreactor can be configured to cultivate a single type or strain of microorganism or multiple types or strains of microorganisms on a single or multiple solid supports. In some embodiments, instead of an axenic culture, a community of different photosynthetic microorganisms, or a community of photosynthetic and non-photosynthetic microorganisms, can be grown together simultaneously on one cultivation support. A single photobioreactor can also comprise multiple cultivation supports. Thus in another embodiment, multiple cultivation supports within a single protective barrier can cultivate one or more types or strains of photosynthetic microorganisms simultaneously.

The solid cultivation support preferably comprises a relatively porous material. A relatively porous material generally has increased surface area and can retain and/or absorb more moisture than a relatively non-porous material. Also preferred is a solid cultivation support that has a textured or topographical surface(s). A textured or topographical surface can enhance cell density compared to a relatively non-textured or smooth surface. Although the choice of support material and surface topography are typically selected to enhance the adhesion of microorganisms to the support, it generally is desirable that the organisms not so tightly adhere so as to impede their removal or harvest. In some embodiments, the solid cultivation support comprises a material suitable for adhesion and growth of microorganisms. In some embodiments, the solid cultivation support comprises a material that reduces or eliminates biofilm formation.

The solid-phase supports of the photobioreactors described herein are believed to be different from solid supports that have been utilized in the art (e.g., the most commonly used solid phase support for the growth of microorganisms is agar). Agar is generally cast into rigid forms, such as a petri dish, and used while therein to maintain its physical integrity because agar tends to break or tear when subjected to minimal levels of stress, strain, or both. In contrast, various embodiments of the cultivation support is sufficiently strong and durable that it can be used in a photobioreactor while maintaining its physical integrity without the need of a stronger, more durable “frame”. Or stated another way, the prior art involved a sufficient portion of the weak agar support in contact with a substantially stronger, more durable material (e.g., a petri dish) such that a composite is formed. Thus, the solid-phase supports of various embodiments of the photobioreactor are suitable in themselves for the cultivation of microorganisms and are sufficiently strong and durable.

Other desirable physical characteristics and/or operation parameters of the solid-phase support are described below. For example, the support can be relatively flat and rigid (like a plate) or it may consist of a multiplicity of flat and rigid sections flexibly connected by, e.g., hinges, springs, wires, threads, etc. Suitable rigid materials include, but are not limited to, various metals, polymers, ceramics, and composites thereof. The rigid materials preferably have surface topographies that enhance the adherence of the photosynthetic microorganisms thereto. Further, the rigid materials may be formed with a desired level of porosity to enhance the ability to deliver moisture and/or nutrients to the photosynthetic microorganisms. Still further, the rigid materials may be coated with absorbent or super absorbent polymer formulations (see below). Alternatively, the support may consist essentially of flexible material, such as a fabric. Fabrics for use in a solid-phase support include, but are not limited to, cotton, polyester, and/or cotton polyester blends, optionally coated with absorbent or super absorbent polymer formulations. Flexibility of the cultivation support can be greatly advantageous because it allows for the cultivation support to be folded, twisted, draped, or rolled for storage, transport, or handling.

In addition, the solid-phase cultivation support is preferably structurally stable at elevated temperatures (e.g., about 120° C. and above), such as would be typically encountered during autoclave sterilization, and will not melt like agar. Thus, in one embodiment, the cultivation support may be sterilized by autoclaving and then placed within the protective barrier of the invention. In another embodiment, the cultivation support can be placed within the protective barrier, and the entire photobioreactor may then be autoclaved. Although autoclaving is one method for sterilization, one of skill in the art will recognize that any other appropriate method of sterilization may be utilized.

The solid cultivation support of the present invention can comprise or be made of any material appropriate for supporting the growth of photosynthetic microorganisms. For example, the support may be composed of natural materials, modified natural materials, synthetic materials, or any combination thereof. Natural materials can include, but are not limited to cotton, wool, processed woven plant fibers, and natural polysaccharides (e.g., agar, starches, cellulosics). Modified natural materials can include, but are not limited to, chemically modified plant fibers such as nitrocellulose or cellulose esters, in addition to natural fibers co-woven or blended with polyester or polyamide fibers. Synthetic materials can include, but are not limited to, fibers composed of nylon, fiberglass, polysiloxanes, polyester, polyolefins, polyamide, copolyester polyethylene, polyacrylates, or polysulfonates. Further examples of solid cultivation support materials include wire mesh, polyurethane foams, polyethylene foams, vitreous carbon foams, polyester/polyethylene foams, polyimide foams, polyisocyanate foams, polystyrene foams, and polyether foams, or combinations thereof.

In various embodiments, the solid cultivation support is a fabric. The fabric can be formed by methods such as, but not limited to, weaving, knitting, felting, and the bonding or cross-linking of fibers or polymers together. The construction of the fabric can be loose or open. Alternatively, the fabric can be tightly constructed. That said, fabrics that have a significant texture, surface area, topographical variability, and/or roughness may provide more mechanical bonding or adherence of the photosynthetic microorganisms to the cultivation support and thus may be preferable, especially in embodiments wherein the photobioreactor is handled, transported, or otherwise moved during the process for inoculating the support with, and/or growing and/or harvesting the organisms. Preferably, in most applications the adherence of the organisms to the substrate should not be so great as to unduly hinder their removal during a harvesting operation. Still further, the ability of a fabric to retain moisture and/or nutrients for use by the organisms can be controlled by selecting fibers that are generally hydrophobic, hydrophilic, or a mixture of such fibers. These properties allow for moisture and/or nutrients dissolved therein to be retained and/or transported by the solid support so that they are available to the microorganisms growing on the surface.

The properties of the cultivation support, especially moisture and/or nutrient retention, can be enhanced by coating the support with a material selected to enhance photosynthetic microorganism growth. For example, the cultivation support can be coated with agar or a super absorbent polymer such as modified cellulose ester, acrylate or acrylate/polyamine copolymer blends. These coating materials are typically able to absorb and retain greater than 10 to 100 times their dry weight in water. In some embodiments, these materials are formulated such that they would retain their superabsorbent properties in the presence of ionic culture media components. The coating material can coat the surface of the cultivation support, or the fibers of a fabric if used, or both. In one embodiment, a swatch of terrycloth serving as the cultivation support is coated in agar. When a solid cultivation support is coated as such, the “surface” of the cultivation support includes the surface of the coating if photosynthetic microorganisms attach to such. To keep the cultivation support thin, pliable, and light, the coating is preferably thin, for example, no greater than about 100 microns. However, thicker coatings can also be used depending on the application desired, or on the combination of solid cultivation support and coating material selected.

The solid-phase cultivation support can be a composite, layered structure. The solid-phase cultivation support can comprise at least two layers arranged so as to be adjacent. Multiple layers of the solid-phase cultivation support can be coupled, such as by bonding, stitching, adhesive, compression, or any other suitable means. The various layers can each independently be selected from among the several materials discussed above. For example, the solid-phase cultivation support can comprise a first material layer of fabric bonded to a second material layer of synthetic foam. An another example, the solid-phase cultivation support can comprise a first material layer of synthetic foam bonded to a second material layer of synthetic foam of the same or different density. Preferably, the solid-phase cultivation support is a composite, layered structure comprising at least a first layer, which is composed of a high surface area growth material, and a second layer, which is composed of a permeable type material.

In addition to supplying moisture, nutrients, and a surface for attachment, the cultivation support can provide a surface for capturing actinic radiation. Thus, in some embodiments, the dimensions of the solid cultivation support are sheet-like. That is, the depth of the support is small relative to the length and width of the support. In one embodiment, the cultivation support is a sheet-like layer between film-like layers of a protective barrier. Such a flat bioreactor can be suspended like a flat panel. In another embodiment, just the cultivation support is suspended like a curtain enclosed by the outer barrier of the photobioreactor. A thin sheet of a traditional solid phase support such as agar would easily rip apart, and would likely not be able to be suspended as such. Therefore, it is preferable that the solid cultivation support alone be able to maintain its integrity when suspended, even when saturated with liquid.

As shown herein, a fabric with a terrycloth-type weave can provide a suitable solid support (see e.g., Example 1). One of skill in the art will understand that other natural, modified-natural, and synthetic materials may also be acceptable. Terrycloth provides many of the attributes believed to be desirable in a solid support of the present invention. For example, it is flexible, and not prone to tearing, ripping, breaking, or cracking when handled in accordance with non-destructive techniques (e.g., bending, folding, twisting, or rolling) under conventional conditions (e.g., temperature). Likewise, terrycloth is typically not prone to tearing, ripping, or breaking when modestly stretched (even when saturated with liquid). Additionally, terrycloth tends to be highly textured because it is composed of the many loops of fibers. This provides a large amount of surface area for the attachment of microorganisms thereby increasing the amount of microorganisms that can be grown on a support of any given size. Further, a cotton terrycloth typically absorbs at least about three times its own weight, which allows for moisture and any nutrients dissolved therein to be retained by the fabric support so that they are available to the microorganisms growing on the surface of the support. Thus, various embodiments provide for a solid cultivation support that is thin or sheet-like in dimension, able to support its own wet weight while suspended, flexible, pliable, absorbent, highly textured, or any combination thereof.

The above-described supports can be, and in many applications preferably are, used repeatedly and more preferably for so long as they are structurally sound and provide a surface adequate to support the growth of the microorganisms disposed of after a single use thereby reducing operational costs and waste. That said, there can be certain applications in which single-use supports would be desirable, such as cultivation of recombinant photosynthetic microorganisms useful in producing pharmaceutical products such as small organic molecules or therapeutic proteins and peptides. To reduce the costs of such single-use supports and in view of the fact that that they will not be reused, such supports need not be as durable and therefore can be made or constructed using methods and/or materials that are less costly and less durable. For example, supports comprised of paper fibers similar to that of paper towels may be appropriate.

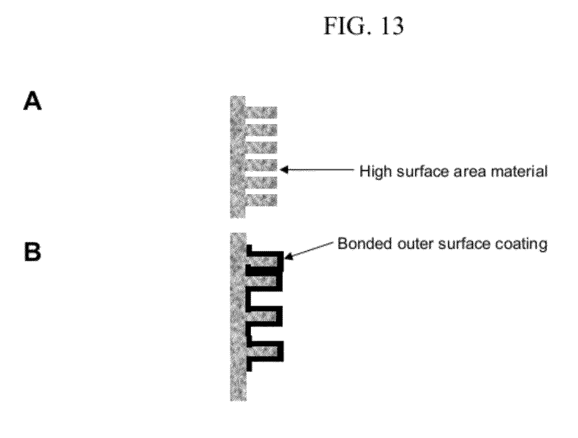

Several embodiments of a solid phase cultivation support are depicted in FIG. 13. The solid phase cultivation support material depicted in FIG. 13A is a single material that can provide sustainable surface for organism growth, access to moisture and nutrients, point of organism attachment, and/or removal of cultivation products. The material can allow for liquid percolation and equilibrium diffusion to exchange nutrients, moisture, and products between the surface and organisms. The rendering of the structure configuration is an example of a high surface area material, which can be optimized for dimension and shape. The solid phase cultivation support material depicted in FIG. 13B is a hybrid material that is composed of multiple layers of materials, each having specific functions for the growth surface. The base layer can be a porous material that efficiently allows for supply of nutrients and moisture as well as removal of products that are percolated through the material. The base material can also provide physical support for the growth surface. The outer layer(s) is expected to be attached to the base layer and can be optimized to provide point of attachment for the organisms. The surface layer can achieve more control of the surface growth environment in terms of surface area and compatibility with the cultivated organism.

The cultivation support can be designed in accordance with the disclosures of U.S. Provisional Application Ser. No. 61/085,797, filed 1 Aug. 2008, U.S. Provisional Application Ser. No. 61/018,798, filed 3 Jan. 2008, PCT International Publication No. WO2010/048525, published 29 Apr. 2010, PCT International Publication No. WO2007/076449, published 5 Jul. 2007, and/or PCT International Publication No. WO2004/076356, published 10 Sep. 2004, each of which is incorporated herein in its entirety by reference.

Protective Barrier

A photobioreactor as described herein can comprise a barrier that protects the solid cultivation support and growth surface from contamination and/or moisture loss. At the same time, the photobioreactor provides for actinic radiation, either sunlight or artificial light, and carbon dioxide reaching the photosynthetic microorganisms. In various embodiments, the photobioreactor comprises at least one solid support and a protective barrier for the cultivation of photosynthetic microorganisms.

Protection from Physical Handling and/or Contamination

To prevent contamination, a protective physical barrier can at least partially cover the solid cultivation support. In certain embodiments, the physical barrier can enclose the cultivation support. The protective barrier can also control, at least in part, the loss of the moisture from the support and/or the atmosphere within the photobioreactor to the atmosphere outside the photobioreactor. One of skill in the art will recognize that the protective barrier can be constructed from any of numerous types of materials depending on the embodiment of the invention desired.

The protective barrier can completely enclose the cultivation support. If the protective barrier is permanently sealed, the barrier must be breached, cut, torn, or the like to access the cultivation support within. Thus, in some embodiments, access is provided through the protective barrier to the cultivation support and the surface on which the microorganisms are grown.

In preferred embodiments, the protective barrier is releasably sealed. The releasable seal can be any of a number of closure types including, but not limited to zipper-type closures such as found in Ziploc® storage bags (SC Johnson Company), hook-and-loop type fasteners (e.g., Velcro USA, Inc.), twist ties, zipties, snaps, clips, pressure sensitive adhesive backed surfaces, and all art recognized equivalents thereto. A complete seal, however, is not necessarily required; and it may be more efficient not to completely seal the outer barrier to allow for easier access to the cultivation support.

The photobioreactor can comprise a single cultivation support or multiple cultivation supports within a protective barrier. In some embodiments, a single cultivation support is enclosed within a single protective barrier. For example, a plastic bag may form a protective barrier within which a single solid cultivation support is enclosed (see e.g., FIG. 1). In other embodiments, a single protective barrier may enclose multiple solid cultivation supports. For example, a greenhouse-type structure may form a protective barrier within which multiple solid cultivation supports are enclosed.

Transmission of Actinic Radiation

The photobioreactor can provide for transmission of actinic radiation, either sunlight or artificial light, to the photosynthetic microorganisms. But the protective barrier of the invention need not necessarily be transparent to light. Some embodiments can comprise a cultivation support enclosed within a non-transparent protective barrier if a sufficient light source for the growth of photosynthetic microorganisms is provided within. It may be desirable, simpler, more economical, and the like to provide a transparent barrier to utilize sunlight, for instance, as a light source.

Preferred embodiments provide for a transparent barrier comprising a material such as, but not limited, glass or any type of transparent or generally visible light transmitting polymer such as polyethylene, acrylic polymers, polyethylene terephthalate, polystyrene, polytetrafluoroethylene, or co-polymers thereof, or combinations thereof. The transparent barrier can be selected from materials that are durable and not prone to ripping, tearing, cracking, fraying, shredding, or other such physical damage. The transparent barrier material can be selected for its ability to withstand autoclave sterilization or other exposure to temperature extremes. Further, the transparent barrier materials can be selected to withstand prolonged exposure to sunlight or other radiation without discoloring or deteriorating. One of skill in the art will recognize that certain coatings or formulations that resist photooxidation can be particularly useful. In addition, infrared reflecting or absorbing coatings can be selected to reduce and/or otherwise regulate the buildup of temperature within the photobioreactor of the invention.

One of skill in the art will recognize that the thickness of the transparent barrier material will vary depending on mechanical properties of scale. For example, the transparent barrier material may be of an industrial/marine type plastic about 10 mil thick or it may be of the type used in a household plastic bag, i.e., around 2 mil thick. In one embodiment, the transparent barrier material is thin and flexible. For example, the transparent barrier material can be less than about 10 mil.

In some embodiments, the barrier forms a protective layer or film covering the two sides of a thin, flexible, solid cultivation support. The assembled photobioreactor of this embodiment would be flexible, and could be bent, rolled, folded, twisted, or the like for storage, transport, conveying, or handling. In another embodiment, the transparent barrier material is rigid. For example, the barrier can be a glass greenhouse. Most likely, the thickness of the greenhouse glass would preferably be consistent with building practices but it is possible that it could be altered. The photobioreactor of such an embodiment would be for practical purposes immovable, but multiple solid supports could be handled, transported, conveyed and the like within the confines of one protective, transparent barrier.

Although a protective barrier can be selected to provide sufficient light for the growth of photosynthetic microorganisms, it is not necessary that the entire barrier be transparent. Thus, in some embodiments, portions of the barrier, such as one or more edges, are made from a non-transparent material. The non-transparent material can be composed of materials including, but not limited to polyethylene fiber material (Tyvek®), polytetrafluoroethylene filtration media, cellulosic filter material, fiberglass filter material, polyester filter material and polyacrylate filter material, and combinations thereof. The non-transparent material can be selected for durability. In such an embodiment, a transparent portion of the barrier would be further protected from tearing, ripping, fraying, shredding, and the like by a durable, non-transparent portion. In one embodiment, a non-transparent portion provides or comprises an attachment structure and/or reinforcement for suspending the photobioreactor by further comprising mounting or attachment points (e.g., holes, loops, hooks, grommets, or other art equivalent device, opening or, recess) and/or or a mechanism for securing the photobioreactor to a structure. Although it is not required that any such mounting points, etc., be located in or on the non-transparent portion, they can be contained within or on a non-transparent portion of the barrier, within or on a transparent portion of the barrier, or within or on a non-transparent and a transparent portion of the barrier. The attaching structure may also be contained within or on, or pass through, the solid cultivation support.

In some embodiments, the device has a discernable front side and back side. The front side of this device is meant to face a light source, and thus the portion of the barrier on the front side is preferably transparent, while the portion of the protective barrier on the side facing away from the light source is not necessarily transparent.

The transmission of actinic radiation can be provided in accordance with the disclosures of U.S. Provisional Application Ser. No. 61/085,797, filed 1 Aug. 2008, U.S. Provisional Application Ser. No. 61/018,798, filed 3 Jan. 2008, PCT International Publication No. WO2010/048525, published 29 Apr. 2010, PCT International Publication No. WO2007/076449, published 5 Jul. 2007, PCT International Publication No. WO2004/076356, published 10 Sep. 2004, each of which is incorporated herein in its entirety by reference.

Provision of Gas Exchange

During photosynthesis, photosynthetic microorganisms consume carbon dioxide and release oxygen. A photobioreactor as described herein can provide carbon dioxide sufficient for a desired amount of photosynthesis to occur. One way to supply carbon dioxide to the inside of the photobioreactor is to allow direct gas exchange between the air inside and the air surrounding the photobioreactor. For example, holes, vents, windows, or other such openings can be provided in the protective barrier so that the system is open to the surrounding atmosphere.

But such an open configuration may not be desirable when contamination of the photosynthetic microorganisms is a concern. To address this concern, the protective barrier can completely seal off the solid support or supports enclosed within from the outside air. In such an embodiment, the desired concentration of carbon dioxide can be maintained by introducing it into the enclosure. For example, one of skill in the art would recognize that plumbing or tubing from a tank of compressed carbon dioxide would allow for carbon dioxide to be mixed into the air enclosed within the photobioreactor. In addition, it is known that the emissions from factories, industrial plants, power plants, or the like can be harnessed as a source of carbon dioxide for photosynthetic microorganisms, thus reducing carbon emissions. In one embodiment, a gas supply line can provide carbon dioxide to the growth surface local area.

It may be desirable, simpler, more economical, and the like to provide a selective barrier that is gas permeable to utilize atmospheric carbon dioxide. Thus, some photobioreactor embodiments provide for a selective barrier that allows gas and vapor exchange between the environment enclosed within the protective barrier and the surrounding air, while still providing a sealed physical barrier against contamination. Such barrier can be at least partially gas/vapor permeable (e.g., much less permeable than conventional textile fabrics, higher than that of plastic films, and/or similar to that of coated papers), thus allowing the exchange of gases such as carbon dioxide and oxygen but is additionally at least partially and preferably considered to be impermeable to solids and liquids. In some embodiments, the photobioreactor can contain a semi-permeable barrier layer and a gas supply line to maintain an elevated carbon dioxide concentration in the area around or near the growth surface.

In some embodiments, a selective barrier can have an average pore size or diameter of no greater than about 10 micrometers and a gas exchange rate that is at least about 5 and no greater than about 10,000 Gurley seconds (a Gurley second or Gurley is a unit describing the number of seconds required for 100 cubic centimeters of gas to pass through 1.0 square inch of a given material at a given pressure differential). Therefore, in addition to allowing gas exchange, the selective barrier can prevent loss of moisture from the enclosed system.

The selective barrier portion of the protective barrier can be composed of any appropriate polymer-based material, such as spunbonded olefin barriers. Spunbonded olefin barriers (very fine polyethylene fibers) with various properties are readily available from DuPont under the brand name Tyvek®. Such materials are particularly advantageous because of their combination of physical properties, i.e., they tend to resist the transmission of liquids such as water yet they have a sufficiently high degree of gas/vapor permeability; they are relatively strong, absorb little or no moisture, are rip-resistant, have a significant degree of elasticity, and are highly flexible. Spunbonded olefin can exceed 20,000 cycles when tested on an MIT flex tester (TAPPI method T-423). In addition, they are inert to most acids, bases and salts although a prolonged exposure to oxidizing substances, such as concentrated nitric acid or sodium persulfate, will cause some loss of strength. Spunbonded olefin barriers have good dimensional stability in that sheet dimensions tend to change less than 0.01% between 0 and 100% relative humidity at constant temperature. Certain products meet the requirements of Title 21 of the United States Code of Federal Regulations (21 CFR 177.1520) for direct food contact applications. They also have excellent mold and mildew resistance; and are of a neutral pH. Unfortunately, however, their UV resistance is not exceptional. That said, at least one to three months of useful outdoor life can usually be expected. Additionally, their UV resistance can be improved with opaque coatings or by including UV inhibitors in the polymer fibers. Additionally, because the spunbonded oelefins produced to date are opaque, the portion of the protective barrier that would comprise such material is preferably not situated and/or so extensive as to compromise the cultivation of the photosynthetic microorganisms.

In particular, spunbonded olefin can be produced in “hard” and “soft” structure types. Type 10, a “hard,” area-bonded product, is a smooth, stiff non-directional paper-like form. Types 14 and 16 are “soft,” point-bonded products with an embossed pattern, providing a fabric-like flexible substrate. Type 14 styles (or the equivalent thereof) can be used, for example, where barrier, durability, and breathability are required. Type 16 styles are pin perforated with 5-20 mil (0.13-0.51 mm) holes, giving them much higher air and moisture permeability, additional softness, and greater flexibility and drape than Type 14 styles, but at the expense of lower tear strength and barrier properties. Thus, the particular properties of the selective barrier can be customized by selecting one or more types of spunbonded olefin products.

Other examples of selective polymer barriers include, but are not limited to nylon, polysulfone, polytetrafluoroethylene, cellulosic, fiberglass, polyester and polyacrylate membranes and filter material, and combinations thereof.

The entirety of the protective barrier need not be gas permeable to provide for a barrier that is sufficiently selective for the growth of photosynthetic microorganisms. Only a portion of the protective barrier sufficient to allow for adequate gas exchange need be gas permeable. In one embodiment, the selective portion is a panel of the protective barrier (see e.g., FIG. 1). The size and placement of the selective panel in relation to the area of the support surface can be altered to achieve a desired amount of gas exchange for a particular application without unduly hindering the cultivation of the microorganisms. One of skill in the art will recognize that the percentage of the area of the outer barrier composed of the gas permeable selective material will depend on the gas permeability rate of the material. In fact, because the gas permeable portion will still allow the transport of water vapor across it, in various embodiments, the size of the gas permeable portion of the protective barrier is selected so as to allow for sufficient transport of oxygen and carbon dioxide while minimizing the loss of moisture.

The provisioning of gas exchange can be designed in accordance with the disclosures of U.S. Provisional Application Ser. No. 61/085,797, filed 1 Aug. 2008, U.S. Provisional Application Ser. No. 61/018,798, filed 3 Jan. 2008, PCT International Publication No. WO2010/048525, published 29 Apr. 2010, PCT International Publication No. WO2007/076449, published 5 Jul. 2007, PCT International Publication No. WO2004/076356, published 10 Sep. 2004, each of which is incorporated herein in its entirety by reference.

Suspension and Conveyance System

Photobioreactors described herein can be configured for large scale production and/or harvesting through, for example, integration into a handling and conveyance system. FIG. 3 shows an above view of an exemplary design of a photobioreactor farm for handling large numbers of photobioreactors in a continuous process. The photobioreactors or cultivation panels (not individually shown) are attached to conveyor systems 8. The conveyor systems 8 move the cultivation panels along their paths. Multiple conveyor systems converge at centrally located inoculation and harvesting centers 9. Thus, the cultivation panels are moved into the inoculation and harvesting centers 9 where they can be processed (e.g., harvested and/or inoculated) and then the panels are moved away from the centers following inoculation and during the period of cultivation of the biomass. The panels are then moved back towards the centers during the latter period of cultivation prior to harvesting, eventually arriving back at the centers with mature biomass for harvest. The cycle is then repeated. Harvested biomass can be transported through a pipeline 10 for further processing. The capacity of the photobioreactor farm can be increased by adding additional conveyor systems or additional inoculation and harvest centers to form large arrays dedicated to biomass production.

The suspension and conveyance of the cultivation support can be provided in accordance with the disclosures of U.S. Provisional Application Ser. No. 61/085,797, filed 1 Aug. 2008, U.S. Provisional Application Ser. No. 61/018,798, filed 3 Jan. 2008, PCT International Publication No. WO2010/048525, published 29 Apr. 2010, PCT International Publication No. WO2007/076449, published 5 Jul. 2007, PCT International Publication No. WO2004/076356, published 10 Sep. 2004, each of which is incorporated herein in its entirety by reference.

Suspension of PhotoBioreactor

To supply light to photosynthetic microorganisms, a favored embodiment of the photobioreactor is one in which the cultivation support is thin and sheet-like. When oriented horizontally, the efficient utilization of floor space tends to decrease, therefore in certain embodiments of the invention the cultivation support is oriented non-horizontally, preferably substantially vertically, or more preferably vertically. Nevertheless, the cultivation support may be oriented in essentially any manner so long as a sufficient amount of actinic radiation can reach the microorganisms. Thus, when the photobioreactor is of the type where the protective barrier forms a closely associated film or layer around the solid support, a preferred orientation of the entire photobioreactor is vertical, but any orientation is acceptable. To be clear, the aforementioned orientations (e.g., vertical, horizontal, substantially vertical, non-horizontal, etc.) are relative to the floor or ground beneath the cultivation support, assuming that the floor or ground is horizontal.

Various structures, scaffolding, stands, racks, etc. may be used to hold or suspend a cultivation support or an entire photobioreactor in a desired orientation. In particular, the cultivation support and/or the protective barrier can be suspended from, or attached to a rope, line, hook, cable, track, rail, chain, shelf, pole, tube, scaffold, stand, beam or any other such structure capable of suspending the solid cultivation support and/or photobioreactor. Multiple cultivation supports and/or photobioreactors may be suspended from a common structure, like sheets hanging from a clothes line. The cultivation support(s) and/or photobioreactor(s) may be suspended statically, or in a manner that allows for their movement. The position of the holes, loops, hooks, or the like will preferably distribute the weight of the cultivation support and/or photobioreactor substantially evenly.

Suspension of the photobioreactor or cultivation support, especially in a vertical orientation, is space efficient and may provide advantages in handling. However, the bioreactor or cultivation support of the invention need not be suspended. For example, in certain embodiments of the present invention, the cultivation support is sufficiently rigid that if oriented non-horizontally, vertically, or substantially vertically (e.g., by securing or placing its base to/on a surface, in an embodiment in which the support is like a rigid plate, panel, grid, etc.) it can support its own weight and will remain so oriented. In another embodiment, the protective barrier is free standing, such as a greenhouse, and multiple cultivation supports are suspended and/or free-standing within.

Suspension of the photobioreactor and/or cultivation support, especially in a vertical orientation, is space efficient and may provide advantages in handling. However, the bioreactor or cultivation support of the invention need not be suspended. For example, in certain embodiments of the present invention, the cultivation support is sufficiently rigid that if oriented non-horizontally, vertically, or substantially vertically (e.g., by securing or placing its base to/on a surface, in an embodiment in which the support is like a rigid plate, panel, grid, etc.) it can support its own weight and will remain so oriented. In another embodiment, the protective barrier is free standing, such as a greenhouse, and multiple cultivation supports are suspended and/or free-standing within.

Conveyance

Also described herein is a system for conveying photobioreactors, cultivation supports within the protective barrier of a photobioreactor, or some combination thereof from one location to another. The ability to transport a photobioreactor and/or cultivation support can be advantageous for a variety of reasons. For example, it may allow for optimizing their position(s) for receiving light, and for maintaining a desired temperature or gas content. The transportability can be particularly advantageous when multiple photobioreactors or cultivation supports are to be subject to discrete steps, such as inoculating, cultivating, inducing, and/or harvesting, because it is likely to be more efficient to move the photobioreactors or cultivation supports to several assigned locations in a continuous-type process instead of transporting the necessary materials and equipment to stationary photobioreactors or cultivation supports.

Thus, the growing surface, whether the cultivation support alone, or the cultivation support enclosed in a protective barrier, can be conveyed, even after inoculation. One of skill in the art will be familiar with numerous types of conveyor systems frequently used in industrial applications. The conveyance system is not limited to any particular type so long as it is capable of moving one or more photobioreactors or cultivation supports. One skilled in the art will recognize that the type of attachment between the photobioreactor or cultivation support and the conveyor system will vary with the type of conveyance system employed and will be selected to work cooperatively with any mounting points that are part of the cultivation support and/or the protective barrier. Although it is envisioned that the cultivation support(s) or photobioreactor(s) will be conveyed in a mechanized manner powered by one or more motors (e.g., through the action of a chain and gears), it is also possible for them to be conveyed with human effort (e.g., by simply pushing suspended bioreactors that are attached to a rail by a bearing mechanism that slides along the rail).

A conveyor system that suspends photobioreactor(s) and/or cultivation support(s), especially in a vertical orientation, is space efficient and may provide advantages in handling. But the conveyor system need not rely on suspending photobioreactor(s) or cultivation support(s). For example, a photobioreactor may move along on top of the conveyor system, such as by sliding over a roller conveyor. In one embodiment, the conveyor system may move photobioreactors comprising a cultivation support enclosed in a protective barrier. Alternatively, the protective barrier of a photobioreactor may be a large enclosure protecting one or more conveyor systems moving multiple cultivation supports.

Photobioreactor Farm

For large scale applications, it may be impractical to construct a single cultivation support of sufficient size. Thus is provided use of two or several or tens or hundreds or thousands or more cultivation supports to cultivate photosynthetic microorganisms in a photobioreactor “farm.” These cultivation supports can all reside within a single protective barrier, thus comprising a single photobioreactor, or multiple cultivation supports may be part of multiple photobioreactors. In either case, it can be beneficial to organize the multiple photobioreactors or cultivation supports within a photobioreactor farm for ease and efficiency of handling and processing. It can also be beneficial to organize their arrangement to maximize the amount of energy captured from a light source such as the sun. Such organization can consist of arranging numerous photobioreactors or cultivation supports in an orderly fashion such as, but not limited to, rows, columns, concentric circles, in grids, radiating outward from a central point, and so forth.

In various embodiments, the farm comprises multiple photobioreactors or cultivation supports suspended from a common structure such as a track, rail, chain, line, or the like. In further embodiments, the structure is part of a conveyor system and the photobioreactors or cultivation supports move along the path of the conveyor system from one location to another.

A photobioreactor farm can comprise one or an arrangement of multiple conveyor systems handling numerous photobioreactors or cultivation supports. Such an arrangement could be scaled up to comprise two or several or tens or hundreds or thousands or more conveyor systems together handling two or several or tens or hundreds or thousands or more photobioreactors or cultivation supports. In addition to the conveyor system(s), a photobioreactor farm can include defined areas, stations, or centers for performing steps such as inoculating, cultivating, inducing, and/or harvesting photosynthetic microorganisms. Such centers can be the location of specialized equipment for performing certain steps. The paths of the conveyor systems can bring the photobioreactors or cultivation supports to such centers where a particular step is performed. The photobioreactor or cultivation support can then be moved along to the next area or center in the sequence. Different photobioreactors or cultivation supports along the conveyor system can reside at different centers along the path and thus be subject to different steps simultaneously. In one embodiment, the path of the conveyor system is a loop. Once a photobioreactor or cultivation support completes one round of steps in the cultivation process, it can repeat the process. Allowing for some units to be damaged or otherwise eventually needing replacement, essentially the same set of photobioreactors or solid cultivation supports can be used repeatedly.

In a further embodiment, cultivation and harvest can occur at the same or nearly the same location. This location is termed an inoculation and harvest center (see e.g., FIG. 3). Inoculation of the photobioreactors and/or solid cultivation supports occurs at the inoculation and harvest center. The conveyor system forms a loop that then transports the photobioreactors or cultivation supports away from the inoculation and harvest center. The photobioreactors or cultivation supports then travel along the path of the conveyor system for an amount of time sufficient for the desired amount of cell growth. The conveyor system then returns the photobioreactors or cultivation supports back to the inoculation and harvest center for harvest. Multiple conveyor systems can share a common inoculation and harvest center from which they radiate out from. If even more capacity is needed, a photobioreactor farm can comprise multiple inoculation and harvest centers handling the photobioreactors or cultivation supports from multiple conveyor systems. Although increased efficiencies may be realized, it is not necessary that the location of inoculation and of harvest be the same or nearly the same location.

Methods of Using a Photobioreactor

Cultivation of Photosynthetic Microorganisms

A solid phase photobioreactor, as described herein, can be used for cultivating photosynthetic microorganisms. Photosynthetic microorganisms that can be grown in the solid phase photobioreactor include, but are not limited to, a naturally photosynthetic microorganism, such as a cyanobacterium, or an engineered photosynthetic microorganism, such as an artificially photosynthetic bacterium. Exemplary microorganisms that are either naturally photosynthetic or can be engineered to be photosynthetic include, but are not limited to, bacteria; fungi; archaea; protists; microscopic plants, such as a green algae; and animals such as plankton, planarian, and amoeba. Examples of naturally occurring photosynthetic microorganisms that can be grown in the bioreactor include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechoccus sp., Synechocystis sp., and/or Tolypothrix.

Preferably, the photosynthetic microorganisms grown in the solid phase photobioreactor comprise cyanobacteria. The cyanobacterium grown in the bioreactor can be any photosynthetic microorganism from the phylum Cyanophyta. The cyanobacterium grown in the bioreactor can have a unicellular or colonial (e.g., filaments, sheets, or balls) morphology. Preferably, the cyanobacterium grown in the bioreactor is a unicellular cyanobacterium. Examples of cyanobacteria that can be grown in the bioreactor include, but are not limited to, the genus Synechocystis, Synechococcus, Thermosynechococcus, Nostoc, Prochlorococcu, Microcystis, Anabaena, Spirulina, and Gloeobacter. Preferably the cyanobacterium grown in the bioreactor is a Synechocystis spp. or Synechococcus spp. (e.g., Synechococcus elongatus PCC 7942 (ATCC 33912) and/or Synechocystis spp. PCC 6803 (ATCC 27184)). More preferably, the photosynthetic microorganism grown in the bioreactor is a transgenic photosynthetic microorganism engineered to accumulate a disaccharide, as disclosed herein.

A solid cultivation support of a photobioreactor can be inoculated with a photosynthetic microorganism, along with addition of moisture and other components including, but not limited to, nutrients, salts, buffers, metals, nitrogen, phosphate, sulfur, etc. The photobioreactor can then be releasably sealed with the cultivation support within the protective barrier. The sealed photobioreactor can be placed, for example by suspending it, in a location and manner to allow for control of illumination and temperature. The placement can be static, or the photobioreactor can be moved, such as to ensure maximum exposure to the sun's radiation over the course of a day. The photosynthetic microorganisms can be cultivated for a desired amount of time. One of skill in the art will recognize that the length of time will vary according to the type of microorganism and the density of cell growth desired. For example, for certain strains of cyanobacteria, a cultivation period that is within the range of about four to about seven days can provide a yield of cells that is within the range of about 50 to about 250 grams of dry biomass per liter equivalent. Following a period for cultivation, the releasable seal can be opened and the photosynthetic microorganisms can be harvested.

As used herein, “grams of dry biomass per liter equivalent” is a unit determined by calculating the average depth of the biomass layer (e.g., about 150 microns) growing on the cultivation surface and multiplying that value by the length and the width of the cultivation surface. This calculation provides a volume. The weight of the collected biomass from the cultivation surface can then be correlated to the volume and expressed as “grams of dry biomass per liter equivalent.”

The cultivation of photosynthetic microorganisms can be accomplished in accordance with the disclosures of U.S. Provisional Application Ser. No. 61/085,797, filed 1 Aug. 2008, U.S. Provisional Application Ser. No. 61/018,798, filed 3 Jan. 2008, PCT International Publication No. WO2010/048525, published 29 Apr. 2010, PCT International Publication No. WO2007/076449, published 5 Jul. 2007, PCT International Publication No. WO2004/076356, published 10 Sep. 2004, each of which is incorporated herein in its entirety by reference.

Method of Continuous Cultivation

Greater efficiencies can be realized if the process of cultivating photosynthetic microorganisms were to be made continuous, for example, like an assembly line. Instead of requiring the equipment and capacity to handle a large amount of biomass all at once that then sits idle in between batches, a continuous system would require less total capacity, but would utilize that capacity more efficiently through continuous operation. By dividing cultivation into smaller but more numerous components, the components can be organized in a spatially continuous arrangement. Different discrete steps of the overall production process can then occur simultaneously. After a cultivation component is subjected to a process step, the component moves forward in the process while another component replaces it in that step. Therefore, production of the end product would not be limited to the maturation of a large batch, but can occur regularly as individual components complete the assembly line-like process. Further, following the completion of one round of the process, the components can immediately start the process over and do so repeatedly.

More specifically, continuous cultivation relates to methods of using conveyable photobioreactors or cultivation supports for cultivating photosynthetic microorganisms in a continuous manner. Continuous or continuous process is understood as the spatial relationship that can allow the photobioreactors or solid cultivation supports to progress from one step of the cultivation process to another. Alternatively, it is possible for a single large structural support to be utilized in a continuous process. Specifically, the support can be a loop of material (e.g., terry cloth fabric) that is made to travel along a circuit (e.g., like a conveyor belt that is arranged preferably vertically). The end result is that biomass production can be achieved regularly as multiple photobioreactors or solid cultivation supports finish the process sequentially and repeatedly. This type of process presents opportunities in large scale applications for increased efficiencies over producing biomass in large, but infrequent batches.

In a preferred embodiment, the continuous spatial relationship is along the path of a conveyor system. The manner of operation is analogous to an assembly line. Such a conveyor system can operate in a number of ways. For example, the conveyor system can operate without interruption while moving the photobioreactors or cultivation supports from one location to another. In such an embodiment, inoculation, harvesting, and the like occur while the photobioreactors or cultivation supports are in motion. Alternatively, the conveyor system can stop to allow for steps to be performed, and then resume to move the photobioreactors or cultivation supports to the location of the next step. Further, the conveyor system can operate without interruption, and the photobioreactors or cultivation supports can be detached from the movement of the conveyor system for processing, and then reattached to re-enter into the stream of conveyance. One skilled in the art will realize that other permutations of this general theme are also possible.

In one embodiment of a method of continuous cultivation, multiple photobioreactors are inoculated at one location along the conveyor system. The conveyor system then moves the photobioreactors to an area where cultivation of the photosynthetic microorganisms occurs. During this portion of conveyance, the photobioreactors can be positioned to allow for optimal illumination to promote growth and photosynthesis. Next, the photobioreactors would arrive at a location where the photosynthetic microorganisms can be harvested. The photobioreactors can then return along the path of the conveyor system to the point of inoculation to begin the process again. To improve efficiency, the time between when the photobioreactors leave the location of inoculation and arrive at the location of harvest can be made to coincide with the time it takes for the desired amount of growth of the photosynthetic microorganisms to occur. The steps of the process are not limited to inoculation, cultivation, and harvest; additional steps can include inducement of the cells to synthesize a desired product or sterilization. Although the above embodiment describes a system of conveyable photobioreactors, it will be appreciated that the same type of continuous cultivation can be practiced within a single protective barrier to convey and process multiple solid cultivation supports.

Method of Producing Fermentable Sugars

One technology that can benefit from the ability to more efficiently grow photosynthetic microorganisms is the production of biomass for alternative fuels such as ethanol or biodiesel. Relative to plants currently grown to produce biomass such as corn, sugarcane, soybeans, canola, jatropha, and so forth, photosynthetic microorganisms, such as cyanobacteria, produce biomass at a much faster rate, which may lead to much greater productivity. In addition, direct production of disaccharides by microorganisms avoids much of the extensive energy-intensive pre-processing of using plant biomass to produce fermentable sugar. Further, the use of phototrophic microorganisms instead of plants can lead to higher yields of fermentable sugars without soil depletion, erosion, and diversion of the food supply. Relative to other microorganisms, preference is given to phototrophic microorganisms because their sources of carbon (CO₂) and energy (light) can be supplied from the environment, making them far less expensive to cultivate. In addition, phototrophic microorganisms can be utilized to consume carbon emissions from industrial processes, thus providing further benefits to the environment.

One obstacle to producing high quantities of fermentable sugars from photosynthetic microorganisms is that they generally consume produced carbohydrates rather than accumulating them. While some sugars, such as sucrose or trehalose, are not utilized as a primary carbon source by photosynthetic microorganisms, there are mechanisms for slow assimilation. In spite of reprocessing mechanisms, such material can accumulate without being metabolized. If the organism is engineered appropriately, the assimilation mechanism can be inactivated, which enables high yields of sugars to be produced.

Provided herein is a method for producing fermentable sugars, especially disaccharide sugars, by photosynthetic microorganisms. Examples of fermentable sugars include, but are not limited to, sucrose, trehalose, glucosylgycerol, and mannosylfructose. Preferably, the fermentable sugar is sucrose or trehalose. The method can be adapted to occur in a continuous manner to improve the cost effectiveness of production.

Various embodiments of this method can be practiced using a photosynthetic microorganism capable of synthesizing fermentable sugars. Some embodiments harness and control the natural phenomena of osmo- and matric water protection for the generation of fermentation feedstocks. In one embodiment, synthesis of fermentable sugars is inducible. In another embodiment, synthesis of fermentable sugars can be modified by genetic manipulation to be produced constitutively.

Fermentable sugar-producing photosynthetic microorganisms are preferably cyanobacteria. In some embodiments, a cyanobacterium accumulates a disaccharide according to inducible endogenous pathways. In some embodiments, a transgenic cyanobacterium accumulates a disaccharide according to engineered exogenous pathways. Both endogenous and exogenous pathways are discussed in further detail above.

Preferably, the transgenic photosynthetic microorganisms are one or more of those discussed above.

Two non-limiting examples of strains of cyanobacteria capable of accumulating a disaccharide are Synechococcus elongatus PCC 7942 and Synechocystis sp. PCC 6803. Naturally occurring Synechococcus elongatus PCC 7942 synthesizes sucrose upon exposure to salt concentrations of up to about 700 mM, its tolerance limit. When glucosylglycerol biosynthesis is blocked by deletion of the agp gene, Synechocystis sp. PCC 6803 produces sucrose as its osmoprotectant upon exposure to salt concentrations up to its tolerance limit which may approach 900 mM. In some embodiments, salt induction can be accomplished by introducing aerosolized saline solution applied directly to the cultivation surface. One advantage of this process is application can be controllably introduced along the growing surface depending on growth time of the cultivar thereby balancing accumulation of biomass and production of a disaccharide such as sucrose.

For producing fermentable sugars, the photosynthetic microorganisms can be cultured and grown on a solid medium or in a liquid or gel medium. Culture and growth of photosynthetic microorganisms are well known in the art. Except as otherwise noted herein, therefore, culture and growth of photosynthetic microorganisms can be carried out in accordance with such known processes. For example, a transgenic cyanobacteria engineered to accumulate a disaccharide can be cultured and grown in a liquid medium. The accumulated sugar can be isolated from such liquid medium if excreted from the cell. The accumulated sugar can be isolated from photosynthetic microorganisms harvested from the liquid medium. In one embodiment, a transgenic cyanobacteria engineered to accumulate trehalose, as discussed above, is cultured and grown in a liquid medium. Trehalose secreted from the transgenic cyanobacteria can be isolated directly from the liquid medium. In one embodiment, a transgenic cyanobacteria engineered to accumulate sucrose, as discussed above, is cultured and grown in a liquid medium. Sucrose can be isolated directly from engineered cyanobacteria harvested from the liquid medium. In one embodiment, a transgenic cyanobacteria engineered to accumulate and secrete sucrose, as discussed above, is cultured and grown in a liquid medium. Sucrose secreted from the transgenic cyanobacteria can be isolated directly from the liquid medium.

Preferably, photosynthetic microorganisms are cultivated to a relatively high cell density of at least about 50 grams of dry biomass per liter equivalent prior to induction. Such relatively high cell densities can be achieved using a solid phase photobioreactor, as described herein. Disaccharide (e.g., sucrose) production can then be initiated/induced by treating the accumulated biomass with defined concentrations of suitable salt compounds effective at altering the activity of water in the culture media as measured by solution conductivity. In a further preferred embodiment, sodium chloride is the salt used. Following an appropriate response time period (e.g., at least about 1 hour to no greater than about 48 hours), the sucrose laden cells can be harvested and processed to isolate and recover the sucrose produced. Typically, an appropriate response period is within the range of at least about 5 hours to no greater than about 24 hours. More typically, the appropriate response period is within the range of at least about 10 hours to no greater than about 20 hours.

In one embodiment, the majority of disaccharide (e.g., sucrose, trehalose, glucosylglycerol, mannosylfructose) synthesized accumulates within the cells. In another embodiment, the disaccharide is secreted by the cells which can then be recovered from the photobioreactor. Regardless of whether the disaccharide is within the cells or secreted, the disaccharide can be obtained using any appropriate harvesting process including, but not limited to, an aqueous spray wash applied to the cultivation surface. The wash comprising cells and/or disaccharide can be collected and processed to isolate and recover the disaccharide.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Solid Phase Photobioreactor

A static prototype device was constructed composed of a 2 mil polyethylene barrier layer with a Ziploc® resealable closure. A 60 sq. cm breathable panel was incorporated into one surface, and a 225 sq. cm woven cotton fabric cultivation support surface was placed inside. The device was sterilized by treatment with 70% volume aqueous ethanol followed by drying of the device at 50° C. with a stream of sterile filtered air. 30 ml of sterile BG-11 culture media was absorbed onto the cultivation support followed by inoculation of the growing surface with a pre-culture of Synechococcus elongates PCC 7942. using an aerosol applicator. The preculture was grown in BG-11 media at 26° C. for 2 days prior to inoculation. The photobioreactor was placed in an incubation chamber maintained at 33° C. and illuminated at 300 microeinsteins with cool white fluorescent lamps. After 2 days, the reactor displayed active growth of organisms and was allowed to continue growth for an additional 2 days whereupon the reactor was removed from the incubator and the growth surface washed with deionized water. The water was removed by evaporation to afford 254 mg dry weight biomass.

### Example 2

### Production of Sucrose by Photosynthetic Microorganisms

The following is a prophetic example to illustrate a method for production of sucrose by photosynthetic microorganism in combination with a photobioreactor. At least one photobioreactor, for example a photobioreactor of the current invention such as described in Example 1 or Example 3, may be run for approximately 4-7 days with either Synechocystis sp. PCC6803. or engineered Synechocystis sp. at a temperature range of between about 15 and 40° C., under illumination of between about 60 and 300 microeinsteins, and carbon dioxide concentration of between about 0.2 and 15 volume %. Following the initial cultivation period the growth surface may be treated with an aqueous salt solution in the concentration range of between about 0.01 and 1.5 M, more preferably between about 0.2 and 0.9 M, using an aerosol spray. The cultivation may be allowed to continue for approximately an additional one to two days to allow sucrose production. The growth surface may then be harvested by washing the surface with deionized water. In a further embodiment the wash water is sterile fresh cultivation media and the washing stringency is such that between about 70 and 90% of the cell mass is collected. The biomass remaining on the cultivation support may then be allowed to continue growth as a subsequent cycle. It is anticipated that the yield for these cultivations should be between about 200 and 600 mg dry biomass depending on the growth surface material and organism employed.

### Example 3

### Solid Cultivation Support Coated with an Absorbent Polymer

The growth surface of a static photobioreactor of the type described in Example 1 was prepared by dip coating the sterile dry surface of the material with a heated solution of sterile 1.5 weight percent agar dispersed in BG-11 culture media. The coated growth surface was allowed to cool and harden upon which the surface was inserted into a sterilized protective barrier to form a photobioreactor device and inoculated with Synechococcus sp. grown in preculture as described in Example 1. Cultivation and harvesting were performed essentially as described in Example 1.

### Example 4

### ASF Gene Target

Biosynthesis of sucrose in cyanobacteria was explored through modulation of sucrose phosphate synthase (sps) and sucrose phosphate phosphatase (spp) activities. Such activities are already present in many cyanobacteria for acclimation to osmotic and matric water stress (see e.g., Lunn, J. E. 2002. Plant Physiol 128, 1490-1500).

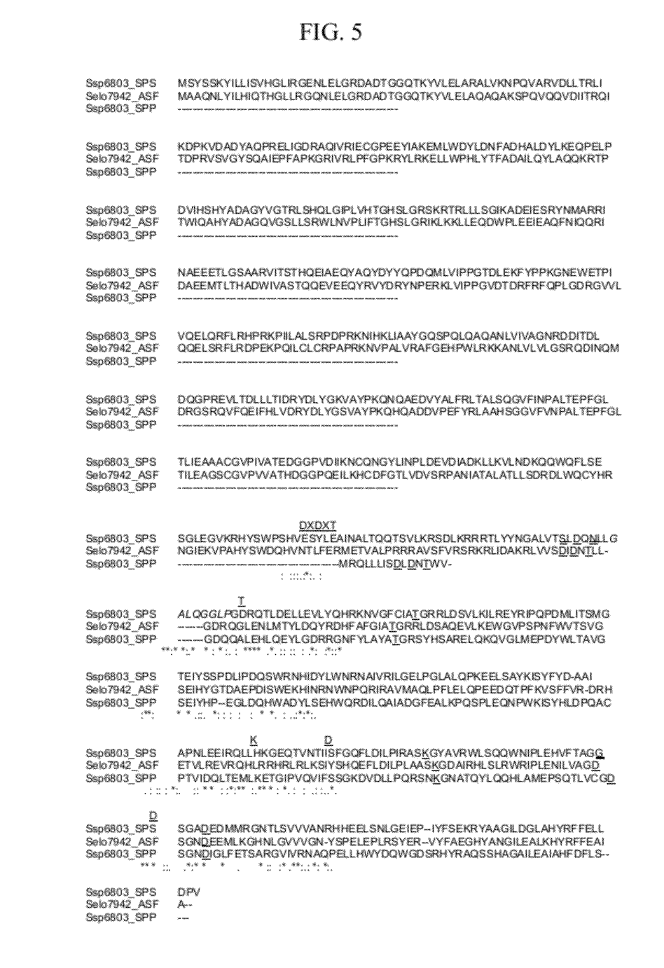

Lunn, J. E. (2002. Plant Physiol 128, 1490-1500) analyzed the genomic organization of the sps and spp genes of several organisms, including Synechocystis spp. PCC 6803 and Synechococcus elongatus PCC 7942. Lunn proposed that the sucrose phosphate synthase (SPS) of Synechocystis spp. PCC 6803 (SEQ ID NO: 3) has an inactive sucrose phosphate phosphatase (SPP-like) domain and a distinct SPP activity. The SPP-like domain has a high level of identity with the spp, but is missing many of the conserved active site residues of the haloacid dehalogenase (HAD) superfamily. While no work has yet been done on Synechococcus elongatus PCC 7942, Lunn proposed that both activities are contained within a single enzyme. An alignment of these enzymes is shown in FIG. 5.

Searches of the Synechococcus elongatus PCC 7942 genome did not reveal a distinct sps gene elsewhere on the chromosome. The Synechococcus elongatus PCC 7942 enzyme (SEQ ID NO: 2) was utilized so as to avoid the necessity of multiple gene expression. While the gene from PCC 7942 has been termed sps, because it is a single enzyme fusion bearing both SPS and SPP activities, it was termed asf for active SPS/SPP fusion (SEQ ID NO: 1) (see below for further information on the possible expression of a distinct SPP enzyme.)

There are two approaches to expressing the Synechococcus elongatus PCC 7942 asf gene product (SEQ ID NO: 2).

The first approach is a plasmid-based expression system built upon the broad host range vector pMMB67EH (Furste, J. P., Pansegrau, W., Frank, R., Blocker, H., Scholz, P., Bagdasarian, M. and Lanka, E. 1986. Gene 48, 119-131). Plasmid pMMB67EH is a derivative of RSF1010, which replicates in most Gram-negative and even some Gram-positive organisms, thus allowing for plasmid-based analysis of sucrose production in E. coli, Synechocystis spp. PCC 6803, Synechococcus elongatus PCC 7942 and a variety of other cyanobacteria (Kreps, S., Ferino, F., Mosrin, C., Gerits, J., Mergeay, M. and Thuriaux, P. 1990. Mol Gen Genet. 221, 129-133; Marraccini, P., Bulteau, S., Cassier-Chauvat, C., Mermet-Bouvier, P. and Chauvat, F. 1993. Plant Molecular Biology 23, 905-909; Gormley, E. P. and Davies, J. 1991. J Bacteriology 173, 6705-8).

The second approach is stable integration into the chromosome of Synechocystis spp. PCC 6803 and Synechococcus elongatus PCC 7942 at the upp (uracil phosphoribosyltransferase) locus. The upp locus was chosen for reasons described below.

### Example 5

### Plasmid-Based Expression

Two plasmids were designed for plasmid-based expression of the asf gene product, pLybAL11 (see e.g., FIG. 6; SEQ ID NO: 19) and pLybAL12 (see e.g., FIG. 7; SEQ ID NO: 20). Plasmid pLybAL12 was constructed for expression from predetermined promoters and pLybAL11 was constructed for expression from promoters selected at random.

Both plasmids were constructed as follows. The asf gene from Synechococcus elongatus PCC 7942 was amplified by PCR with the oligonucleotides 5′-AGACTACAATTGGGGCGTTTTCTGTGAG-3′ (the MfeI restriction endonuclease site is nucleotide positions 7-12) (SEQ ID NO: 7) and 5′-CTTACGTGCCGATCAACGTCTCATTCTGAAAAGGTTAAGCGATCGCCTC-3′ (SEQ ID NO: 8) using whole cells as the template, yielding the product of SEQ ID NO: 1.

The gene encoding for chloramphenicol acetylransferase (cat), both with and without the upstream promoter, was amplified from pBeloBAC11 (GenBank Accession U51113).

The cat gene lacking the promoter was amplified from pBeloBAC11 by PCR with the oligonucleotides 5′-TTATCGCGATCGTCAGGAGCTAAGGAAGCTAAAATGGAG-3′ (SEQ ID NO: 9) and 5′-CGACCAATTCACGTGTTTGACAGCTTATC-3′ (SEQ ID NO: 10) (the PvuI and PmlI restriction endonuclease sites are at nucleotide positions 4-9 and 10-15, respectively) to yield the product of SEQ ID NO: 11.

The cat gene bearing the promoter was amplified from pBeloBAC11 by PCR with the oligonucleotides 5′-TTTTGGCGATCGTGAGACGTTGATCGGCACGTAAG-3′ (SEQ ID NO: 12) and 5′-CGACCAATTCACGTGTTTGACAGCTTATC-3′ (SEQ ID NO: 13) (the PvuI and PmlI restriction endonuclease sites are at nucleotide positions 7-12 and 10-15, respectively) to yield the product of SEQ ID NO: 14.

The PCR products bearing the cat gene were digested with PvuI and the ends blunted with T4 DNA polymerase. They were then individually ligated to the asf PCR product. The resultant products were purified by agarose gel electrophoresis, digested with MfeI and PmlI and then ligated with T4 DNA ligase to the 6.6 Kbp product of pMMB67EH digested with EcoRI and HpaI. The ligation products were transformed into chemically competent NEB5α (New England Biolabs; Ipswich, Mass.) and selected for at 37° C. on LB agar supplemented with 100 μg/ml ampicillin. Selected candidates were grown at 37° C. in LB supplemented with 100 μg/ml ampicillin for miniprep, analyzed by restriction endonuclease digest and then verified by sequence analysis with the oligonucleotides 5′-GCTTCTGCGTTCTGATTTAATCTGTATCAG-3′ (SEQ ID NO: 15), 5′-TATCACTTATTCAGGCGTAGCAACCAG-3′ (SEQ ID NO: 16), 5′-GTCGTTAGTGACATCGACAACACACTG-3′ (SEQ ID NO: 17), and 5′-GATCGCGATACTGATCGAGATAGGTC-3′ (SEQ ID NO: 18). Candidate number 5 of pLybAL11 (pLybAL11-5) (SEQ ID NO: 19) and Candidate number 1 of pLybAL12 (pLybAL12-1) (SEQ ID NO: 20) were chosen for further study.

Based upon plasmid yield during minipreps, it appears that the copy number of these plasmids is greatly reduced when propagated in the E. coli strain NEB Turbo (New England Biolabs; Ipswich, Mass.), suggesting the importance in choice of host strain for these plasmids.

### Example 6

### Promoter Insertion

Six promoters were chosen for insertion into pLybAL12-5. The presumed promoter for Synechocystis spp. PCC 6803 carB encoding carbamoyl phosphate synthase, which is likely to be immediately upstream of the gene pyrR where they would be co-transcribed as an operon, was chosen because it is likely to be strong due to its role in both pyrimidine and arginine biosynthesis. The nitrate reductase (nirA) promoters from both Synechocystis spp. PCC 6803 (Aichi, M., Takatani, N. and Omata, T. 2001. J. Bacteriol. 183, 5840-5847) and Synechococcus elongatus PCC 7942 (Maeda, S-I. et al. 1998. J Bacteriol 180, 4080-4088) were chosen for their ability to be regulated by the source of nitrogen. The strong light-phase promoter for the photosystem II D1 protein (psbAII) from Synechococcus elongatus PCC 7942 (Golden, S. S., Brusslan, J. and Haselkorn, R. 1986. EMBO Journal 5, 2789-2798) and two dark-phase promoters from Synechocystis spp. PCC 6803 [dnaK (Aoki, S., Kondo, T. and Ishiura M. 1995. J Bacteriol 177, 5606-11) and kaiA (Kucho, K−1. et al. 2005. J Bacteriol 187, 2190-2199)] were also selected as regulated cyanobacterial derived promoters. Lastly, the λ_{PR} temperature-regulated promoter, which has been shown to be active in cyanobacteria, was chosen (Ferino, F. and Chauvat, F. 1989. Gene 84, 257-66; Mermet-Bouvier, P. and Chauvat, F. 1994. Current Microbiology 28, 145-148).

The following oligonucleotides were used to amplify the promoters by PCR using whole cells as the template, yielding the products shown. The restriction endonuclease sites incorporated for cloning are provided in the sequence.

Synechocystis spp. PCC 6803 pyrR (SphI/KpnI) (SEQ ID NO: 23) was amplified from whole cells by PCR with the oligonucleotides 5′-CGGTGTGCATGCCGTTATTGATGGAATG-3′ (SEQ ID NO: 21) and 5′-TCACTAGGTACCTAAATTACCTGGGAAGCCAG-3′ (SEQ ID NO: 22), having restriction endonuclease sites at nucleotide positions 7-12 for both.

Synechocystis spp. PCC 6803 nirA (SphI/KpnI) (SEQ ID NO: 26) was amplified from whole cells by PCR with the oligonucleotides 5′-CCCAAGGCATGCAGGAAAACAAGCTCAGAATGCTG-3′ (SEQ ID NO: 24) and 5′-TTTATTGGTACCAACGCTTCAAGCCAGATAACAGTAGAGATC-3′ (SEQ ID NO: 25), having restriction endonuclease sites at nucleotide positions 7-12 for both.

Synechococcus elongatus PCC 7942 psbAII (SphI/KpnI) (SEQ ID NO: 29) was amplified from whole cells by PCR with the oligonucleotides 5′-ATCTTTGCGTTCCGTGACGGCTACTG-3′ (SEQ ID NO: 27) and 5′-GCAGATGGTACCGGTCAGCAGAGTG-3′ (having restriction endonuclease sites at nucleotide positions 7-12) (SEQ ID NO: 28).

Synechococcus elongatus PCC 7942 nirA (SphI/KpnI) (SEQ ID NO: 32) was amplified from whole cells by PCR with the oligonucleotides 5′-CAGCCAGCATGCATAAATTTCTGTTTTGACCAAACCATCC-3′ (SEQ ID NO: 30) and 5′-GTGGCTGGTACCATGGATTCATCTGCCTACAAAG-3′ (SEQ ID NO: 31), having restriction endonuclease sites at nucleotide positions 7-12 for both.

λ_{PR} (XbaI/KpnI) (SEQ ID NO: 35) was amplified from whole cells by PCR with the oligonucleotides 5′-GTGCATTCTAGATGGCTACGAGGGCAGACAGTAAG-3′ (SEQ ID NO: 33) and 5′-TTCTGTGGTACCATATGGATCCTCCTTCTTAAGATGCAACCATTATCACC-3′ (SEQ ID NO: 34), having restriction endonuclease sites at nucleotide positions 7-12 for both.

Synechocystis spp. PCC 6803 dnaK (SphI/KpnI) (SEQ ID NO: 38) was amplified from whole cells by PCR with the oligonucleotides 5′-GCCCCAGCATGCACCAGTAAACATAAATCTC-3′ (SEQ ID NO: 36) and 5′-ATTGGTGGTACCGAGGTCAATCCCAACAAC-3′ (SEQ ID NO: 37), having restriction endonuclease sites at nucleotide positions 7-12 for both.

Synechocystis spp. PCC 6803 kiaA (SphI/KpnI) (SEQ ID NO: 41) was amplified from whole cells by PCR with the oligonucleotides 5′-GCCAGAGCATGCAAAGCTCACTAACTGG-3′ (SEQ ID NO: 39) and 5′-GGAAAAGGTACCTGAGTCTATGGGCAACGTG-3′ (SEQ ID NO: 40), having restriction endonuclease sites at nucleotide positions 7-12 for both.

After amplification, the PCR products were digested with the restriction endonucleases shown above, gel purified, and ligated into similarly digested pLybAL12-1 to yield plasmids pLybAL15 (SEQ ID NO: 44), pLybAL16 (SEQ ID NO: 45), pLybAL17 (SEQ ID NO: 46), pLybAL18 (SEQ ID NO: 47), pLybAL19 (SEQ ID NO: 48), pLybAL21 (SEQ ID NO: 49), and pLybAL21 (SEQ ID NO: 50), respectively. The ligation products were transformed into electrocompetent NEB5a (New England Biolabs; Ipswich, Mass.) and selected for at 30° C. on LB agar supplemented with 100 μg/ml ampicillin, 34 μg/ml chloramphenicol, and 5% sucrose. Selected candidates were grown at 30° C. in LB supplemented with 100 μg/ml ampicillin, 34 μg/ml chloramphenicol and 5% sucrose for miniprep, analyzed by restriction endonuclease digest, and then verified by sequence analysis with the oligonucleotides 5′-GCTTCTGCGTTCTGATTTAATCTGTATCAG-3′ (SEQ ID NO: 42) and 5′-ATGGGTCTGAATGTGCAGAATGTAGAG-3′ (SEQ ID NO: 43). Candidates 6 and 7 (pLybAL15-6 and pLybAL15-7), 2 (pLybAL16-2), 4 and 5 (pLybAL17-4 and pLybAL17-5), 1 and 2 (pLybAL18-1 and pLybAL18-2), 1 and 2 (pLybAL19-1 and pLybAL19-2), 3 and 5 (pLybAL21-3 and pLybAL21-5) and 4 and 8 (pLybAL22-4 and pLybAL22-8) were chosen for plasmids pLybAL15 (SEQ ID NO: 44), pLybAL16 (SEQ ID NO: 45), pLybAL17 (SEQ ID NO: 46), pLybAL18 (SEQ ID NO: 47), pLybAL19 (SEQ ID NO: 48), pLybAL21 (SEQ ID NO: 49), and pLybAL21 (SEQ ID NO: 50), respectively.

Selection and growth of these plasmids on LB supplemented with sucrose and both antibiotics was essential to obtaining clones. Selection was originally conducted on LB supplemented with ampicillin alone, but plasmids containing a promoter could not be isolated. Isolates were either re-ligation of the vector alone or of varying size and lacking the ability to be propagated in the presence chloramphenicol. It is thought that internal sucrose was being produced, creating an osmotic shock for the cells that leads to deletions preventing sucrose production. Subsequent experiments indicated that, once isolated, the plasmids may be stable in the absence of sucrose, possibly through the eventual induction of osmotic stress machinery and/or sucrose consumption enzymes.

### Example 7

### Transformation of Synechocystis and Synechococcus

The promoter-containing plasmids, pLybAL15 (SEQ ID NO: 44), pLybAL16 (SEQ ID NO: 45), pLybAL17 (SEQ ID NO: 46), pLybAL18 (SEQ ID NO: 47), pLybAL19 (SEQ ID NO: 48), pLybAL21 (SEQ ID NO: 49), and pLybAL21 (SEQ ID NO: 50), as well as the promoterless pLybAL12-1 vector (SEQ ID NO: 20) (see Examples 5-6), were placed into both Synechocystis spp. PCC 6803 and Synechococcus elongatus PCC 7942 by triparental conjugation, performed consistent with Elhai, J. and Wolk, C. P. 1988. Methods in Enzymology 167, 747-754, unless indicated otherwise.

Overnight cultures of the cargo strains (NEB5a bearing the plasmids to be transferred), as well as an overnight culture of HB101 bearing the helper plasmid pRK2013 (ATCC 37159) grown at 30° C. were pelleted by centrifugation, washed twice with LB and then resuspended in LB in one-tenth the original volume. Each cyanobacterium was grown at 30° C. in BG11-A, which is the same as BG11 except the trace elements have been replaced with Nitsch's trace elements (Nitsch, J. P. and Nitsch, C. 1956. American Journal of Botany 43, 839-851) under constant illumination to an OD₇₃₀ of approximately 0.5. The cells were pelleted by centrifugation, washed twice with BG11-A, and resuspended in BG11-A with a 7.5-fold increase in concentration. A series of 10-fold dilutions of the cyanobacteria in BG11-A were prepared down to 10⁻⁵. At each dilution, 100 μl of the cyanobacterium was combined with 50 μl each of the cargo and helper strains of E. coli. 150 μl of each mixture was then plated onto BG11-A agar (1.5%) plates supplemented with 5% LB. The plates were incubated at 26-28° C. under constant illumination for 16 to 24 hours. The agar (app. 30 ml) on each plate was lifted and 300 μl of a 100× chloramphenicol solution was added. The final concentration of chloramphenicol was 25 μg/ml for Synechocystis spp. PCC 6803 and 7.5 μg/ml for Synechococcus elongatus PCC 7942. Incubation continued for 8-12 days. Individual colonies of transconjugants were purified away from contaminating E. coli by restreaking onto BG11-A supplemented with the appropriate amount of chloramphenicol to, again, obtain isolated colonies.

### Example 8

### Promoter Library in pLybAL11-5

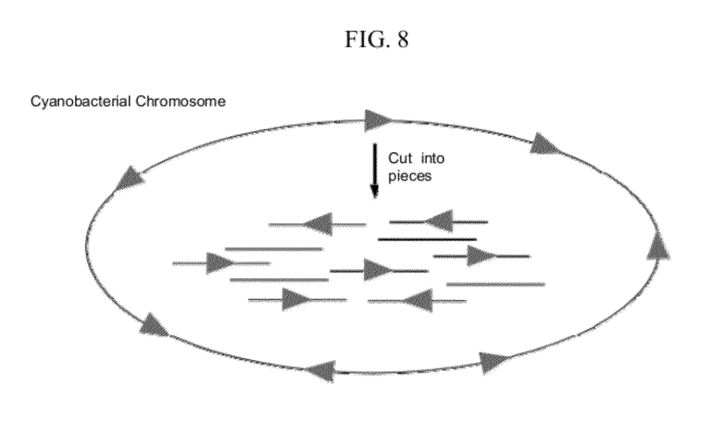

The following example describes construction of a library of cyanobacterial DNA for promoter selection using pLybAL11-5 (SEQ ID NO: 19) (see Example 5). A modified, scaled up version of the chromosomal DNA isolation protocol of Wilson, K. (1997. Preparation of Genomic DNA from Bacteria. In Current Protocols in Molecular Biology. John Wiley and Sons Vol. 1, pp. 2.4.1-2.4.5) was employed, where the primary differences were much longer incubation times and the replacement of SDS with Sarkosyl. The DNA isolated was of sufficient quality for partial Sau3AI digest for insertion into the BamHI site of pLybAL11-5. As shown in FIG. 8, some of the fragments would have promoters and others would not.

During the process of library construction, a possible promoter within the asf gene was discovered. To function as a promoter cloning vector, plasmid pLybAL11-5 (SEQ ID NO: 19) is supposed to only be resistant to chloramphenicol when a promoter has been inserted in front of the asf gene, as the marker lacks its normal promoter and the promoter upstream of asf was not included. Once constructed, however, the chloramphenicol resistance conferred by this plasmid was examined in E. coli. When NEB5a bearing pLybAL11-5 was cultured on LB agar (1.5%) supplemented with 34 μg/ml chloramphenicol at 37° C., growth was observed. When cultured in liquid LB medium supplemented with 34 μg/ml chloramphenicol, however, little-to-no growth was observed. NEB5α bearing pLybAL12-1 (SEQ ID NO: 20) grows in the presence of chloramphenicol on both solid and in liquid LB medium.

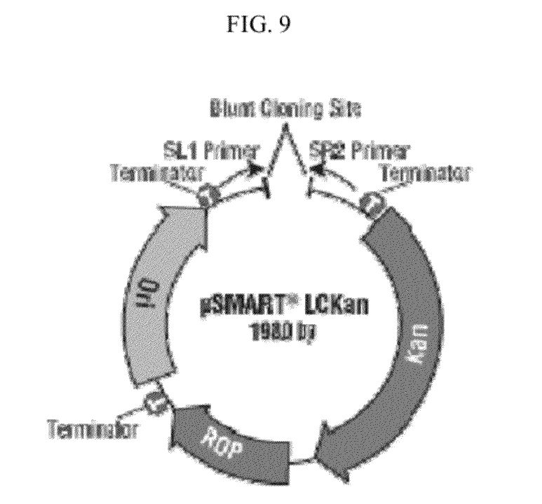

To verify there was no missed promoter upstream of the asf gene but downstream of the transcription terminators, the insert placed into pMMB67EH to make pLybAL11 was cloned into Lucigen Corp.'s (Middleton, Wis.) pSMART-LCKan blunt-end cloning vector using Lucigen's CloneSmart kit with the Lucigen strain of E. coli (E. cloni 10G) competent cells (see e.g., FIG. 9). Because it was blunt-ended cloning, the inserts could ligate to the plasmid in either direction to create pLybAL13f (SEQ ID NO: 51) and pLyAL13r (SEQ ID NO: 52). This vector is specifically designed to eliminate transcription read through from the vector by surrounding the cloning site with terminators. As a control, the insert used to construct pLybAL12 was also placed into this vector, creating pLybAL14f (SEQ ID NO: 53) and pLybAL14r (SEQ ID NO: 54). The plasmids looked to be the appropriate size on an agarose gel but inserts were not verified by DNA sequencing to confirm the integrity of the clones. Similar results, however, were seen for E. cloni 10G bearing pLybAL13 and pLybAL14 (with the cloned DNA ligated in either direction f or r) as were seen for NEB5a bearing pLybAL11 (SEQ ID NO: 19) and pLybAL12 (SEQ ID NO: 20), respectively. This indicates that the activity of this promoter is weak in E. coli.

Many E. coli promoters do not function in cyanobacteria, and vice versa. It is possible that this promoter activity would not be observed in Synechocystis spp. PCC 6803 or Synechococcus elongatus PCC 7942. To check this, pLybAL11-5 (SEQ ID NO: 19) was inserted into both organisms by conjugation, as described above. On BG11-A agar (1.5%) supplemented with chloramphenicol (25 μg/ml and 7.5 μg/mlfor Synechocystis spp. PCC 6803 and Synechococcus elongatus PCC 7942, respectively), growth was observed.

Growth of these organisms bearing pLybAL11-5 (SEQ ID NO: 19) on liquid BG11-A supplemented with chloramphenicol was examined. It is possible that this activity is very weak and is only observable when present on a multiple-copy plasmid. This may be the case with E. coli, but is not likely with the cyanobacteria. RSF1010 is a relatively low-copy plasmid, having only 12 copies in E. coli (Frey, J., Bagdasarian, M. M. and Bagdasarian, M. 1992). Gene 113, 101-106). E. coli undergoing rapid division has at most 2 copies of its chromosome, thus at least a 6-fold increase in copy number. A comparable copy number in cyanobacteria for this plasmid is likely. The chromosomal copy numbers of Synechocystis spp. PCC 6803 and Synechococcus elongatus PCC 7942 of 10-12 and 16, respectively, are similar (Labarre, J., Chauvat, F. and Thuriaux, P. 1989. J Bacteriol 171, 3449-57). The results above suggest the presence of a promoter within the asf gene of cyanobacteria.

FIG. 10 shows a possible location of a promoter (or promoters) within the asf gene. Transcription initiation elements have been described by Curtis, S. E. [1994. The transcription apparatus and the regulation of transcription initiation. In The Molecular Biology of Cyanobacteria. Bryant, D. A. (ed). Kluwer Academic Publishers pp. 613-699]. Translation initiation elements have been defined by Sazuka, T. and Ohara, O. (1996. DNA Research 3, 225-232).

Based upon alignment to known SPS enzymes and the presence of a stop codon only two codons upstream, the translation initiation of the asf gene is predicted to start at a GTG start codon. While ATG start codons are the most common, GTG and TTG are less common, but not rare. A typical E. coli-like Shine-Delgarno sequence (GGAG or GAGG) complementary the 3′-end of the 16S rRNA for which the adenine nucleotide is optimally 9-12 by away from the first nucleotide of the start codon is also present, except with somewhat longer spacing. This sequence is found in about half the genes studied by Sazuka and Ohara. Less optimal spacing is not uncommon, but often leads to reduced levels of expression. There is too little sequence upstream of the Shine-Delgarno sequence but downstream of the MfeI site to incorporate a promoter. It is possible that a partial promoter may be incorporated, but the rest of the promoter would have to produced by the vector sequence of all three plasmids (pLybAL11-5 (SEQ ID NO: 19); pLybAL13f (SEQ ID NO: 51); and pLybAL13r (SEQ ID NO: 52)), which is improbable.

Thus it likely that the promoter activity is located within the asf gene. If the promoter is within the asf gene, one potential position is in front of the SPP domain of asf. This would give the sucrose biosynthetic enzymes of Synechococcus elongatus PCC 7942 a similar quaternary structure to those from Synechocystis spp. PCC 6803. Each organism would have two proteins, an SPS domain with a translationally fused SPP or SPP-like domain and a distinct SPP that may (or may not) interact with each other.

First, it was determined whether the SPP domain of asf could even be translated separately. As can be seen in FIG. 10 and Table 1, there is a TTG start codon immediately upstream of the SPP domain that is preceded by a Shine-Delgarno sequence.

TABLE 1
Nucleotides immediately surrounding the proposed spp start
codon. The nucleotides immediately surrounding the proposed
spp start codon are compared to the consensus of 72 cyano-
bacterial genes. Nucleotides matching the consensus are
italicized, whereas nucleotides that do not match the con-
sensus are underlined. Nucleotide numbers are relative to
the first nucleotide of the start codon.

| NT# | −10 | −9 | −8 | −7 | −6 | −5 | −4 | −3 | −2 | −1 | 123 | 4 | 5 | 6 |
|---|---|---|---|---|---|---|---|---|---|---|---|---|---|---|
| Consensus | A/G | A/G | A/T | A/T | A/T | A/T | A/T | A/T | C/T | T/C | ATG | A/G | C | C/T |
| Selo7942 asf | T | G | A | C | T | A | G | C | G | C | GTG | G | C | A |
| Selo7942 spp | T | C | G | C | A | A | A | C | G | C | TTG | A | T | T |

The region surrounding the start codon matches the consensus determined by Sazuka and Ohara for 72 cyanobacterial genes almost as well as the native start codon. While determining cyanobacterial promoters based upon rules established for E. coli promoters, the typical −35 and −10 elements were searched for since the promoter does appear to be active in E. coli. Two possible promoters were identified, as seen in FIG. 10. There remains the possibility of an additional promoter(s) elsewhere in asf.

### Example 9

### Transfer of Plasmids from E. coli to Cyanobacteria

Conjugation was used for transfer of the pMMB67EH-based plasmids into cyanobacteria. Protocols exist for the transformation of these organisms (Zang, X., Liu, B., Liu, S., Arunakumara, K. K. I. U. and Zhang, X. 2007. Journal of Microbiology 45, 241-245; Golden, S. S, and Sherman, L. A. 1984. Journal of Bacteriology 158, 36-42), but such approaches were unsuccessful for placing these plasmids into Synechocystis spp. PCC 6803 and Synechococcus elongatus PCC 7942 using natural transformation.

The presence of the plasmids in the cyanobacteria was verified. Transconjugants were analyzed for the presence of plasmid by PCR of the asf/cat gene combination with the oligonucleotides 5′-AGACTACAATTGGGGCGTTTTCTGTGAG-3′ (SEQ ID NO: 7) and 5′-GGTGGTTGTGTTTGACAGCTTATC-3′ (SEQ ID NO: 55), yielding a 3.1 kb product. In addition, plasmids were isolated and analyzed. Cultures of cells grown in BG11-A supplemented with chloramphenicol (at the concentrations described above) are pelleted by centrifugation, resuspended in TE, heat-treated and miniprepped by the Promega Wizard SV Plus miniprep kit. But with poor yield, direct plasmid analysis is difficult. As such, the isolated DNA is transformed into E. coli NEB5a, re-isolated using the Promega Wizard SV Plus miniprep kit, and then subjected to restriction endonuclease analysis.

### Example 10

### Sucrose Production Assay and Analysis

Synechococcus transformed with pLybAL19 or pLybAL17 (see Example 7) was assayed for sucrose accumulation. Sucrose is measured with BioVision, Inc.'s (Mountain View, Calif.) sucrose assay kit. Assays were run following a 4 hour induction period (increased light to 180 microeinsteins from 50 microeinsteins for pLybAL17 (SEQ ID NO: 46) and increased temperature from 26 to 39° C. for pLybAL19 (SEQ ID NO: 48)). Data was corrected for background glucose present in the cells.

Results showed Synechococcus transformed with pLybAL19 (SEQ ID NO: 48) accumulated 0.78 nanomoles of sucrose per mg of dry biomass. Results also showed that Synechococcus transformed with pLybAL17 (SEQ ID NO: 46) accumulated 0.95 nanomoles of sucrose per mg of dry biomass.

Further analysis for plasmid-based sucrose production in E. coli, Synechocystis spp. PCC 6803, and Synechococcus elongatus PCC 7942 was performed. Because bacteria can consume sucrose, detection may be difficult. As such, cells are grown under suppressing conditions and then assayed shortly after induction. The pyrR promoter may be suppressed by growth with uracil and induced by transfer medium lacking uracil. The nirA promoters can be suppressed by growth with ammonium ions as the nitrogen source and induced by transfer to medium with nitrate as the nitrogen source. The psbAII promoter can be shifted from low light to high light. The dark phase promoters can be shifted from light to dark. And, the λ_{PR} promoter can be shifted from low (25° C.) to high (39° C.) temperature.

### Example 11

### Expression Through Stable Chromosomal Integration

Insertion of sucrose biosynthetic genes can cause a negative impact on cell growth, leading to difficulties in obtaining complete segregation of the 10-16 chromosomes. With normal selection for an antibiotic resistance marker, having additional copies of the marker does not dramatically impact the cells ability to survive in the presence of antibiotic. Therefore, complete chromosomal segregation can be difficult to achieve using antibiotic selection when faced with a negative phenotype.

Deletion of the upp gene (encoding for uracil phosphoribosyltransferase) in most organisms leads to resistance to the otherwise toxic 5-fluorouracil. To obtain complete resistance, all copies of the upp gene must be deleted. Thus integrating into the upp locus of Synechocystis spp. PCC 6803 (SEQ ID NO: 56) and Synechococcus elongatus PCC 7942 (SEQ ID NO: 58) will lead to 5-fluorouracil resistance and allow for positive selection of complete segregation, even in the presence of a negative phenotype.

### Example 12

### The UPP/Kanamycin Resistance Cassette

A general strategy for genomic manipulation using a upp/kanamycin resistance cassette is outlined in FIG. 11. Deletion of a gene is depicted, but the strategy can easily be modified at the “replacement” step for insertions and mutations.

An upp/kanamycin resistance cassette was constructed. The cassette was constructed in Epicentre Biotechnologies CopyControl cloning kit with blunt-end cloning vector pCC1 and E. coli strain EPI300 according to manufacturer protocols. The upp gene from Bacillus subtilis 168 was amplified from whole cells using the oligonucleotides 5′-AAGAAGCAAGACAGCGTGTAGCTGCTCTGACTG-3′ (SEQ ID NO: 60) and 5′-TCCCGGGATTTGGTACCTTATTTTGTTCCAAACATGCGGTCACCCGCATC-3′ (having restriction endonuclease sites at nucleotide positions 2-7 and 12-17) (SEQ ID NO: 61), yielding the product of SEQ ID NO: 62.

The PCR product was cloned into pCC1 and those bearing the insert were selected for on LB supplemented with chloramphenicol as described in Epicentre Biotechnologies' protocol. The forward orientation, relative to lacZ, was screened for by restriction endonuclease digest, yielding pLybAL7f (SEQ ID NO: 65). The exact sequence of the insert was verified by DNA sequencing with the oligonucleotides 5′-GTAATACGACTCACTATAGGGC-3′ (SEQ ID NO: 63) and 5′-CACACAGGAAACAGCTATGACCAT-3′(SEQ ID NO: 64) for candidates 3 and 8 (pLybAL7-3 and pLybAL7-8).

The kanamycin resistance marker from the Lybradyn vector pLybAA1 [originally derived from pACYC177 (Rose, R. E. 1988. Nucleic Acids Res. 16, 356] was amplified with the oligonucleotides 5′-GTCAGTGCACTGCTCTGCCAGTGTTACAACC-3′ (having ApaLI restriction endonuclease sites at nucleotide positions 5-10) (SEQ ID NO: 66) and 5′-CTCAGTGGCGCCAAAACTCACGTTAAGGGATTTTGGTC-3′ (SEQ ID NO: 67) (having Nan restriction endonuclease sites at nucleotide positions 7-12), yielding the product of SEQ ID NO: 68.

The PCR product was digested with ApaLI and Nan and ligated into similarly digested pLybAL7f, creating pLybAL8f (SEQ ID NO: 69). The proper plasmid was selected for on LB supplemented with 50 μg/ml neomycin and examined by restriction endonuclease digestion.

### Example 13

### UPP Deletion

One strategy to force segregation of chromosomal inserts for the expression of sugars, including sucrose, trehalose, glucosylglycerol, and mannosylfructose, utilizes deletion of upp from the chromosome leading to resistance to 5-fluorouracil. While this has been established in many organisms (such as E. coli and B. subtilis), it has not previously been established for cyanobacteria, such as Synechocystis spp. PCC 6803 and Synechococcus elongatus PCC 7942.

Testing showed that growth of each of these organisms was completely inhibited by 1 μg/ml, 5-fluorouracil. Growth of Synechocystis spp. PCC 6803 is completely inhibited by 0.5 μg/ml, 5-fluorouracil and is sensitive to as little as little as 0.1 μg/ml, 5-fluorouracil.

The upp gene and surrounding sequences of both Synechocystis spp. PCC 6803 was amplified with the oligonucleotides Sspupp-F (SEQ ID NO: 96) and Sspupp-R (SEQ ID NO: 97). The upp gene and surrounding sequences of Synechococcus elongatus PCC 7942 was amplified with the oligonucleotides Seloupp-F (SEQ ID NO: 98) and Seloupp-R (SEQ ID NO: 99). The PCR products (upp of Synechocystis spp. PCC 6803, SEQ ID NO: 100; upp of Synechococcus elongatus PCC 7942, SEQ ID NO: 101) were then cloned into the Epicentre Biotechnologies' (Madison, Wis.) blunt cloning vector pCC1, as per the manufacturer's instructions.

While the PCR product (SEQ ID NO: 100 or SEQ ID NO: 101) can ligate into pCC1 in either direction, the forward orientation relative to the lac promoter was chosen, generating pLybAL3f (SEQ ID NO: 102) (containing upp of Synechocystis spp. PCC 6803) and pLybAL5f (SEQ ID NO: 103) (containing upp of Synechococcus elongatus PCC 7942), respectively. The inserts were sequenced using oligonucleotides T7Iong (SEQ ID NO: 104) and M13rev (SEQ ID NO: 105). The nucleotide sequence of upp of Synechocystis spp. PCC 6803 is represented by SEQ ID NO: 111 and the polypeptide sequence by SEQ ID NO: 112. The nucleotide sequence of upp of Synechococcus elongatus PCC 7942 is represented by SEQ ID NO: 113 and the polypeptide sequence by SEQ ID NO: 114.

Plasmid pLybAL4f (SEQ ID NO: 106) was created from pLybAL3f (SEQ ID NO: 102) by removal of the BlpI and ApaLI fragment, blunt ending with T4 DNA polymerase and then recircularizing with T4 DNA ligase. Part of the Synechocystis spp. PCC 6803 upp gene was then deleted by digesting pLybAL4f with AvrII and SgfI, blunt ending with T4 DNA polymerase and then recircularizing with T4 DNA ligase, creating pLybAL9f (SEQ ID NO: 107). The SacI/SphI fragment (SEQ ID NO: 108) bearing the cyanobacterial DNA was excised from pLybAL9f (SEQ ID NO: 107) and ligated into similarly digested pARO180 (sequence not completely known; Parke, D. 1990. Construction of mobilizable vectors derived from plasmids RP4, pUC18 and pUC19. Gene 93:135-137; ATCC 77123), creating pLybAL25. Plasmid pLybAL6fb (SEQ ID NO: 109) was created from pLybAL5f by removal of the SapI and ApaLI fragment, blunt ending with T4 DNA polymerase and then recircularizing with T4 DNA ligase. Part of the Synechococcus elongatus PCC 7942 upp gene was then deleted by digesting pLybAL6fb with BssHII and BsaI, blunt ending with T4 DNA polymerase and then recircularizing with T4 DNA ligase, creating pLybAL10fb (SEQ ID NO: 110). The SacI/SphI fragment (SEQ ID NO: 138) bearing the cyanobacterial DNA was excised from pLybAL10fb and ligated into similarly digested pARO180, creating pLybAL26.

Plasmids pLybAL25 and pLybAL26 were placed in E. coli S17-1 (ATCC 47055). Plasmids pLybAL25 and pLybAL26 are to be transferred to Synechocystis spp. PCC 6803 and Synechococcus elongatus PCC 7942 by biparental conjugation. Since these plasmids do not replicate in cyanobacteria, they should function as suicide vectors and cross over into the chromosome, deleting upp on one of the copies of the chromosome. An optimized protocol will enable speeding of segregation without killing the cells by premature exposure to too much 5-fluorouracil.

### Example 14

### Modification of Sucrose Degradation Enzymes

Cyanobacteria transformed with asf are further engineered to improve sucrose production by modulation of sucrose degradation activity.

The inventors have identified genes encoding invertase homologues in both Synechocystis spp. PCC 6803 (nucleotide sequence SEQ ID NO: 70; polypeptide sequence SEQ ID NO: 71) and Synechococcus elongatus PCC 7942 (nucleotide sequence SEQ ID NO: 72; polypeptide sequence SEQ ID NO: 73). Synechocystis spp. PCC 6803 also encodes a sucraseferredoxin-like protein (nucleotide sequence SEQ ID NO: 74; polypeptide sequence SEQ ID NO: 75) (Machray G. C. et al. 1994. FEBS Lett 354, 123-127).

These genes are deleted using the markerless deletion protocol described in FIG. 11.

### Example 15

### Modification of Sucrose Degradation Enzymes

Cyanobacteria transformed with asf are further engineered to promote sucrose secretion from the cells.

When in a low osmotic environment, the sucrose may be automatically expunged from the cells, as done with osmoprotectants by some organisms when transitioning from high to low salt environments (Schleyer, M., Schmidt, R. and Bakker, E. P. 1993. Arch Microbiol 160, 424-43; Koo, S. P., Higgins, C. F. and Booth, I. R. 1991. J Gen Microbiol 137, 2617-2625; Lamark, T., Styrvold, O. B. and Strgim, A. R. 1992. FEMS Microbiol. Lett 96, 149-154). Engineering of cyanobacteria can promote such a process.

Cyanobacteria transformed with asf are further engineered to express sucrose permease, such as those used by E. coli and Salmonella or in the transport of sucrose to nitrogen-fixing cysts of certain cyanobacteria (Jahreis K. et al. 2002. J Bacteriol 184, 5307-5316; Cumino, A. C. 2007. Plant Physiol 143, 1385-97). These genes are cloned and transformed into cyanobacteria according to techniques described above.

### Example 16

### Sucrose Secretion by Cyanobacteria Transformed with Porin

Sucrose secretion from Synechocystis spp. PCC 6803 and Synechococcus elongatus PCC 7942 can be facilitated by transformation with sucrose porin.

The gene encoding sucrose porin (scrY) from Enterobacter sakazakii ATCC BAA-894 was cloned for expression in Synechocystis spp. PCC 6803 and Synechococcus elongatus PCC 7942. The function of this gene has been inferred from its sequence and those of its neighbors. Enterobacter sakazakii scrY was amplified from chromosomal DNA by PCR with the oligonucleotides EsscrYBamHI-F (SEQ ID NO: 88) and EsscrYSacI-R (SEQ ID NO: 89). The PCR product (SEQ ID NO: 90) was digested with BamHI and SacI and ligated into similarly digested pLybAL19 and cloned into NEB5α, creating pLybAL32 (SEQ ID NO: 91). The scrY gene (nucleic acid SEQ ID NO: 94; polypeptide sequence, SEQ ID NO: 95) was then sequenced with the oligonucleotides EsscrYmidseq-F (SEQ ID NO: 92) and EsscrYmidseq-R (SEQ ID NO: 93). When introduced into the host, this construct allows for the co-expression of the genes scrY and asf under the control of the temperature-inducible promoter. This plasmid was transferred by tri-parental conjugation (as described above) into Synechocystis spp. PCC 6803. The transformed Synechocystis spp. PCC 6803 is tested for efficacy in the secretion of sucrose. Similar transformation and testing of Synechococcus elongatus PCC 7942 follows.

### Example 17

### Generation of Trehalose Accumulating Cyanobacteria

The trehalose biosynthetic genes encoding trehalose phosphate synthase and trehalose phosphate phosphatase (otsA and otsB, respectively) from E. coli are found in a two gene operon, otsBA (SEQ ID NO: 115). The operon was cloned by PCR amplification of E. coli K₁₂ genomic DNA with the oligonucleotides EcotsBA-F (SEQ ID NO: 116) and EcotsBA-R (SEQ ID NO: 117). The PCR product was digested with AfIII and NheI and was cloned into pLybAL19 (SEQ ID NO: 48), replacing most of the asf gene. The new plasmid, pLybAL23 (SEQ ID NO: 118), places the trehalose biosynthetic genes under the control of the temperature-inducible λ_{PR} promoter. The genes were sequenced to verify their integrity with the oligonucleotides EcotsBAmidseq-F (SEQ ID NO: 119) and EcotsBAmidseq-R (SEQ ID NO: 120). Expression of the otsBA operon was then placed under control of the pyrR, psbAII, dnaK and kiaA promoters (as described above) by ligating the AfIII (blunt-ended with T4 DNA polymerase)/NheI fragment of pLybAL23 bearing the otsBA operon, into pLybAL15, pLybAL17, pLybAL21 and pLybAL22 digested with SacI (blunt-ended with T4 DNA polymerase) and NheI, creating pLybAL28 (SEQ ID NO: 121), pLybAL29 (SEQ ID NO: 122), pLybAL30 (SEQ ID NO: 123), and pLybAL31 (SEQ ID NO: 124), respectively.

Each of plasmids pLybAL28 (SEQ ID NO: 121), pLybAL29 (SEQ ID NO: 122), pLybAL30 (SEQ ID NO: 123), and pLybAL31 (SEQ ID NO: 124) were moved into Synechocystis spp. PCC 6803 by tri-parental conjugation (as described above).

Expression of the otsBA operon from pLybAL23 was placed under the control of the Synechocystis spp. PCC 6803 and Synechococcus elongatus PCC 7942 nirA promoters (as described above) in pLybAL16 and pLybAL18 in the same way as just described for the other promoters, creating pLybAL36 (SEQ ID NO: 125) and pLybAL37 (SEQ ID NO: 126), respectively.

### Example 18

### Trehalose Assay

Biomass was separated from the culture broth as necessary by centrifugation and residual biomass was removed from the clarified culture broth by filtration through 0.2 micron filter. The culture broth was concentrated to a residue by evaporation under reduced pressure. The concentrated culture broth was dissolved in 1 ml of de-ionized water and then 10 microliters of solution was sampled in a trehalose assay. The biomass collected by centrifugation was transferred to a weigh dish and heated to 100° C. to remove residual moisture. The dry biomass was weighed and then a 100 mg sample was dissolved in 1 ml of de-ionized water. The mixture was then ground and the solids were removed by centrifugation. A 10 microliter sample of the clarified supernatant was diluted 100 fold with de-ionized water and 10 microliters of the diluted sample were tested for trehalose.

The assay for trehalose used a modified procedure of a commercially supplied sucrose assay kit available through Biovision, Inc. The modification to the standard protocol was the substitution of trehalase for the kit supplied invertase enzyme solution. The kit involves the hydrolysis of trehalose with trehalase to release glucose. The glucose is oxidized by glucose oxidase to produce hydrogen peroxide which is detected by the action of peroxidase in the presence of a colored indicator. The colored indicator is quantitatively measured by its characteristic absorbance at 570 nm to afford the concentration of glucose originally present in the sample.

Trehalase (treA nucleic acid SEQ ID NO: 134 encoding trehalase polypeptide SEQ ID NO: 135) was prepared from the recombinant E. coli treA gene which has been engineered into a plasmid and transformed into an E. coli host by a similar method as described by Gutierrez C, Ardourel M, Bremer E, Middendorf A, Boos W, Ehmann U. Mol Gen Genet. 1989 June; 217(2-3):347-54. Periplasmic trehalase was cloned from E. coli K₁₂, encoded by treA. The treA PCR product (SEQ ID NO: 127) was digested with AfII/XbaI and then ligated into similarly digested pLybCB6, a proprietary plasmid with a constitutive version of the strong E. coli trp promoter, creating pLybAL24 (SEQ ID NO: 130). The integrity of the insert was analyzed by sequencing with the oligonucleotides EctreAmidseq-F and EctreAmidseq-R.

A C-terminal His₆-tagged version of the trehalase was constructed. The gene was amplified by PCR with the oligonucleotides EctreA-F2 (SEQ ID NO: 131) and EctreA-R2 (SEQ ID NO: 132). The PCR product (SEQ ID NO: 136) was then digested with AfII/XbaI and then ligated into similarly digested pLybAL24, creating pLybAL33 (SEQ ID NO: 133).

Strong constitutive expression of the periplasmic trehalase is detrimental to the cells, causing a strong growth defect at 37° C. This can be significantly alleviated by growing the cells at 30° C.

The protein was expressed in E. coli BW25113 on a plasmid pLYBAL24 (SEQ ID NO: 130) which was grown in 2xYT media containing kanamycin. The protein was produced constitutively using the Trp promoter and contains a signal peptide which allows the protein to be transported to the periplasm. Following fermentation and harvesting of the biomass, the enzyme was purified by selective periplasmic release by treatment of the washed and resuspended cell pellet with 2% v/v dichloromethane in 50 mM Tris buffer pH 8. The lysate was separated from cell debris by centrifugation and further processed by concentration using an Amicon ultrafilter fitted with a 10,000 Dalton membrane. The concentrated lysate may be used in assays directly or the enzyme can be further purified by metal ion affinity chromatography using the engineered 6× poly histidine tag on the C-terminus of the enzyme (SEQ ID NO: 137).

### Example 19

### Solid Phase Trehalose Production

A solid composite fabric covered hydrophilic foam composed of a substrate foam used as a media/moisture reservoir (Foamex Aquazone) was bound to a fabric layer (DuPont Sontara) used as a growth surface measuring 15 cm by 15 cm. The composite material was sterilized by soaking in 70% ethanol in water and then hung in a vertical bioreactor plumbed to deliver solutions to the top of the composite material. The solutions were allowed to percolate through the growing composite surface by gravity. Residual ethanol was removed from the sterilized growing surface by passage of 1 liter of sterile de-ionized water flowing at 0.2 ml/min. The growing surface was equilibrated with culture media by flowing 0.5 liters of BG11A growth medium containing 10 micrograms/ml chloramphenicol through the composite material at 0.2 ml/min.

The equilibrated, sterile growth surface was inoculated by flooding the surface with 10 ml of a 4 day pre-culture of Synechocystis spp. PCC 6803 transformed by plasmid pLYBAL23. Following 30 minute incubation the reactor was turned to a vertical position and the fermentation was begun. The reactor was illuminated with 80 microeisteins of light from a white LED array. Temperature was maintained at 28° C., by a resistive heating device attached to the bioreactor. The reactor was continuously purged with 0.2 micron filtered air at 0.2 L/min and fresh culture media was supplied by pump and gravity percolation through the foam layer of the growth composite at a rate of 0.2 ml/min for 30 minutes every 6 hours. The reactor was run continuously for 4-7 days during which the growth surface of the composite was overspread with a dense lawn of cyanobacteria. Following the initial cultivation period the temperature of the bioreactor was increased to 40° C. and maintained at this temperature for an additional 24 hours. During the elevated temperature period spent culture broth was collected and processed for trehalose determination. At the completion of the fermentation run the biomass was collected by rinsing the growth surface with de-ionized water which can be processed for trehalose assay.

The amount of trehalose produced and retained in the biomass grown on the solid surface was up to 2.5 wt % of the total dry weight biomass recovered from the bioreactor following temperature induction. 0.8 wt % of the dry biomass equivalent weight of trehalose was recovered from the culture medium following temperature induction.

### Example 20

### Trehalose Production Liquid Phase

1 liter of sterile BG11A media was prepared in a Bioflow reactor to which chloramphenicol was added to a concentration of 10 micrograms/ml. The reactor was then inoculated with a 5% by volume, 4 day pre-culture of Synechocystis spp. PCC 6803 transformed with plasmid pLYBAL23. The reactor was run at 28° C., 300 RPM, 0.2 L/min 0.2 micron filtered air purge and illuminated at 80 microeinsteins of light using a fluorescent bulb array. The cultivation was maintained for 4-7 days following which a 200 ml sample was removed for processing and trehalose assay. The temperature of the fermentation was then elevated to 40° C. for 24 hours. A 200 ml sample was then removed from the bioreactor for processing and trehalose assay.

Following temperature induction the dried biomass produced up to 3 wt % trehalose while the spent culture broth contained 0.3 wt % trehalose equivalent relative to biomass.

1. A method for processing a feedstock comprising:
cultivating a photosynthetic microorganism;
isolating a feedstock from the photosynthetic microorganism; and
processing the feedstock;
wherein at least one of the following is satisfied
(i) cultivating the photosynthetic microorganism comprises cultivating the photosynthetic microorganism in a photobioreactor, the photobioreactor comprising (a) a non-gelatinous, solid cultivation support suitable for providing nutrients and moisture to photosynthetic microorganisms on at least a portion of a surface thereof, wherein said portion of the surface has a topography that allows photosynthetic microorganisms to adhere thereto when said portion of the surface is oriented non-horizontally; and (b) a physical barrier covering at least said portion of the surface of the cultivation support, wherein the physical barrier is configured so as to allow inoculation of said portion of the surface of the cultivation support, formation and maintenance of an environment suitable for the cultivation of such photosynthetic microorganisms, and harvesting of such cultivated photosynthetic microorganisms; or
(ii) the photosynthetic microorganism is a transgenic photosynthetic microorganism comprising an artificial DNA construct comprising, as operably associated components in the 5′ to 3′ direction of transcription: (a) a promoter functional in the photosynthetic microorganism cell; (b) (1) a polynucleotide comprising a first nucleotide sequence encoding a polypeptide having disaccharide phosphate synthase activity and a second nucleotide sequence encoding a polypeptide having disaccharide phosphate phosphatase activity or (2) a polynucleotide comprising a nucleotide sequence encoding a polypeptide having disaccharide phosphate synthase activity and disaccharide phosphate phosphatase activity; and (c) a transcriptional termination sequence; wherein the transgenic photosynthetic microorganism accumulates increased levels of the disaccharide compared to a photosynthetic microorganism not comprising the DNA construct.

2. The method of claim 1, wherein the feedstock comprises a biomass and processing the feedstock comprises treating the biomass with one or more biomass degrading enzymes derived from a non-vascular photosynthetic organism for a sufficient amount of time to degrade at least a portion of said biomass to form a biofuel, wherein, optionally, said biomass degrading enzyme is a cellulolytic enzyme, a hemicellulolytic enzyme, or a ligninolytic enzyme.

3. The method of claim 1, processing the feedstock comprises contacting under catalytic cracking conditions the feedstock with a catalytic composition thereby making a fuel product.

4. The method of claim 1, wherein cultivating the photosynthetic microorganism comprises contacting the photosynthetic microorganism with a flue gas; and isolating the feedstock comprises extracting a fuel product from the photosynthetic microorganism.

5. The method of claim 1, wherein the feedstock comprises a first fuel product; and cultivating the photosynthetic microorganism comprises contacting the photosynthetic microorganism with a source of inorganic carbons comprising ^{Δ13}C thereby incorporating carbons from the source of inorganic carbons into the first fuel product.

6. The method of claim 1, comprising obtaining a measurement of a ^{Δ13}C distribution of the processed feedstock; comparing the ^{Δ13}C distribution of the processed feedstock to a reference ^{Δ13}C distribution; selling carbon credits to an entity if the ^{Δ13}C distribution of the processed feedstock is less than the reference ^{Δ13}C distribution, wherein the entity is an owner or user of the processed feedstock.

7. The method of claim 1, wherein the processed feedstock comprises a food composition comprising at least 0.1% w/w biomass of the photosynthetic microorganism and one or more other edible ingredients, wherein the biomass comprises at least 10% oil by dry weight.

8. The method of claim 1, wherein the feedstock comprises a triglyceride oil and processing the feedstock comprises subjecting the triglyceride oil to one or more chemical reactions to generate alkanes, whereby fuel is produced, and, optionally, wherein the one or more chemical reactions is a chemical reaction selected from the group consisting of transesterification, hydrogenation, hydrocracking, deoxygenation, isomerization, interesterification, hydroxylation, hydrolysis to yield free fatty acids, and saponification.

9. The method of claim 1, wherein the feedstock comprises a saccharide and processing the feedstock comprises providing the saccharide as a carbon source.

10. The method of claim 1, wherein cultivating the photosynthetic microorganism comprises cultivating the photosynthetic microorganism in the presence of a fixed carbon source, wherein the photosynthetic microorganism contain an exogenous gene; the photosynthetic microorganism accumulate at least 10% of their dry cell weight as lipid; and the fixed carbon source is selected from the group consisting of sorghum and depolymerized cellulosic material; and isolating lipid components from the cultured microorganisms.

11. The method of claim 1, wherein the feedstock comprises a saponified microbial lipid and the processed feedstock comprises a soap comprising the saponified microbial lipid.

12. The method of claim 1, wherein the feedstock comprises lipids at least 5% of the photosynthetic microorganism dry weight and processing the feedstock comprises saponifying the lipid to obtain a soap.

13. The method of claim 1, wherein the processed feedstock is used to treat mammalian skin or stimulate collagen synthesis in mammalian skin, wherein treating comprises applying, injecting, or orally administering the processed feedstock to a mammalian subject.

14. The method of claim 1, wherein the processed feedstock comprises a polysaccharide and hyaluronic acid.

15. The method of claim 1, wherein the processed feedstock comprises a polysaccharide and the processed feedstock is used to lubricate the joint of a mammal by injecting the processed feedstock into a cavity containing synovial fluid of the mammal.

16. The method of claim 1, wherein cultivating the photosynthetic microorganism comprises cultivating the photosynthetic microorganism on glycerol as a source of fixed carbon to produce the feedstock; processing the feedstock comprises separating lipid from aqueous components of the feedstock and, optionally, deoxygenating the lipid, hydrogenating the lip, or hydrocracking the lipid.

17. The method of claim 1, wherein
cultivating the photosynthetic microorganism comprises cultivating the photosynthetic microorganism in the presence of a fixed carbon source, wherein the photosynthetic microorganisms accumulate at least 10% of their dry cell weight as lipid; and the fixed carbon source is selected from the group consisting of glycerol, depolymerized cellulosic material, sucrose, molasses, glucose, arabinose, galactose, xylose, fructose, arabinose, mannose, acetate, and any combination of the foregoing;
isolating the feedstock comprises isolating lipid components from the photosynthetic microorganisms; and
processing the feedstock comprises subjecting the lipid components to one or more chemical reactions to generate straight chain alkanes to form renewable diesel.

18. The method of claim 1, wherein the photosynthetic microorganisms are capable of heterotrophic growth; and cultivating the photosynthetic microorganisms comprises cultivating the photosynthetic microorganisms in a culture medium comprising a processed feedstock comprising a disaccharide.

19. The method of claim 1, wherein isolating the feedstock comprises recovering lipid from biomass produced by the photosynthetic microorganisms and processing the feedstock comprises subjecting the lipid to transesterification to produce fatty acid ester(s) and glycerol; and the processing the feedstock further comprises adding the glycerol to a microbial culture to form a biodiesel.

20. The method of claim 1, wherein processing the feedstock comprises disrupting the photosynthetic microorganisms to form a homogenate nutraceutical composition.

21. The method of claim 1, wherein the processed feedstock comprises a polysaccharide and a biologically acceptable carrier that is administered to a subject to lower serum cholesterol.

22. The method of claim 1, wherein the processed feedstock comprises a polysaccharide and the processed feedstock is packaged in a sexually transmitted disease prevention kit or composition further comprising a prophylactic device.

23. The method of claim 1, wherein isolating the feedstock comprises adding isopropanol to the cultivated photosynthetic microorganisms and separating precipitated exopolysaccharide.

24. The method of claim 1, wherein the photobioreactor comprises at least one of the following features:
(i) photosynthetic microorganisms are cultivated at a density of at least about 50 grams of dry biomass per liter equivalent;
(ii) the cultivation support comprises a flexible material;
(iii) the cultivation support comprises one or more rigid materials;
(iv) the cultivation support comprises at least two layers, a first layer adjacent to a second layer, the first layer comprising a high surface area growth material and the second layer comprises a permeable type material;
(v) the cultivation support comprises flexibly connected rigid portions, wherein the rigid portions are comprised of the one or more rigid materials;
(vi) the physical barrier it is at least substantially impermeable to solid particulate and liquid but does not prevent the transport of gas or vapor to and from the space proximate to said portion of the surface of the cultivation support nor actinic irradiation of said portion of the surface of the cultivation support;
(vii) the photobioreactor comprises a source of actinic radiation situated to provide photosynthetically active radiation to at least a portion of the cultivation support;
(ix) the physical barrier is between the cultivation support and a source of actinic radiation and is sufficiently transparent to such actinic radiation and sufficiently gas permeable to allow for photosynthesis by the photosynthetic microorganisms during cultivation;
(x) the physical barrier is sufficiently impermeable to water vapor so that the cultivation support upon being moistened will retain enough of the moisture so the photosynthetic microorganisms remain adequately hydrated during cultivation;
(xi) the physical barrier is configured to enclose the cultivation support and any photosynthetic microorganisms thereon, and to be releasably sealed during at least a portion of the cultivation of the photosynthetic microorganisms;
(xii) the photobioreactor comprises a single cultivation support;
(xiii) the photobioreactor comprises a plurality of cultivation supports;
(xiv) the physical barrier is flexible;
(xv) the physical barrier further comprises a first portion that is at least substantially impermeable to solid particulate, liquid, gas, and vapor, and a second portion that is permeable to gas and vapor but at least substantially impermeable to solid particulate and liquid;
(xvi) the physical barrier further comprises a first portion that is at least substantially impermeable to solid particulate, liquid, gas, and vapor, and a second portion that is permeable to gas and vapor but at least substantially impermeable to solid particulate and liquid, the second portion of the barrier has a gas or vapor exchange rate that is from at least about 5 Gurley seconds to no greater than about 10,000 Gurley seconds;
(xvii) the physical barrier further comprises a first portion that is at least substantially impermeable to solid particulate, liquid, gas, and vapor, and a second portion that is permeable to gas and vapor but at least substantially impermeable to solid particulate and liquid, the second portion of the barrier comprises a selective membrane comprising olefin fiber or polyethylene fiber material, polytetrafluoroethylene filtration media, cellulosic filter material, fiberglass filter material, polyester filter material, polyacrylate filter material, polysulfone membranes, or nylon membranes.
(xviii) the physical barrier further comprises a first portion that is at least substantially impermeable to solid particulate, liquid, gas, and vapor, and a second portion that is permeable to gas and vapor but at least substantially impermeable to solid particulate and liquid, the first portion is at least substantially transparent to actinic radiation and the second portion is not at least substantially transparent to actinic radiation, and the configuration of the first and second portions relative to each other and at least said portion of the surface of the cultivation support is such that there a sufficient amount of actinic radiation and gas exchange to support photosynthesis by photosynthetic microorganisms;
(xix) the cultivation support comprises a fabric;
(xx) the cultivation support comprises a fabric, the fabric comprising fibers that are natural, modified natural, synthetic, or a combination thereof;
(xxi) the cultivation support comprises a fabric, the fabric comprising a woven fabric, a knitted fabric, a felt, a mesh of cross-linked fiber polymers, or a combination thereof;
(xxii) the cultivation support comprises a natural fiber selected from the group consisting of cotton, wool, hemp, tree fiber, other cellulosic fibers, and combinations thereof;
(xxiii) the cultivation support comprises a modified natural fiber selected from the group consisting of nitrocellulose, cellulose acetate, cellulose sulfonate, crosslinked starches, and combinations thereof, and a synthetic fiber selected from the group consisting of polyester, polyacrylate, polyamine, polyamide, polysulfone, and combinations thereof;
(xxiv) the cultivation support is coated with a moisture absorbent polymer;
(xxv) the fabric, the fiber of the fabric, or both, are coated with a moisture absorbent polymer;
(xxvi) the moisture absorbent polymer is selected from the group consisting of agar, polyacrylate, polyamide, polyamine, polyethylene glycol, modified starches, and combinations thereof;
(xxvii) the photobioreactor comprises water, nutrients, or a combination thereof on, within, or on and within, the cultivation support;
(xxviii) the photobioreactor comprises one or more attachment points for attaching the photobioreactor to a structure;
(xxix) the solid cultivation support further comprises one or more attachment points for attaching the cultivation support;
(xxx) the photobioreactor comprises at least one of a fluid supply system, a nutrient supply system, a gas supply system, and a microorganism supply system;
(xxxi) at least a portion of the photosynthetic microorganisms are adhered to the solid cultivation support;
(xxxii) the photobioreactor comprises a plurality of solid cultivation supports radiating outward from a central point;
(xxxiii) the photobioreactor comprises a conveyance system that moves a plurality of solid cultivation supports radiating outward from a central point so as to optimize position of one or more solid cultivation supports for receiving light;
(xxxiv) the physical barrier comprises at least a portion sufficiently transparent to actinic radiation and the position of the portion is movable to optimize receipt of light by the solid cultivation support; and
(xxxv) the solid cultivation support comprises a material having loops or is terry cloth.

25. The method of claim 21, wherein the photosynthetic microorganism comprises one or more of:
(a) a polynucleotide comprising a nucleotide sequence encoding a polypeptide selected from the group consisting of:
SEQ ID NO: 2 or a sequence 95% identical thereto having sucrose phosphate synthase and sucrose phosphate phosphatase (ASF) activity;
SEQ ID NO: 4 or a sequence 95% identical thereto having sucrose phosphate synthase (SPS) activity;
SEQ ID NO: 6 or a sequence 95% identical thereto having a sucrose phosphate phosphatase (SPP) activity;
SEQ ID NO: 77 or a sequence 95% identical thereto having trehalose phosphate synthase (TPS) activity;
SEQ ID NO: 79 or a sequence 95% identical thereto having trehalose phosphate phosphatase (TPP) activity;
SEQ ID NO: 81 or a sequence 95% identical thereto having glucosylglycerol phosphate synthase (GPS) activity;
SEQ ID NO: 83 or a sequence 95% identical thereto having glucosylglycerol phosphate phosphatase (GPP) activity;
SEQ ID NO: 85 or a sequence 95% identical thereto having mannosylfructose phosphate synthase (MPS) activity; and
SEQ ID NO: 87 or a sequence 95% identical thereto having mannosylfructose phosphate phosphatase (MPP) activity;
(b) an isolated polynucleotide comprising
SEQ ID NO: 1 or a sequence 95% identical thereto encoding sucrose phosphate synthase/sucrose phosphate phosphatase (ASF) activity;
SEQ ID NO: 3 or a sequence 95% identical thereto encoding sucrose phosphate synthase (SPS) activity;
SEQ ID NO: 5 or a sequence 95% identical thereto encoding sucrose phosphate phosphatase (SPP) activity;
SEQ ID NO: 76 or a sequence 95% identical thereto encoding trehalose phosphate synthase (TPS) activity;
SEQ ID NO: 78 or a sequence 95% identical thereto encoding trehalose phosphate phosphatase (TPP) activity;
SEQ ID NO: 80 or a sequence 95% identical thereto encoding glucosylglycerol phosphate synthase (GPS) activity;
SEQ ID NO: 82 or a sequence 95% identical thereto encoding glucosylglycerol phosphate phosphatase (GPP) activity;
SEQ ID NO: 84 or a sequence 95% identical thereto encoding mannosylfructose phosphate synthase (MPS) activity; and
SEQ ID NO: 86 or a sequence 95% identical thereto encoding mannosylfructose phosphate phosphatase (MPP) activity;
(c) an isolated polynucleotide that hybridizes under stringent conditions to a nucleic acid sequence selected from the group consisting of:
SEQ ID NO: 1, wherein the isolated polynucleotide encodes a polypeptide having ASF activity;
SEQ ID NO: 3, wherein the isolated polynucleotide encodes a polypeptide having SPS activity;
SEQ ID NO: 5, wherein the isolated polynucleotide encodes a polypeptide having SPP activity;
SEQ ID NO: 76, wherein the isolated polynucleotide encodes a polypeptide having TPS activity;
SEQ ID NO: 78, wherein the isolated polynucleotide encodes a polypeptide having TPP activity;
SEQ ID NO: 80, wherein the isolated polynucleotide encodes a polypeptide having GPS activity;
SEQ ID NO: 82, wherein the isolated polynucleotide encodes a polypeptide having GPP activity;
SEQ ID NO: 84, wherein the isolated polynucleotide encodes a polypeptide having MPS activity;
SEQ ID NO: 86, wherein the isolated polynucleotide encodes a polypeptide having MPP activity;
wherein said stringent conditions comprise incubation at 65° C. in a solution comprising 6×SSC (0.9 M sodium chloride and 0.09 M sodium citrate); and
(d) an isolated polynucleotide complementary to the polynucleotide sequence of (a), (b), or (c).

26. The method of claim 25, wherein at least one of the following features is satisfied:
(i) monomers of the accumulated disaccharide are endogenous to the photosynthetic microorganism;
(ii) the photosynthetic microorganism comprises a cyanobacterium cell, a photosynthetic bacteria; or a green algae;
(iii) the photosynthetic microorganism comprises a cyanobacterium cell;
(iv) the photosynthetic microorganism comprises a Synechococcus or a Synechocystis;
(v) the promoter is an inducible promoter;
(vi) the promoter is selected from the group consisting of carB, nirA, psbAII, dnaK, kaiA, and λ_{PR};
(vii) the DNA construct comprises a nucleotide sequence selected from the group consisting of SEQ ID NO: 19 (pLybAL11 encoding asf); SEQ ID NO: 20 (pLybAL12 encoding asf); SEQ ID NO: 44 (pLybAL15 encoding asf); SEQ ID NO: 45 (pLybAL16 encoding asf); SEQ ID NO: 46 (pLybAL17 encoding asf); SEQ ID NO: 47 (pLybAL18 encoding asf); SEQ ID NO: 48 (pLybAL19 encoding asf); SEQ ID NO: 49 (pLybAL21 encoding asf); SEQ ID NO: 50 (pLybAL22 encoding asf); SEQ ID NO: 51 (pLybAL13f encoding asf); SEQ ID NO: 52 (pLyAL13r encoding asf); SEQ ID NO: 53 (pLybAL14f encoding asf); SEQ ID NO: 54 (pLybAL14r encoding asf); SEQ ID NO: 65 (pLybAL7f encoding asf); SEQ ID NO: 69 (pLybAL8f encoding asf); SEQ ID NO: 118 (pLybAL23 encoding tps and tpp); SEQ ID NO: 121 (pLybAL28 encoding tps and tpp); SEQ ID NO: 122 (pLybAL29 encoding tps and tpp); SEQ ID NO: 123 (pLybAL30 encoding tps and tpp); SEQ ID NO: 124 (pLybAL31 encoding tps and tpp); SEQ ID NO: 125 (pLybAL36 encoding tps and tpp); SEQ ID NO: 126 (pLybAL37 encoding tps and tpp); SEQ ID NO: 130 (pLybAL24 encoding tps and tpp); and SEQ ID NO: 133 (pLybAL33 encoding tps and tpp);
(viii) the photosynthetic microorganism accumulates at least about 0.1 micrograms of the disaccharide per minute per gram dry biomass;
(ix) the photosynthetic microorganism accumulates at least about 0.1 micrograms of the disaccharide per minute per gram dry biomass up to about 10 micrograms of the disaccharide per minute per gram dry biomass;
(x) the photosynthetic microorganism does not comprise a nucleotide sequence selected from the group consisting of SEQ ID NO: 70, SEQ ID NO: 72, and SEQ ID NO: 74, or a nucleotide variant thereof having at least 95% identity thereto and invertase activity or sucraseferridoxin activity;
(xi) the photosynthetic microorganism does not express a polypeptide sequence selected from the group consisting of SEQ ID NO: 71, SEQ ID NO: 73, and SEQ ID NO: 75, or a polypeptide variant thereof having at least 95% identity thereto and invertase activity or sucraseferridoxin activity;
(xii) the photosynthetic microorganism expresses a small interfering RNA specific a nucleotide sequence selected from the group consisting of SEQ ID NO: 70, SEQ ID NO: 72, and SEQ ID NO: 74, or a nucleotide variant thereof having at least 95% identity thereto and invertase activity or sucraseferridoxin activity; or
(xiii) the photosynthetic microorganism comprises an isolated polynucleotide comprising SEQ ID NO: 94 or a sequence 95% identical thereto encoding an active porin polypeptide; an isolated polynucleotide encoding a polypeptide comprising SEQ ID NO: 95 or a sequence 95% identical thereto and having porin activity; or an isolated polynucleotide comprising SEQ ID NO: 91 (pLybAL32 encoding a porin); wherein the accumulated disaccacharide is sucrose, the cell expresses porin, and the expressed porin secretes the accumulated sucrose from the cell.
